(19) United States
(12) Patent Application Publication
(10) Pub. No.: US 2003/0061632 A1
(43) Pub. Date: Mar. 27, 2003

(54) Polynucleotides useful for modulating transcription

(76) Inventors: Koen Weterings, Nijmegen (NL); Nestor R. Apuya, Culver City, CA (US); Tatiana Tatarinova, Los Angeles, CA (US); Robert B. Goldberg, Topanga, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 09/997,672

(22) Filed: Nov. 28, 2001

Related U.S. Application Data
(60) Provisional application No. 60/253,672, filed on Nov. 28, 2000.

(51) Int. Cl. A01H 5/00; C12N 15/82; C12N 5/04

(52) U.S. Cl. 800/278; 435/320.1; 435/410

(57) ABSTRACT

The invention provides polynucleotides for expression of genes in suspensor cells in plants and methods for using such polynucleotides.

### CROSS REFERENCE OF RELATED PATENT APPLICSTIONS

The present application claims priority to U.S. Provisional Patent Application Serial No. (USSN) 60/253,672, filed Nov. 28, 2000, which is explicitly incorporated herein by reference in its entirety and for all purposes.

### INTRODUCTION

In most higher plants, the first division of the zygote is asymmetric giving rise to two daughter cells differing in size and developmental fate (Goldberg, R. B., et al., Science, 266:605-614 (1994); embryology of angiopsperms (Johri, B. M., ed., 1984); Kaplan, D. R., et al., Plant Cell, 9:1903-1919 (1997); Laux, T., et al., Plant Cell, 9:898-1000 (1997); embryogenesis in angiosperms: A DEVELOPMENTAL AND EXPERIMENTAL STUDY (Raghavan, V., ed. 1986); West, M. A. L., et al., Plant Cell, 5:1361-1369 (1993)). The small terminal, or apical cell, is cytoplasmically dense and differentiates into the embryo proper containing one or two cotyledons and an axis with shoot and root meristems. By contrast, the large, highly-vacuolate basal cell differentiates into the hypophysis and suspensor. The hypophysis contributes to the formation of the root meristem within the embryo proper (van Den Berg, C., et al., Planta Berlin, 205:483-491 (1998)). The suspensor, on the other hand, is a terminally-differentiated embryonic region that anchors the embryo proper to the surrounding maternal tissue, serves as conduit for nutrients and growth regulators supporting embryo-proper development, and degenerates by the end of embryogenesis (Natesh, S., et al., embryology of angiosperms, (B. M. Johri, ed., 1984) 377-444; Schwartz, B. W., et al., cellular and molecular biology of plant seed development, (B. Vasil, ed. 1997) 53-72,; Walthall, E. D., et al., Cell Differentiation, 18:37-44 (1986); Yeung, E. C., et al., Can. A Bot., 57:120-136 (1979); Yeung, E. C., et al., Plant Cell, 5:1371-1381 (1993)).

The suspensor provides a novel opportunity to use molecular biology in order to understand how the zygote gives rise to daughter cells with distinct developmental fates. It is highly differentiated and contains cells that are direct clonal descendents of the basal cell and, ultimately the basal region of the egg (Goldberg, R. B., et al., Science, 266:605-614 (1994); Schwartz, B. W., et al., cellular and molecular biology of plant seed development, (B. Vasil, ed. 1997) 53-72; Yeung, E. C., et al., Plant Cell, 5:1371-1381 (1993)). Fully developed Arabidopsis and tobacco suspensors, for example, are only three to four cell divisions removed from the basal cell (Mansfield, S. G., et al., Canadian Journal of Botany, 69:461-476 (1991); Soueges, R., Compt. Rend. Acad. Sci. Paris, 170:1125-1127 (1920)). It is possible, therefore, that the mechanisms regulating suspensor-specific gene expression are linked directly to the processes specifying the developmental fate of the basal cell. An understanding how suspensor gene expression is regulated should provide insight into the molecular mechanisms specifying the fate of the basal cell.

Scarlet Runner Bean (Phaseolus coccineus) suspensors are approximately 100 times larger than the suspensors of either Arabidopsis or tobacco (Yeung, E. C., et al., Plant Cell, 5:1371-1381 (1993)). Because of their large size, Scarlet Runner Bean suspensors can be microdissected from embryos during the early stages of embryogenesis (e.g., globular stage) and used for cDNA cloning, transcript profiling, and EST sequencing studies in order to identify and investigate suspensor-specific gene sets.

Control of the expression of genes in suspensor cells in plants is useful in the production of plants with a range of desired traits. For example, control of gene expression in suspensor cells can be used to make seedless fruit or to regulate embryo size or shape. These and other advantages are provided by the present application.

### SUMMARY OF THE INVENTION

The present invention provides expression cassettes comprising a promoter sequence comprising SEQ ID NO:10, SEQ ID NO:11 or SEQ ID NO:12 and a promoter polynucleotide with at least basal promoter activity, which promoter sequence is operably linked to a heterologous polynucleotide, wherein when the expression cassette is inserted into a plant, the heterologous polynucleotide is specifically expressed in a suspensor cell and/or basal region of a plant embryo. In some embodiments, the promoter sequence comprises SEQ ID NO:10. In some embodiments, the promoter sequence comprises SEQ ID NO:11. In some embodiments, the promoter sequence comprises SEQ ID NO:12.

In some embodiments, the promoter is operably linked to the heterologous polynucleotide in an antisense orientation. In some embodiments, the promoter is operably linked to the heterologous polynucleotide in a sense orientation.

The invention also provides vectors comprising the above-described expression cassette. The invention also provides host cells comprising the vector.

The invention also provides transgenic plants comprising the expression cassette described above.

The invention also provides methods of constructing a promoter that specifically induces transcription in a plant suspensor cell and/or basal region of a plant embryo. In some embodiments, the methods comprise (i) providing a promoter polynucleotide capable of at least basal promoter activity in a plant; (ii) inserting a nucleic acid comprising SEQ ID NO:10, SEQ ID NO:11 or SEQ ID NO:12 within or adjoining the promoter polynucleotide, thereby constructing a test promoter; and (iii) assaying the test promoter to determine whether the test promoter specifically initiates transcription in a suspensor cell and/or basal region of a plant embryo. In some embodiments, the nucleic acid comprises SEQ ID NO:10. In some embodiments,the nucleic acid comprises SEQ ID NO:11. In some embodiments, the nucleic acid comprises SEQ ID NO:12.

The invention also provides methods of modulating transcription in a plant suspensor cell and/or basal region of a plant embryo. In some embodiments, the methods comprise introducing into a plant an expression cassette of claim 1. In some embodiments, the nucleic acid comprises SEQ ID NO:10. In some embodiments, the nucleic acid comprises SEQ ID NO:11. In some embodiments, the nucleic acid comprises SEQ ID NO:12. In some embodiments, the promoter is operably linked to the heterologous polynucleotide in an antisense orientation. In some embodiments, the promoter is operably linked to the heterologous polynucleotide in a sense orientation.

The present invention provides polynucleotides comprising a promoter control element, which comprises 1) a nucleotide sequence at least 50% identical to nucleotides 3324 to 3580 of SEQ ID NO:1, or 2) a nucleotide sequence that hybridizes to nucleotides 3324 to 3580 of SEQ ID NO:1 under a condition establishing a Tₘ of 20° C. In some embodiments, the isolated polynucleotides of the invention comprise a polynucleotide comprising 1) a nucleotide sequence at least 50% identical to SEQ ID NO:1, or 2) a nucleotide sequence that hybridizes to SEQ ID NO:1 under a condition establishing a Tₘ of 20° C. In some embodiments, the polynucleotides of the invention comprise nucleotides 3324 to 3580 of SEQ ID NO:1. In some embodiments, the polynucleotides of the invention modulate transcription in a cell. In some embodiments, the polynucleotides of the invention specifically modulate transcription in a plant suspensor cell and/or basal region of a plant embryo.

The present invention also provides expression cassettes comprising a promoter sequence comprising a nucleotide sequence at least 50% identical to nucleotides 3324 to 3580 of SEQ ID NO:1 and a promoter polynucleotide with at least basal promoter activity, which promoter polynucleotide is operably linked to a heterologous polynucleotide, wherein when the expression cassette is inserted into a plant, the heterologous polynucleotide is specifically expressed in a suspensor cell and/or basal region of a plant embryo.

The present invention also provides polynucleotides comprising 1) a nucleotide sequence at least 50% identical to SEQ ID NO:1 or nucleotides 1-3154 or SEQ ID NO:6, or 2) a nucleotide sequence that hybridizes to SEQ ID NO:1 or nucleotides 1-3154 or SEQ ID NO:6 under a condition establishing a Tₘ of 20° C. In some embodiments, the isolated polynucleotides further comprise a G654 or C541 polynucleotide operably linked to the promoter. Examples of such polynucleotides include SEQ ID NO:2 and SEQ ID NO:6. Alternatively, the invention provides for a heterologous polynucleotide operably linked to a promoter. In some embodiments, the polynucleotides of the invention comprise a promoter that modulates transcription in a cell. In some embodiments, the polynucleotides of the invention specifically modulate transcription in a plant suspensor cell and/or basal region of a plant embryo.

The present invention also provides for vectors comprising the above-referenced promoter operably linked to a heterologous polynucleotide. For instance, in some embodiments, the promoter is SEQ ID NO:1 or nucleotides 1 to 3154 of SEQ ID NO:6.

The present invention also provides for a host cell comprising the above-referenced promoters. For instance, in some embodiments, the promoter is SEQ ID NO:1 or nucleotides 1 to 3154 of SEQ ID NO:6. In some embodiments, the host cell comprises a vector comprising the promoters of the invention operably linked to a heterologous nucleic acid.

The invention also provides for plants comprising a promoter comprising 1) a nucleotide sequence at least 50% identical to SEQ ID NO:1 or nucleotides 1-3154 or SEQ ID NO:6, or 2) a nucleotide sequence that hybridizes to SEQ ID NO: 1 or nucleotides 1-3154 or SEQ ID NO:6 under a condition establishing a Tₘ of 20° C., wherein the promoter is operably linked to a heterologous polynucleotide. For instance, in some embodiments, the promoter is SEQ ID NO:1 or nucleotides 1 to 3154 of SEQ ID NO:6. In some embodiments, the plant comprises a vector comprising the promoters of the invention operably linked to a heterologous nucleic acid.

The invention also provides methods of modulating transcription in a suspensor cell comprising introducing into the plant an expression cassette comprising a promoter comprising 1) a nucleotide sequence at least 50% identical to SEQ ID NO:1 or nucleotides 1-3154 or SEQ ID NO:6, or 2) a nucleotide sequence that hybridizes to SEQ ID NO:1 or nucleotides 1-3154 or SEQ ID NO:6 under a condition establishing a Tₘ of 20° C. For instance, in some embodiments, the promoter is SEQ ID NO:1 or nucleotides 1 to 3154 of SEQ ID NO:6. In some embodiments, a G654 or C541 polynucleotide is operably linked to the promoter. In some embodiments, the promoter is operably linked to a heterologous polynucleotide. In some embodiments, the promoter is operably linked to the heterologous polynucleotide in an antisense orientation.

The present invention also provides isolated nucleic acids comprising a polynucleotide sequence, or complement thereof, encoding a G654 polypeptide at least 50% identical to SEQ ID NO:3 or a C541 polypeptide at least 50% identical to SEQ ID NO:7. In some embodiments, the G654 polypeptide is SEQ ID NO:3. In some embodiments, the C541 polypeptide is SEQ ID NO:7. In some embodiments, the polynucleotide is operably linked to a promoter. For example, the promoter can be a constitutive promoter. In some embodiments, the polynucleotide is linked to the promoter in an antisense orientation.

The invention also provides an expression cassette comprising a promoter operably linked to a heterologous polynucleotide, or complement thereof, encoding a G654 or C541 polypeptide at least 50% identical to SEQ ID NO:3 or SEQ ID NO:7, respectively. In some embodiments, the G654 polynucleotide comprises nucleotides 4242 to 4901 of SEQ ID NO:2. In some embodiments, the C541 polynucleotide comprises nucleotides 3155 to 3552 of SEQ ID NO:6. In some embodiments, the polynucleotide is operably linked to a promoter. For example, the promoter can be a constitutive promoter. In some embodiments, the polynucleotide is linked to the promoter in an antisense orientation.

The present invention also provides for host cells and transgenic plants comprising an exogenous nucleic acid comprising a polynucleotide, or complement thereof, encoding a G654 polypeptide at least 50% identical to SEQ ID NO:3 or a C541 polypeptide at least 50% identical to SEQ ID NO:7.

The present invention also provides for isolated polypeptides comprising an amino acid sequence at least 50% identical to SEQ ID NO:3 or SEQ ID NO:7. The invention also provides for antibodies capable of binding the isolated polypeptides.

The invention also provides methods of introducing an isolated polynucleotide into a host cell. The method comprises providing an isolated polynucleotide that comprises 1) a nucleotide sequence at least 50% identical to SEQ ID NO:1 or nucleotides 1-3154 or SEQ ID NO:6, or 2) a nucleotide sequence that hybridizes to SEQ ID NO: 1 or nucleotides 1-3154 or SEQ ID NO:6 under a condition establishing a Tₘ of 20° C. The method also provides contacting the polynucleotide with the host cell under conditions that permit insertion of the polynucleotide into the host cell.

The invention also provides methods of detecting a polynucleotide in a sample. The methods comprise providing a polynucleotide that comprises 1) a nucleotide sequence at least 50% identical to SEQ ID NO: 1 or nucleotides 1-3154 or SEQ ID NO:6, or 2) a nucleotide sequence that hybridizes to SEQ ID NO: 1 or nucleotides 1-3154 or SEQ ID NO:6 under a condition establishing a Tₘ of 20° C. The method also comprises contacting the polynucleotide with a sample under conditions that permit a comparison of the sequence the polynucleotide with a sequence of DNA in the sample and analyzing the result of the comparison. In some embodiments, the polynucleotide and the sample are contacted under conditions that permit formation of a duplex between complementary nucleic acid sequences.

The present invention also provides polynucleotides comprising SEQ ID NO:10 or SEQ ID NO:11. In some embodiments, the polynucleotides of the invention comprise an expression cassette comprising a promoter sequence comprising SEQ ID NO:10 or SEQ ID NO:11 and a promoter polynucleotide with at least basal promoter activity, which promoter polynucleotide is operably linked to a heterologous polynucleotide, wherein when the expression cassette is inserted into a plant, the heterologous polynucleotide is specifically expressed in a suspensor cell and/or basal region of a plant embryo.

The invention also provides methods of constructing a promoter that specifically induces transcription in a plant suspensor cell and/or basal region of a plant embryo, the method comprising (i) providing a promoter polynucleotide capable of at least basal promoter activity in a plant; (ii) inserting a nucleic acid comprising SEQ ID NO:10 or SEQ ID NO:11 within or adjoining the promoter polynucleotide, thereby constructing a test promoter; and (iii) assaying the test promoter to determine whether the test promoter specifically initiates transcription in a suspensor cell and/or basal region of a plant embryo. In some embodiments, the nucleic acid is SEQ ID NO:10 or SEQ ID NO:11.

### DEFINITIONS

The term “basal promoter activity” refers to the ability of a polynucleotide sequence to initiate transcription of an operably linked polynucleotide. Typically, basal activity will provide a low level of constitutive expression that is not inducible under most conditions or that is not cell-specific under most conditions. A basal promoter typically comprises a TATA box and transcriptional start sequence, but does not contain additional stimulatory and repressive elements. An exemplary plant minimal promoter is positions −50 to +8 of the 35S CaMV promoter.

The term “basal region of a plant embryo” refers to the basal cell, i.e., the cell of a two-celled embryo that contacts the suspensor cell. The “basal region” also encompasses derivative or descendent cells of the basal cell.

The term “chimeric” is used to describe polynucleotides or genes, as defined supra, or constructs wherein at least two of the elements of the polynucleotide or gene or construct, such as the promoter and the polynucleotide to be transcribed and/or other regulatory sequences and/or filler sequences and/or complements thereof, are heterologous to each other.

Promoters referred to herein as “constitutive promoters” actively promote transcription under most, but not necessarily all, environmental conditions and states of development or cell differentiation. Examples of constitutive promoters include the cauliflower mosaic virus (CaMV) 35S transcript initiation region and the 1′ or 2′ promoter derived from T-DNA of Agrobacterium tumefaciens, and other transcription initiation regions from various plant genes, such as the maize ubiquitin-1 promoter, known to those of skill.

“Domains” are fingerprints or signatures that can be used to characterize protein families and/or parts of proteins. Such fingerprints or signatures can comprise conserved (1) primary sequence, (2) secondary structure, and/or (3) three-dimensional conformation. A similar analysis can be applied to polynucleotides. Generally, each domain has been associated with either a conserved primary sequence or a sequence motif. Generally these conserved primary sequence motifs have been correlated with specific in vitro and/or in vivo activities. A domain can be any length, including the entirety of the polynucleotide to be transcribed. Examples of domains include, without limitation, AP2, helicase, homeobox, zinc finger, etc.

The term “endogenous,” within the context of the current invention refers to any polynucleotide, polypeptide or protein sequence which is a natural part of a cell or organisms regenerated from said cell.

An “enhancer” is a DNA regulatory element that can increase the steady state level of a transcript, usually by increasing the rate of transcription initiation. Enhancers usually exert their effect regardless of the distance, upstream or downstream location, or orientation of the enhancer relative to the start site of transcription. In contrast, a “suppressor” is a corresponding DNA regulatory element that decreases the steady state level of a transcript, again usually by affecting the rate of transcription initiation. The essential activity of enhancer and suppressor elements is to bind a protein factor(s). Such binding can be assayed, for example, by methods described below. The binding is typically in a manner that influences the steady state level of a transcript in a cell or in an in vitro transcription extract.

As referred to within, “exogenous” is any polynucleotide, polypeptide or protein sequence, whether chimeric or not, that is introduced into the genome of a host cell or organism regenerated from said host cell by any means other than by a sexual cross. Examples of means by which this can be accomplished are described below, and include Agrobacterium-mediated transformation (of dicots—e.g. Salomon et al. EMBO J. 3:141 (1984); Herrera-Estrella et al. EMBO J. 2:987 (1983); of monocots, representative papers are those by Escudero et al, Plant J. 10:355 (1996), Ishida et al., Nature Biotechnology 14:745 (1996), May et al., Bio/Technology 13:486 (1995)), biolistic methods (Armaleo et al., Current Genetics 17:97 1990)), electroporation, in planta techniques, and the like. Such a plant containing the exogenous nucleic acid is referred to here as a T₀ for the primary transgenic plant and T₁ for the first generation. The term “exogenous” as used herein is also intended to encompass inserting a naturally found element into a non-naturally found location.

An “expression cassette” refers to a nucleic acid construct, which when introduced into a host cell, results in transcription and/or translation of an RNA or polypeptide, respectively. Antisense or sense constructs that are not or cannot be translated are expressly included by this definition.

The term “gene,” as used in the context of the current invention, encompasses all regulatory and coding sequence contiguously associated with a single hereditary unit with a genetic function (see FIG. 1). Genes can include non-coding sequences that modulate the genetic function that include, but are not limited to, those that specify polyadenylation, transcriptional regulation, DNA conformation, chromatin conformation, extent and position of base methylation and binding sites of proteins that control all of these. Genes encoding proteins are comprised of “exons” (coding sequences), which may be interrupted by “introns” (non-coding sequences). In some instances complexes of a plurality of protein or nucleic acids or other molecules, or of any two of the above, may be required for a gene's function. On the other hand, a gene's genetic function may require only RNA expression or protein production, or may only require binding of proteins and/or nucleic acids without associated expression. In certain cases, genes adjacent to one another may share sequence in such a way that one gene will overlap the other. A gene can be found within the genome of an organism, in an artificial chromosome, in a plasmid, in any other sort of vector, or as a separate isolated entity.

A “G564 polynucleotide” is a nucleic acid sequence or subsequence that encodes a polypeptide with substantial identity (as defined below) to SEQ ID NO:3 or SEQ ID NO:5. Alternatively, a G564 polynucleotide includes polynucleotide sequences that are substantially identical to SEQ ID NO:1, SEQ ID NO:2, or SEQ ID NO:4 or that hybridize to SEQ ID NO:1, SEQ ID NO:2, or SEQ ID NO:4 under defined conditions.

A “promoter from a G564 gene” or “G564 promoter” will typically be about 500 to about 5000 nucleotides in length, usually from about 2500 to 4000. Exemplary promoter sequences are shown as SEQ ID NO:1 or nucleotides 1-4242 of SEQ ID NO:2. A G564 promoter can also be identified by its ability to direct expression in suspensor cells. “Increased or enhanced G564 activity or expression of the G564 gene” refers to an augmented change in G564 activity. Examples of such increased activity or expression include the following. G564 activity or expression of the G564 gene is increased above the level of that in wild-type, non-transgenic control plants (i.e. the quantity of G564 activity or expression of the G564 gene is increased). G564 activity or expression of the G564 gene is in an organ, tissue or cell where it is not normally detected in wild-type, non-transgenic control plants (i.e. spatial distribution of G564 activity or expression of the G564 gene is increased). G564 activity or expression is increased when G564 activity or expression of the G564 gene is present in an organ, tissue or cell for a longer period than in a wild-type, non- transgenic controls (i.e. duration of G564 activity or expression of the G564 gene is increased).

A “C541 polynucleotide” is a nucleic acid sequence or subsequence that encodes a polypeptide with substantial identity (as defined below) to SEQ ID NO:7 or SEQ ID NO:9. Alternatively, a C541 polynucleotide includes polynucleotide sequences that are substantially identical to SEQ ID NO:6, or SEQ ID NO:8 or that hybridize to SEQ ID NO:6 or SEQ ID NO:8 under defined conditions.

A “promoter from a C541 gene” or “C541 promoter” will typically be about 500 to about 5000 nucleotides in length, usually from about 2500 to 4000. Exemplary promoter sequences are shown as nucleotides 1-3154 of SEQ ID NO:6 or nucleotides 1-1609 of SEQ ID NO:8. A C541 promoter can also be identified by its ability to direct expression in suspensor cells.

“Increased or enhanced C541 activity or expression of the C541 gene” refers to an augmented change in C541 activity. Examples of such increased activity or expression include the following. C541 activity or expression of the C541 gene is increased above the level of that in wild-type, non-transgenic control plants (i.e. the quantity of C541 activity or expression of the C541 gene is increased). C541 activity or expression of the C541 gene is in an organ, tissue or cell where it is not normally detected in wild-type, non-transgenic control plants (i.e. spatial distribution of C541 activity or expression of the C541 gene is increased). C541 activity or expression is increased when C541 activity or expression of the C541 gene is present in an organ, tissue or cell for a longer period than in a wild-type, non-transgenic controls (i.e. duration of C541 activity or expression of the C541 gene is increased).

“Inserting a first polynucleotide within or adjoining” a second polynucleotide is discussed below. “Inserting a first polynucleotide within a second polynucleotide” refers to manipulating or constructing a first and second polynucleotide such that the first polynucleotide interrupts the second polynucleotide (e.g., the first polynucleotide is inserted between the 5′ end and the 3′ end of the second polynucleotide). “Inserting a first polynucleotide adjoining a second polynucleotide” refers to manipulating or constructing a polynucleotide such that the first and second polynucleotides are linked, i.e., the first polynucleotide is adjacent to the second polynucleotide. Of course, one of skill in the art will recognize that the first and the second polynucleotide can be linked in either orientations (e.g., 1→2 or 2→1) or can be linked via a polynucleotide spacer. In the context of promoter sequences, polynucleotides comprising TATA boxes and other basal promoter elements are typically at the 3′ end of a promoter and can be operably linked at their 3′ end to a polynucleotide that is to be transcribed. Moreover, in some embodiments, promoter sequences comprise fewer than 10,000 base pairs, more typically fewer than 5,000 base pairs, sometimes fewer than 3,000, 1,000 or 500 base pairs. However, as noted elsewhere within this application, enhancer elements can function independently of their distance from a basal promoter. Therefore, in some embodiments, the active elements of a promoter can be separated by more than 10,000 base pairs.

“Heterologous sequences” are those that are not operatively linked or are not contiguous to each other in nature. For example, a promoter from corn is considered heterologous to an Arabidopsis coding region sequence. Also, a promoter from a gene encoding a growth factor from maize is considered heterologous to a sequence encoding the maize receptor for the growth factor. Regulatory element sequences, such as UTRs or 3′ end termination sequences that do not originate in nature from the same gene as the coding sequence originates from, are considered heterologous to said coding sequence. Elements operatively linked in nature and contiguous to each other are not heterologous to each other.

In the current invention, a “homologous” gene or polynucleotide or polypeptide refers to a gene or polynucleotide or polypeptide that shares sequence similarity with the gene or polynucleotide or polypeptide of interest. This similarity may be in only a fragment of the sequence and often represents a functional domain such as, examples including without limitation a DNA binding domain or a domain with tyrosine kinase activity. The functional activities of homologous polynucleotide are not necessarily the same.

An “inducible promoter” in the context of the current invention refers to a promoter, the activity of which is influenced by certain conditions, such as light, temperature, chemical concentration, protein concentration, conditions in an organism, cell, or organelle, etc. A typical example of an inducible promoter, which can be utilized with the polynucleotides of the present invention, is PARSK1, the promoter from an Arabidopsis gene encoding a serine-threonine kinase enzyme, and which promoter is induced by dehydration, abscissic acid and sodium chloride (Wang and Goodman, Plant J. 8:37 (1995)). Examples of environmental conditions that may affect transcription by inducible promoters include anaerobic conditions, elevated temperature, the presence or absence of a nutrient or other chemical compound or the presence of light.

As used herein, the phrase “modulate transcription” describes the biological activity of a promoter sequence or promoter control element. Such modulation includes, without limitation, includes up- and down-regulation of initiation of transcription, rate of transcription, and/or transcription levels.

In the current invention, “mutant” refers to a heritable change in nucleotide sequence at a specific location. Mutant genes of the current invention may or may not have an associated identifiable phenotype.

An “operable linkage” is a linkage in which a promoter sequence or promoter control element is connected to a polynucleotide sequence (or sequences) in such a way as to place transcription of the polynucleotide sequence under the influence or control of the promoter or promoter control element. Two DNA sequences (such as a polynucleotide to be transcribed and a promoter sequence linked to the 5′ end of the polynucleotide to be transcribed) are said to be operably linked if induction of promoter finction results in the transcription of mRNA encoding the polynucleotide and if the nature of the linkage between the two DNA sequences does not (1) result in the introduction of a frame-shift mutation, (2) interfere with the ability of the promoter sequence to direct the expression of the protein, antisense RNA or ribozyme, or (3) interfere with the ability of the DNA template to be transcribed. Thus, a promoter sequence would be operably linked to a polynucleotide sequence if the promoter was capable of effecting transcription of that polynucleotide sequence.

“Orthologous” is a term used herein to describe a relationship between two or more polynucleotides or proteins. Two polynucleotides or proteins are “orthologous” to one another if they serve a similar function in different organisms. In general, orthologous polynucleotides or proteins will have similar catalytic finctions (when they encode enzymes) or will serve similar structural finctions (when they encode proteins or RNA that form part of the ultrastructure of a cell).

“Percentage of sequence identity,” as used herein, is determined by comparing two optimally aligned sequences over a comparison window, where the fragment of the polynucleotide or amino acid sequence in the comparison window may comprise additions or deletions (e.g., gaps or overhangs) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sc. (USA) 85: 2444 (1988), by computerized implementations of these algorithms (GAP, BESTFIT, BLAST, PASTA, and TFASTA in the Wisconsin Genetics Software Package, Genetics Computer Group (GCG), 575 Science Dr., Madison, Wis.), or by inspection. Given that two sequences have been identified for comparison, GAP and BESTFIT are preferably employed to determine their optimal alignment. Typically, the default values of 5.00 for gap weight and 0.30 for gap weight length are used.

A “plant promoter” is a promoter capable of initiating transcription in plant cells and can modulate transcription of a polynucleotide. Such promoters need not be of plant origin. For example, promoters derived from plant viruses, such as the CaMV35S promoter or from Agrobacterium tumefaciens such as the T-DNA promoters, can be plant promoters. A typical example of a plant promoter of plant origin is the maize ubiquitin-1 (ubi-1) promoter known to those of skill.

The term “plant tissue” includes differentiated and undifferentiated tissues or plants, including but not limited to roots, stems, shoots, cotyledons, epicotyl, hypocotyl, leaves, pollen, seeds, tumor tissue and various forms of cells and culture such as single cells, protoplast, embryos, basal and apical cells, suspensor cells and callus tissue. The plant tissue may be in plants or in organ, tissue or cell culture.

“Preferential transcription” is defined as transcription that occurs in a particular pattern of cell types or developmental times or in response to specific stimuli or combination thereof. Non-limiting examples of preferential transcription include: high transcript levels of a desired sequence in suspensor cells; detectable transcript levels of a desired sequence in certain cell types during embryogenesis; and low transcript levels of a desired sequence under drought conditions. Such preferential transcription can be determined by measuring initiation, rate, and/or levels of transcription.

A “promoter” is a DNA sequence that directs the transcription of a polynucleotide. Typically a promoter is located in the 5′ region of a polynucleotide to be transcribed, proximal to the transcriptional start site of such polynucleotide. More typically, promoters are defined as the region upstream of the first exon; more typically, as a region upstream of the first of multiple transcription start sites; more typically, as the region downstream of the preceding gene and upstream of the first of multiple transcription start sites; more typically, the region downstream of the polyA signal and upstream of the first of multiple transcription start sites; even more typically, about 3,000 nucleotides upstream of the ATG of the first exon; even more typically, 2,000 nucleotides upstream of the first of multiple transcription start sites. The promoters of the invention comprise at least a core promoter as defined below. Additionally, the promoter may also include at least one control element such as an upstream element. Such elements include UARs and optionally, other DNA sequences that affect transcription of a polynucleotide such as a synthetic upstream element.

The term “promoter control element” as used herein describes elements that influence the activity of the promoter. Promoter control elements include transcriptional regulatory sequence determinants such as, but not limited to, enhancers, scaffold/matrix attachment regions, TATA boxes, transcription start locus control regions, UARs, URRs, other transcription factor binding sites and inverted repeats. Exemplary promoter control elements include, e.g., SEQ ID NO:10 and SEQ ID NO:11.

The term “public sequence,” as used in the context of the instant application, refers to any sequence that has been deposited in a publicly accessible database prior to the filing date of the present application. This term encompasses both amino acid and nucleotide sequences. Such sequences are publicly accessible, for example, on the BLAST databases on the NCBI FTP web site (accessible at ncbi.nlm.gov/blast). The database at the NCBI GTP site utilizes “gi” numbers assigned by NCBI as a unique identifier for each sequence in the databases, thereby providing a non-redundant database for sequence from various databases, including GenBank, EMBL, DBBJ, (DNA Database of Japan) and PDB (Brookhaven Protein Data Bank).

The term “regulatory sequence,” as used in the current invention, refers to any nucleotide sequence that influences transcription or translation initiation and rate, or stability and/or mobility of a transcript or polypeptide product. Regulatory sequences include, but are not limited to, promoters, promoter control elements, protein binding sequences, 5′ and 3′ UTRs, transcriptional start sites, termination sequences, polyadenylation sequences, introns, certain sequences within amino acid coding sequences such as secretory signals, protease cleavage sites, etc.

“Related sequences” refer to either a polypeptide or a nucleotide sequence that exhibits some degree of sequence similarity with a reference sequence.

The term “substantial identity” of polynucleotide sequences means that a polynucleotide comprises a sequence that has at least 25% sequence identity. Alternatively, percent identity can be any integer from 25% to 100%. More preferred embodiments include at least: 25%, 30%, 35%, 40%, 45%, 50%, 55%, 60%, 65%, 70%, 75%, 80%, 85%, 90%, 95%, or 99%. compared to a reference sequence using the programs described herein; preferably BLAST using standard parameters, as described below. For instance, promoter sequences of the invention sequences of the invention include nucleic acid sequences that have substantial identity to SEQ ID NO:1 or other sequences of the invention such as nucleotides 1-4582 of SEQ ID NO:4, nucleotides 1-3154 of SEQ ID NO:6 or nucleotides 1-1609 of SEQ ID NO:8. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 40%. Preferred percent identity of polypeptides can be any integer from 40% to 100%. More preferred embodiments include at least 60%, 65%, 70%, 75%, 80%, 85%, 90%, 95%, or 99%. Most preferred embodiments include 67%, 68%, 69%, 70%, 71%, 72%, 73%, 74% and 75%. Polypeptides which are “substantially similar” share sequences as noted above except that residue positions which are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, aspartic acid-glutamic acid, and asparagine-glutamine.

In the context of the current invention, “specific promoters” refers to a subset of promoters that have a high preference for modulating transcript levels in a specific tissue or organ or cell and/or at a specific time during development of an organism, i.e., that are “specifically initiated” or “specifically modulated” in a specific tissue or at a specific developmental time. By “high preference” is meant at least 3-fold, preferably 5-fold, more preferably at least 10-fold still more preferably at least 20-fold, 50-fold or 100-fold increase in transcript levels under the specific condition and/or a specific tissue over the transcription under any other reference condition and/or in any other reference tissue considered. Examples of tissue-specific promoters under developmental control include promoters that initiate transcription only in certain tissues or organs, such as suspensor cell, root, ovule, fruit, seeds, or flowers. See also “Preferential transcription”.

“Stringency” as used herein is a function of probe length, probe composition (G+C content), and salt concentration, organic solvent concentration, and temperature of hybridization or wash conditions. Stringency is typically compared by the parameter Thd m, which is the temperature at which 50% of the complementary molecules in the hybridization are hybridized, in terms of a temperature differential from Tₘ. High stringency conditions are those providing a condition of Tₘ minus 5° C. to Tₘ minus 10° C. Medium or moderate stringency conditions are those providing Tₘ-minus 20° C. to Tₘ minus 29° C. Low stringency conditions are those providing a condition of Tₘ minus 40° C. to Tₘ minus 48° C. The relationship of hybridization conditions to Tₘ (in °C.) is expressed in the mathematical equation

Tₘ=8.15−16.6(log₁₀[Na⁺])+0.41(%G+C)−(600/N) (1)

where N is the length of the probe. This equation works well for probes 14 to 70 nucleotides in length that are identical to the target sequence. The equation below for Tₘ of DNA-DNA hybrids is useful for probes in the range of 50 to greater than 500 nucleotides, and for conditions that include an organic solvent (formamide).

Tₘ=81.5+16.6log {[Na⁺]/(1+0.7[Na⁺])}+0.41(%G+C)−500/L 0.63(%formamide) (2)

where L is the length of the probe in the hybrid. (P. Tijessen, “Hybridization with Nucleic Acid Probes” in laboratory techniques in biochemistry and molecular biology, (P. C. van der Vliet, ed. 1993)). The Tₘ of equation (2) is affected by the nature of the hybrid; for DNA-RNA hybrids Tₘ is 10-15° C. higher than calculated, for RNA-RNA hybrids Tₘ is 20-25° C. higher. Because the Tₘ decreases about 1° C. for each 1% decrease in homology when a long probe is used (Bonner et al., J. Mol. Biol. 81:123 (1973)), stringency conditions can be adjusted to favor detection of identical genes or related family members.

Equation (2) is derived assuming equilibrium and therefore, hybridizations according to the present invention are most preferably performed under conditions of probe excess and for sufficient time to achieve equilibrium. The time required to reach equilibrium can be shortened by inclusion of a hybridization accelerator such as dextran sulfate or another high volume polymer in the hybridization buffer.

Stringency can be controlled during the hybridization reaction or after hybridization has occurred by altering the salt and temperature conditions of the wash solutions used. The formulas shown above are equally valid when used to compute the stringency of a wash solution. Preferred wash solution stringencies lie within the ranges stated above; high stringency is 5-8° C. below Tₘ, medium or moderate stringency is 26-29° C. below Tₘ and low stringency is 45-48° C. below Tₘ. Hybridization conditions include those in which the salt concentration is less than about 1.0 M sodium ion, typically about 0.1 to 1.0 M sodium ion concentration (or other salts) at pH 7.0 to 8.3 and the temperature is at least about 65° C. or about 60° C., more preferably 55° C. and more preferably 50° C.

A composition containing A is “substantially free” of B when at least 85% by weight of the total A+B in the composition is A. Preferably, A comprises at least about 90% by weight of the total of A+B in the composition, more preferably at least about 95% or even 99% by weight. For example, a plant gene can be substantially free of other plant genes. Other examples include, but are not limited to, ligands substantially free of receptors (and vice versa), a growth factor substantially free of other growth factors and a transcription binding factor substantially free of nucleic acids. the primary TATA motif and the start of transcription.

A “transgenic plant” is a plant having one or more plant cells that contain at least one exogenous polynucleotide introduced by recombinant nucleic acid methods.

In the context of the present invention, a “translational start site” is usually an ATG or AUG in a transcript, often the first ATG or AUG. A single protein encoding transcript, however, may have multiple translational start sites.

“Transcription start site” is used in the current invention to describe the point at which transcription is initiated. This point is typically located about 25 nucleotides downstream from a TFIID binding site, such as a TATA box. Transcription can initiate at one or more sites within the gene, and a single polynucleotide to be transcribed may have multiple transcriptional start sites, some of which may be specific for transcription in a particular cell-type or tissue or organ. “+1” is stated relative to the transcription start site and indicates the first nucleotide in a transcript.

An “Upstream Activating Region” or “UAR” is a position or orientation dependent nucleic acid element that primarily directs tissue, organ, cell type, or environmental regulation of transcript level, usually by affecting the rate of transcription initiation. Corresponding DNA elements that have a transcription inhibitory effect are called herein “Upstream Repressor Regions” or “URR”s. The essential activity of these elements is to bind a protein factor. Such binding can be assayed by methods described below. The binding is typically in a manner that influences the steady state level of a transcript in a cell or in vitro transcription extract.

An “untranslated region” or “UTR” is any contiguous series of nucleotide bases that is transcribed, but is not translated. A 5′ UTR lies between the start site of the transcript and the translation initiation codon and includes the +1 nucleotide. A 3′ UTR lies between the translation termination codon and the end of the transcript. UTRs can have particular functions such as increasing mRNA message stability or translation attenuation. Examples of 3′ UTRs include, but are not limited to polyadenylation signals and transcription termination sequences.

The term “variant” is used herein to denote a polypeptide or protein or polynucleotide molecule that differs from others of its kind in some way. For example, polypeptide and protein variants can consist of changes in amino acid sequence and/or charge and/or post-translational modifications (such as glycosylation, etc). It will be understood that there may be sequence variations within sequence or fragments used or disclosed in this application. Preferably, variants will be such that the sequences have at least 80%, preferably at least 90%, 95, 97, 98, or 99% sequence identity. Variants preferably measure the primary biological finction of the native polypeptide or protein or polynucleotide.

### BRIEF DESCRIPTION OF THE DRAWINGS

FIG. 1 displays a schematic representation of a gene.

FIG. 2 displays the nucleotide sequence of genomic DNA comprising the G564 coding sequence and promoter region from Scarlet Runner Bean (Phaseolus coccineus). The ATG start codon is displayed in bold and underlined nucleotides indicates intron sequences.

FIG. 3 displays the nucleotide sequence of genomic DNA comprising the G564 coding sequence and promoter region from Arabidopsis thaliana. The ATG start codon is displayed in bold and underlined nucleotides indicates intron sequences.

FIG. 4 displays the nucleotide sequence of genomic DNA comprising the C541 coding sequence and promoter region from Scarlet Runner Bean (Phaseolus coccineus). The ATG start codon is displayed in bold and underlined nucleotides indicates intron sequences.

FIG. 5 displays the nucleotide sequence of genomic DNA comprising the C541 coding sequence and promoter region from Arabidopsis thaliana. The ATG start codon is displayed in bold and underlined nucleotides indicates intron sequences.

FIG. 6 is a schematic representation of a deletion analysis of the Scarlet Runner Bean G654 promoter. Suspensor-specific GUS expression was observed in all constructs except the shortest (deleted from the 5′ end to position -662). This figure demonstrates that a suspensor-specific cis-acting sequence is located between positions -921 and -662 (corresponding to nucleotides 3324-3580 of SEQ ID NO:2).

FIG. 7 is a schematic representation of a series of promoter fragments from the Scarlet Runner Bean G564 promoter region fused to a minimal 35S promoter and GUS gene.

FIG. 8 identifies a number of promoter control elements found within sequences -921 to -662 of FIG. 1.

FIG. 9 identifies an additional number of promoter control elements found within the promoter sequences of SEQ ID NOs: 1-4. The column of numbers to the left of the sequences refers to the origin of the sequence. 0 indicates the sequence is from SEQ ID NO:4, 1 is from SEQ ID NO:6, 2 is from SEQ ID NO: 1, and 3 is from SEQ ID NO:8. The first two columns of numbers to the right of the sequences indicate the position of the sequence where “1” is the 5′ most nucleic acid in the genomic clone. The two columns of numbers farthest to the right from the sequences indicate the position of the sequence where the “A” of the ATG is “1”.

### DETAILED DESCRIPTION OF THE INVENTION

A. INTRODUCTION

The present invention provides the identification of two Scarlet Runner Bean mRNAs, designated as C541 and G564, that accumulate specifically within the suspensor of globular-stage embryos. At the pre-globular, or four-cell stage, both C541 and G564 mRNAs are present in the two basal cells, but are absent from the two embryo-proper cells. Expression analysis of a chimeric G564/GUS gene in transgenic tobacco embryos showed that the G564 promoter is active specifically within the suspensor during early embryo development.

The present invention provides polynucleotides comprising promoters and promoter control elements which are capable of modulating transcription.

Such promoters and promoter control elements can be used in combination with native or heterologous promoter fragments, control elements or other regulatory sequences to modulate transcription and/or translation.

Specifically, promoters and control elements of the invention can be used to modulate transcription of a desired polynucleotide, which includes without limitation:

(a) antisense;

(b) ribozymes;

(c) coding sequences; or

(d) fragments thereof.

The promoter also can modulate transcription in a host genome in cis- or in trans-.

In an organism, such as a plant, the promoters and promoter control elements of the instant invention are useful to produce preferential transcription which results in a desired pattern of transcript levels in a particular cells, tissues, or organs, or under particular conditions.

The present invention also provides new suspensor-specific genes useful in genetically engineering plants. Suspensor-specific promoter sequences from the genes of the invention can be used, for instance, to ablate embryos to make seedless fruit, e.g., by expressing gene products toxic to the suspensor and/or surrounding cells such as the embryo itself. The suspensor-specific promoters can also be operably linked to growth regulator genes, such as gene products regulating gibberellin production, thereby modulating embryo size, shape and/or rate of development.

B. Identifying and Isolating Promoter Sequences or Structural Polynucleotides of the Invention

The exemplary promoters and promoter control elements of the present invention (e.g., SEQ ID NO:1 and fragments thereof) were identified from Scarlet Runner bean (Phaseolus coccineus). Additional promoter sequences can be identified as described below. SEQ ID NO:1 and SEQ ID NO:2 includes a promoter region of approximately 4200 base pairs upstream of the ATG start codon.

In addition, the coding sequence of a suspensor-specific gene, designated G564, was identified (e.g., nucleotides 4242 to 4349 and 4513 to 4901 of SEQ ID NO:2). The genus of G564 nucleic acid sequences of the invention includes genes and gene products identified and characterized by analysis using the sequences nucleic acid sequences, nucleotides 4242 to 4349 and 4513 to 4901 of SEQ ID NO:2, as well as nucleotides 4242 to 6986 of SEQ ID NO:2, and protein sequences, including SEQ ID NO:3. G564 sequences of the invention include polypeptide sequences having substantial identify to SEQ ID NO:3. The orthologous Arabidopsis G564 polynucleotide was also identified (SEQ ID NO:4).

In addition, a polynucleotide designated C541 was also isolated from Scarlet Runner Bean (SEQ ID NO:6). The orthologous Arabidopsis C541 sequence is displayed as SEQ ID NO:8. The respective amino acid sequences encoded by the bean and Arabidopsis polynucleotides are SEQ ID NO:7 and SEQ ID NO:9.

The promoter sequences of the invention are useful to modulate transcription of polynucleotides. For example, promoter sequences can be operably linked to a polynucleotide of interest to modulate expression of that polynucleotide in desired tissues. Desired tissues for polynucleotide expression include, e.g, suspensor cells and/or the basal region of a plant embryo, the embryo root meristem as well as the plant root tip and plant root meristem.

Alternatively, promoter sequences of the invention, e.g., SEQ ID NO:1, are useful to modulate expression of polynucleotides in desired plant tissues. In addition, the promoter sequences of the invention can also be introduced into a cell in multiple copies, thereby competing with endogenous promoter sequences for transcription factors. By removing some or all of the transcription factors available for a particular promoter, transcription from those endogenous promoters is modulated.

(1) Cloning Methods

Isolation from genomic libraries of polynucleotides comprising the sequences of the genes, promoters and promoter control elements described in SEQ ID NO: 1 and SEQ ID NO:2 or other polynucleotides of the present invention is possible using known techniques.

For example, polymerase chain reaction (PCR) can amplify the desired polynucleotides utilizing primers designed from sequences in SEQ ID NO: 1, SEQ ID NO:2, SEQ ID NO:4, SEQ ID NO:6 or SEQ ID NO:8. Polynucleotide libraries comprising genomic sequences can be constructed according to Sambrook et al., molecular cloning: a laboratory manual, 2^{nd} Ed. (1989), for example.

Other procedures for isolating polynucleotides comprising the polynucleotide sequences of the invention include, without limitation, tail-PCR, and 5′ rapid amplification of cDNA ends (RACE). For tail-PCR, see, e.g., Liu et al., Plant J 8(3): 457-463 (1995); Liu et al., Genomics 25: 674-681 (1995); Liu et al., Nucl. Acids Res. 21(14): 3333-3334 (1993); and Zoe et al., BioTechniques 27(2): 240-248 (1999);for RACE, see, e.g., PCR Protocols: A Guide to Methods and Applications, (1990) Academic Press, Inc.

(2) Chemical Synthesis

In addition, the genes, promoters and promoter control elements of the invention can be chemically synthesized according to techniques in common use. See, e.g., Beaucage et al., Tet. Lett. 22: 1859 (1981) and U.S. Pat. No. 4,668,777.

Such chemical oligonucleotide synthesis can be carried out using commercially available devices, such as, Biosearch 4600 or 8600 DNA synthesizer, by Applied Biosystems, a division of Perkin-Elmer Corp., Foster City, Calif., USA; and Expedite by Perceptive Biosystems, Framingham, Mass., USA.

Synthetic RNA, including natural and/or analog building blocks, can be synthesized on the Biosearch 8600 machines, see above.

Oligonucleotides can be synthesized and then ligated together to construct the desired polynucleotide.

C. Isolating Related Polvnucleotide Sequences

Included in the present invention are genes, promoters and promoter control elements which are related to those described in SEQ ID NO:1, SEQ ID NO:2, SEQ ID NO:4, SEQ ID NO:6 or SEQ ID NO:8. Such related sequence can be isolated utilizing

nucleotide sequence identity;

coding sequence identity; or

common function or gene products.

Relatives can include both naturally occurring genes and promoters and non-natural gene and promoter sequences. Non-natural related gene or promoters include nucleotide substitutions, insertions or deletions of naturally-occurring gene or promoter sequences that do not substantially affect activity of the polynucleotides (e.g., activity of coding sequences or transcription modulation). For example, the binding of relevant DNA binding proteins can still occur with the non-natural promoter sequences and promoter control elements of the present invention.

According to current knowledge, promoter sequences and promoter control elements exist as functionally important regions, such as protein binding sites, and spacer regions. These spacer regions are apparently required for proper positioning of the protein binding sites. Thus, nucleotide substitutions, insertions and deletions can be tolerated in these spacer regions to a certain degree without loss of function.

In contrast, less variation is permissible in the functionally important regions, since changes in the sequence can interfere with protein binding. Nonetheless, some variation in the functionally important regions is permissible so long as function is conserved. In some embodiments, functionally important regions can include nucleotides 3324 to 3580 of SEQ ID NO:1. As described below, nucleotides 3324 to 3580 of SEQ ID NO:2 are useful for modulating transcriptional activity in suspensor cells and/or basal regions of plant embryos.

The effects of substitutions, insertions and deletions to the promoter sequences or promoter control elements may be to increase or decrease the binding of relevant DNA binding proteins to modulate transcript levels of a polynucleotide to be transcribed. Effects may include tissue-specific or condition-specific modulation of transcript levels of the polypeptide to be transcribed. Polynucleotides representing changes to the nucleotide sequence of the DNA-protein contact region by insertion of additional nucleotides, changes to identity of relevant nucleotides, including use of chemically-modified bases, or deletion of one or more nucleotides are considered encompassed by the present invention.

(1) Relatives Based on Nucleotide Sequence Identity

Included in the present invention are polynucleotides comprising genes or promoters exhibiting nucleotide sequence identity to SEQ ID NO: 1, SEQ ID NO:2, SEQ ID NO:4, SEQ ID NO:6 or SEQ ID NO:8.

Definition

Typically, such related genes or promoters exhibit at least 50%, sometimes at least 60% or at least 70% or at least 80% sequence identity, preferably at least 85%, more preferably at least 90%, and most preferably at least 95%, even more preferably, at least 96%, 97%, 98% or 99% sequence identity compared to SEQ ID NO:1, SEQ ID NO:2, SEQ ID NO:4, SEQ ID NO:6 or SEQ ID NO:8. Indeed, any percent identity represented by an integer between 50-99 is contemplated for the invention. Such sequence identity can be calculated by the algorithms and computers programs described above.

Usually, such sequence identity is exhibited in an alignment region that is at least 75%, usually at least 80%; more usually, at least 85%, more usually at least 90%, and most usually at least 95%, even more usually, at least 96%, 97%, 98% or 99% of the length of a sequence shown in SEQ ID NO: 1.

The percentage of the alignment length is calculated by counting the number of residues of the sequence in region of strongest alignment, e.g., a continuous region of the sequence that contains the greatest number of residues that are identical to the residues between two sequences that are being aligned. The number of residues in the region of strongest alignment is divided by the total residue length of a sequence in SEQ ID NO:1.

These related promoters may exhibit similar preferential transcription as SEQ ID NO:1 or other sequences of the invention such as nucleotides 1-4582 of SEQ ID NO:4, nucleotides 1-3154 of SEQ ID NO:6 or nucleotides 1-1609 of SEQ ID NO:8.

Construction of Polynucleotides

Naturally occurring promoters that exhibit nucleotide sequence identity to those shown in SEQ ID NO:1, SEQ ID NO:2, SEQ ID NO:4, SEQ ID NO:6 or SEQ ID NO:8 can be isolated using the techniques as described above. More specifically, such related promoters can be identified by varying stringencies, as defined above, in typical hybridization procedures such as, Southems or probing of polynucleotide libraries, for example.

Non-natural promoter variants of those shown in SEQ ID NO:1, SEQ ID NO:2, SEQ ID NO:4, SEQ ID NO:6 or SEQ ID NO:8 can be constructed using cloning methods that incorporate the desired nucleotide variation. See, for example, Ho, S. N., et al. Gene 77:51-59 (1989), describing a procedure site directed mutagenesis using PCR.

Any related promoter showing sequence identity to those shown in SEQ ID NO:1, SEQ ID NO:2, SEQ ID NO:4, SEQ ID NO:6 or SEQ ID NO:8 can be chemically synthesized as described above.

Also, the present invention includes non-natural promoters that exhibit the above-sequence identity to those in SEQ ID NO: 1, SEQ ID NO:2, SEQ ID NO:4, SEQ ID NO:6 or SEQ ID NO:8.

The promoters and promoter control elements of the present invention may also be synthesized with 5′ or 3′ extensions, to facilitate additional manipulation, for instance.

(2) Relatives Based on Coding Sequence Identitv

In addition, the present invention includes promoters of genes that comprise exons that encode polypeptide sequences that show sequence identity to the amino acid sequence displayed in SEQ ID NO:3, SEQ ID NO:5, SEQ ID NO:7, or SEQ ID NO:9.

Definition

Typically, the amino acid sequence of the genes comprising these related polynucleotides exhibit at least that exhibit at least 50%, at least 60%, at least 70% or at least 80% sequence identity to SEQ ID NO:3, SEQ ID NO:5, SEQ ID NO:7, or SEQ ID NO:9, preferably at least 85%, more preferably at least 90%, and most preferably at least 95%, even more preferably, at least 96%, 97%, 98% or 99% sequence identity to SEQ ID NO:3, SEQ ID NO:5, SEQ ID NO:7, or SEQ ID NO:9. Such sequence identity can be calculated by the algorithms and computers programs described above.

Usually, such sequence identity is exhibited in an alignment region that is at least 75% of the length of a sequence encoded by SEQ ID NO:2, SEQ ID NO:4, SEQ ID NO:6 or SEQ ID NO:8 or corresponding full-length sequence; more usually at least 80%; more usually, at least 85%, more usually at least 90%, and most usually at least 95%, even more usually, at least 96%, 97%, 98% or 99% of the length of a sequence encoded by SEQ ID NO:2, SEQ ID NO:4, SEQ ID NO:6 or SEQ ID NO:8.

Construction of Polynucleotides

The isolation of sequences from the genes of the invention may be accomplished by a number of techniques. For instance, oligonucleotide probes based on the sequences disclosed here can be used to identify the desired gene in a cDNA or genomic DNA library from a desired plant species. To construct genomic libraries, large segments of genomic DNA are generated by random fragmentation, e.g. using restriction endonucleases, and are ligated with vector DNA to form concatemers that can be packaged into the appropriate vector. To prepare a library of embryo-specific cDNAs, mRNA is isolated from embryos and a cDNA library that contains the gene transcripts is prepared from the mRNA.

The cDNA or genomic library can then be screened using a probe based upon the sequence of a cloned embryo-specific gene such as the polynucleotides disclosed here. Probes may be used to hybridize with genomic DNA or cDNA sequences to isolate homologous genes in the same or different plant species.

Alternatively, the nucleic acids of interest can be amplified from nucleic acid samples using amplification techniques. For instance, polymerase chain reaction (PCR) technology to amplify the sequences of the genes directly from mRNA, from cDNA, from genomic libraries or cDNA libraries. PCR and other in vitro amplification methods may also be useful, for example, to clone nucleic acid sequences that code for proteins to be expressed, to make nucleic acids to use as probes for detecting the presence of the desired mRNA in samples, for nucleic acid sequencing, or for other purposes. Appropriate primers and probes for identifying embryo-specific genes from plant tissues are generated from comparisons of the sequences provided herein. For a general overview of PCR see PCR Protocols: A Guide to Methods and Applications. (Innis, M, Gelfand, D., Sninsky, J. and White, T., eds.), Academic Press, San Diego (1990).

Polynucleotides may also be synthesized by well-known techniques as described in the technical literature. See, e.g., Carruthers et al., Cold Spring Harbor Symp. Quant. Biol. 47:411-418 (1982), and Adams et al., J. Am. Chem. Soc. 105:661(1983). Double stranded DNA fragments may then be obtained either by synthesizing the complementary strand and annealing the strands together under appropriate conditions, or by adding the complementary strand using DNA polymerase with an appropriate primer sequence.

Identified cDNA sequences can be aligned to the genomic sequences to identify the promoter region and sequences, which are located upstream of the 5′UTR and downstream of the preceding gene.

cDNA Isolation

The cDNAs can be isolated by various cloning methods described above. For example, probes and/or primer can be designed utilizing the sequences in SEQ ID NO:2, SEQ ID NO:4, SEQ ID NO:6 or SEQ ID NO:8. See, e.g., Ausubel et al. (1992); and Sambrook et al. (1989).

Such probes and primers can be used to identify cDNAs with a comprising at least one transcription start site. Full-length cDNA libraries are useful to identify cDNAs with at least one transcription start site. Such libraries can be constructed as described in the above-captioned applications in the Related Applications Section. Alternatively, tail-PCR or RACE can be used to isolated the 5′ end of a cDNA.

Genomic Polynucleotide Isolation

Genomic sequences can be isolated with the sequence from the cDNA also found in the 5′ UTR, exons or 3′ UTR for probes and/or primers.

Alternatively, the promoter sequences upstream of the transcription start site or translation start site can be isolated using single primers designed having the portions of cDNA sequences 3′ of the start codon of a sequence (e.g., SEQ ID NO:2, SEQ ID NO:4, SEQ ID NO:6 or SEQ ID NO:8) and used with random primers to isolate the corresponding upstream portion of genomic DNA.

Alternatively the promoters and promoter control elements of the invention can be identified by “walking” upstream from 5′-most portions of cDNA sequences in a genomic DNA library.

The promoter sequences will those 5′ of the transcription start site which can be located using the 5′ end of the corresponding cDNA. Alternatively, the start sites of a transcript can be assessed using primer extension assays (King et al., Gene 242:125 (2000)).

In addition, the 5′ end of the promoter can be identified by either locating the upstream polyA signal or by identifying the cDNA corresponding to the preceding gene using the techniques described above.

D. Identifying Control Elements

(1) Types of Transcription Control Elements

Promoter sequences comprise a number of promoter control elements that are capable of initiating transcription, regulating transcription rates and levels, etc. Promoter control elements modulate transcription when such control elements exhibit their transcription related activities, such as hybridizing to target polynucleotides; binding to repressor proteins, transcription factors, proteins or components of the nuclear matrix; able to act as a methylation site, etc. Promoter control elements include cis acting elements such as

enhancers,

scaffold/matrix attachment regions (S/MARs),

locus control regions (LCRs).

Other promoter control elements include, without limitation:

core or basal promoters,

TATA boxes,

initiator sites,

transcription factor binding sites,

repressor binding sites;

and inverted repeats.

See, e.g., T. Boulikas, J. Cell Biochem., 60, 297-316 (1996).

Promoter Control Elements of the Invention

The promoter control elements of the present invention include those that comprise SEQ ID NO: 1, nucleotides 1-4582 of SEQ ID NO:4, nucleotides 1-3154 of SEQ ID NO:6 or nucleotides 1-1609 of SEQ ID NO:8, and fragments thereof. A particularly preferred fragment comprises nucleotides 3329 to 3475 of SEQ ID NO: 1. As discussed below, this fragment confers suspensor-specific activity to a promoter. Additional promoter control elements include SEQ ID NO:10 and SEQ ID NO:11. Control elements of the invention alone, or as part of a heterologous promoter, are useful for modulation of transcription.

The size of the fragments of SEQ ID NO:1, nucleotides 1-4582 of SEQ ID NO:4, nucleotides 1-3154 of SEQ ID NO:6 or nucleotides 1-1609 of SEQ ID NO:8 can range from 5 bases to about 5 kilobases (kb). Typically, the fragment size is no smaller than 8 bases; more typically, no smaller than 10 or 12; more typically, no smaller than 15 bases; more typically, no smaller than 20 bases; more typically, no smaller than 25 bases; even more typically, no more than 30, 35, 40 or 50 bases.

Usually, the fragment size in no larger than 2 kb bases; more usually, no larger than 1 kb; more usually, no larger than 800 bases; more usually, no larger than 500 bases; even more usually, no more than 250, 200, 150 or 100 bases.

Relatives Based on Nucleotide Sequence Identity

Included in the present invention are promoter control elements exhibiting nucleotide sequence identity to those in SEQ ID NO:1, nucleotides 1-4582 of SEQ ID NO:4, nucleotides 1-3154 of SEQ ID NO:6 or nucleotides 1-1609 of SEQ ID NO:8.

Typically, such related promoters exhibit at least 80% sequence identity, preferably at least 85%, more preferably at least 90%, and most preferably at least 95%, even more preferably, at least 96%, 97%, 98% or 99% sequence identity compared to those shown in SEQ ID NO:1, nucleotides 1-4582 of SEQ ID NO:4, nucleotides 1-3154 of SEQ ID NO:6 or nucleotides 1-1609 of SEQ ID NO:8. Such sequence identity can be calculated by the algorithms and computers programs described above.

Relatives Based on Coding Sequence Identity

In addition, the present invention includes promoter control elements of genes that comprise exons that encode polypeptide sequences that show sequence identity to SEQ ID NO:3, SEQ ID NO:5, SEQ ID NO:7 or SEQ ID NO:9.

Typically, the amino acid sequence of the genes comprising these related promoters exhibit at least 80% sequence identity to those shown in SEQ ID NO:3, SEQ ID NO:5, SEQ ID NO:7 or SEQ ID NO:9, preferably at least 85%, more preferably at least 90%, and most preferably at least 95%, even more preferably, at least 96%, 97%, 98% or 99% sequence identity to SEQ ID NO:3, SEQ ID NO:5, SEQ ID NO:7 or SEQ ID NO:9. Such sequence identity can be calculated by the algorithms and computers programs described above.

Usually, such sequence identity is exhibited in an alignment region that is at least 75% of the length of SEQ ID NO:3, SEQ ID NO:5, SEQ ID NO:7 or SEQ ID NO:9; more usually at least 80%; more usually, at least 85%, more usually at least 90%, and most usually at least 95%, even more usually, at least 96%, 97%, 98% or 99% of the length of SEQ ID NO:3, SEQ ID NO:5, SEQ ID NO:7 or SEQ ID NO:9.

Promoter Control Element Configuration

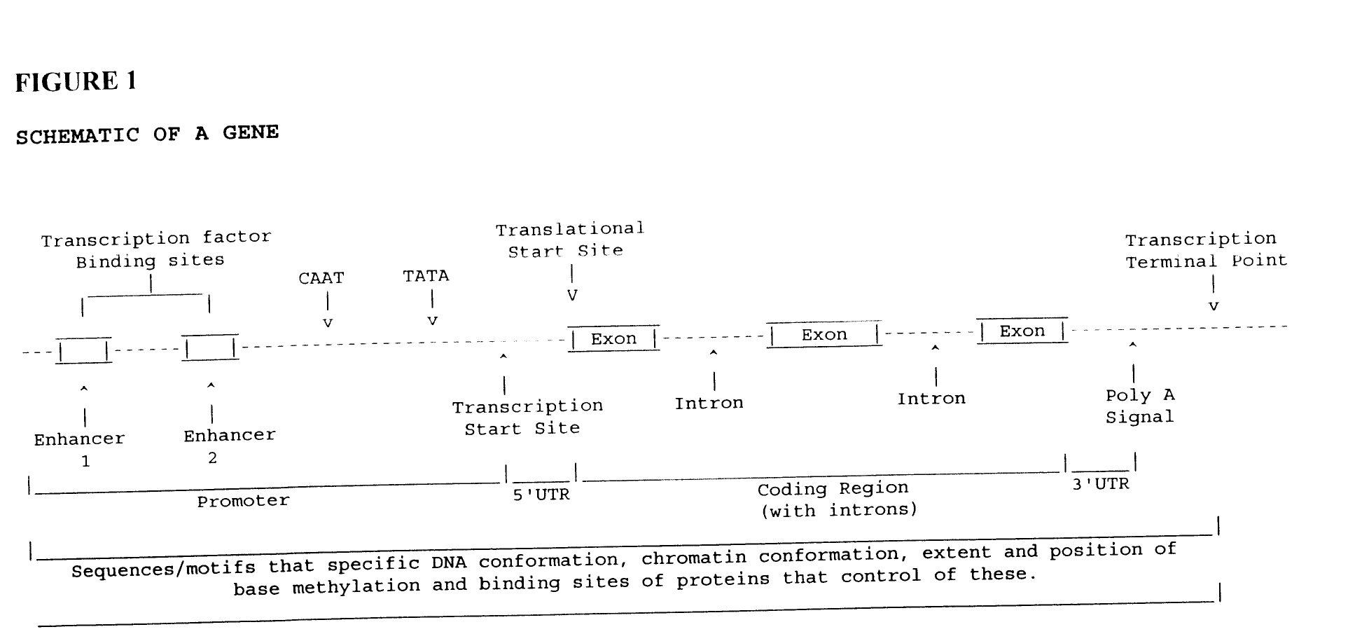

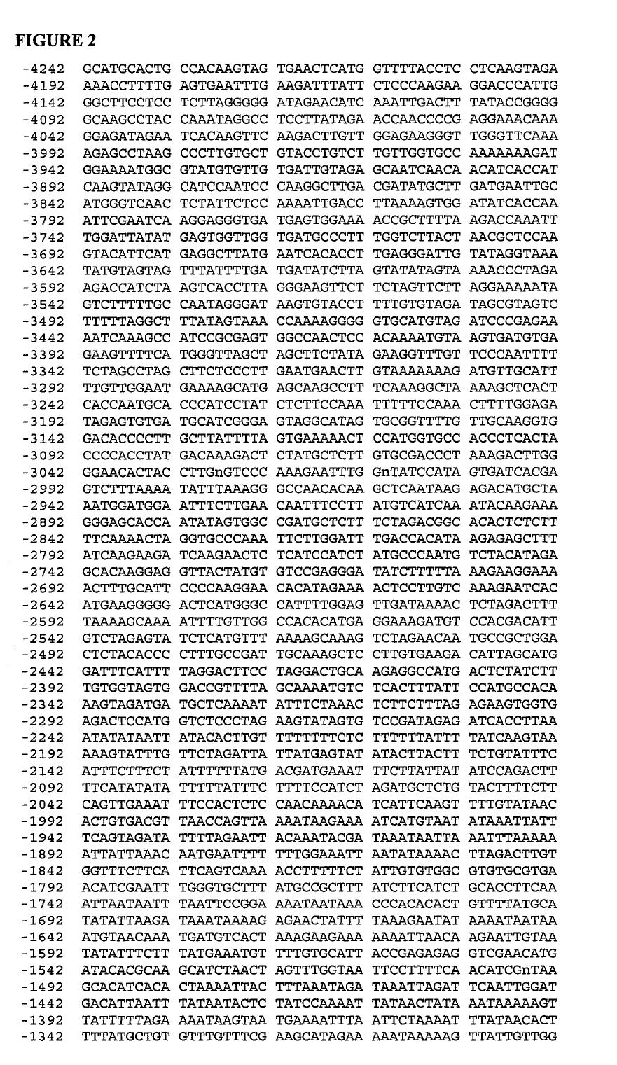

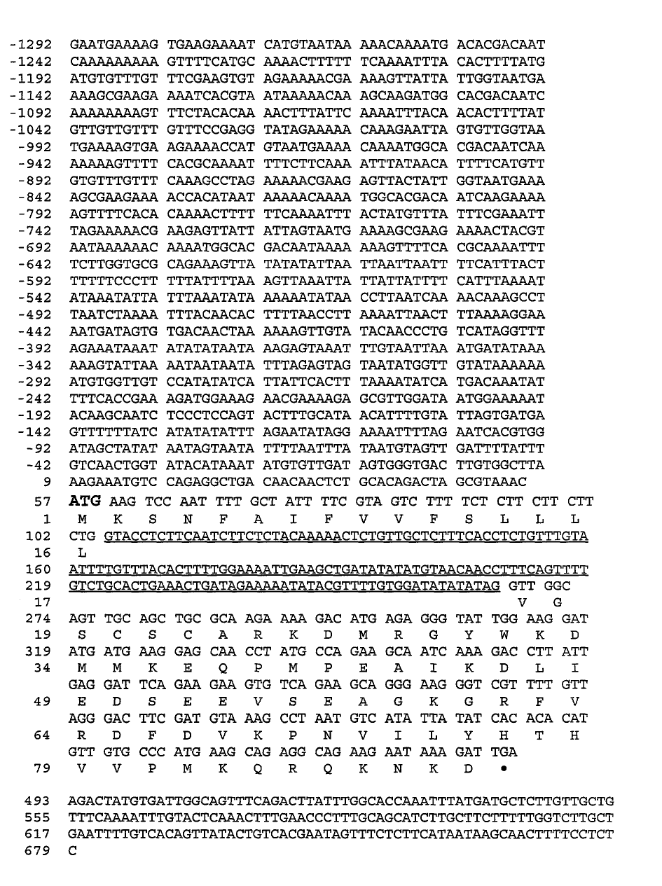

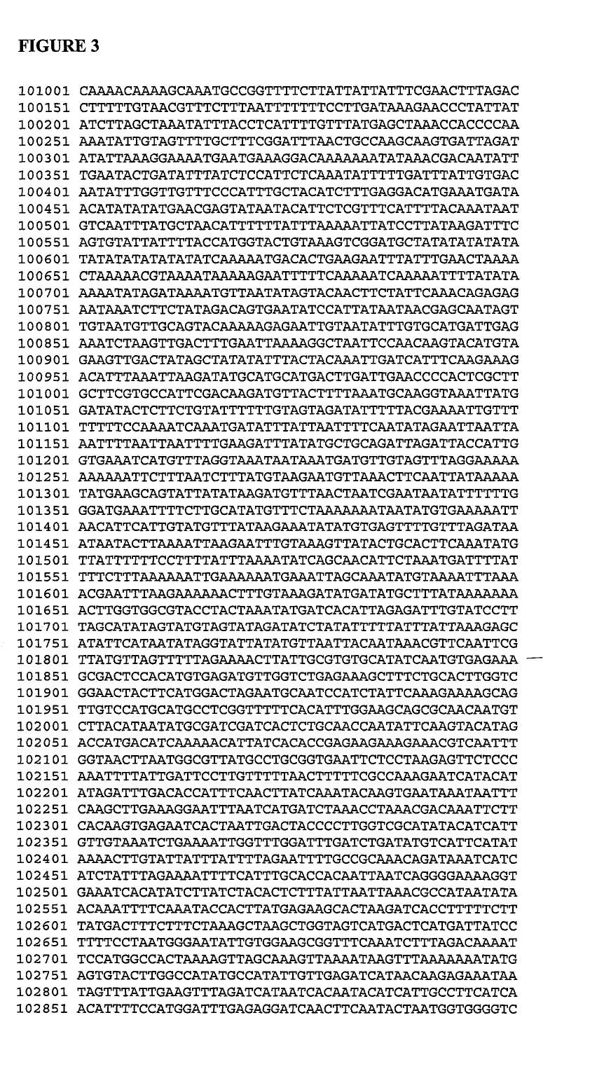

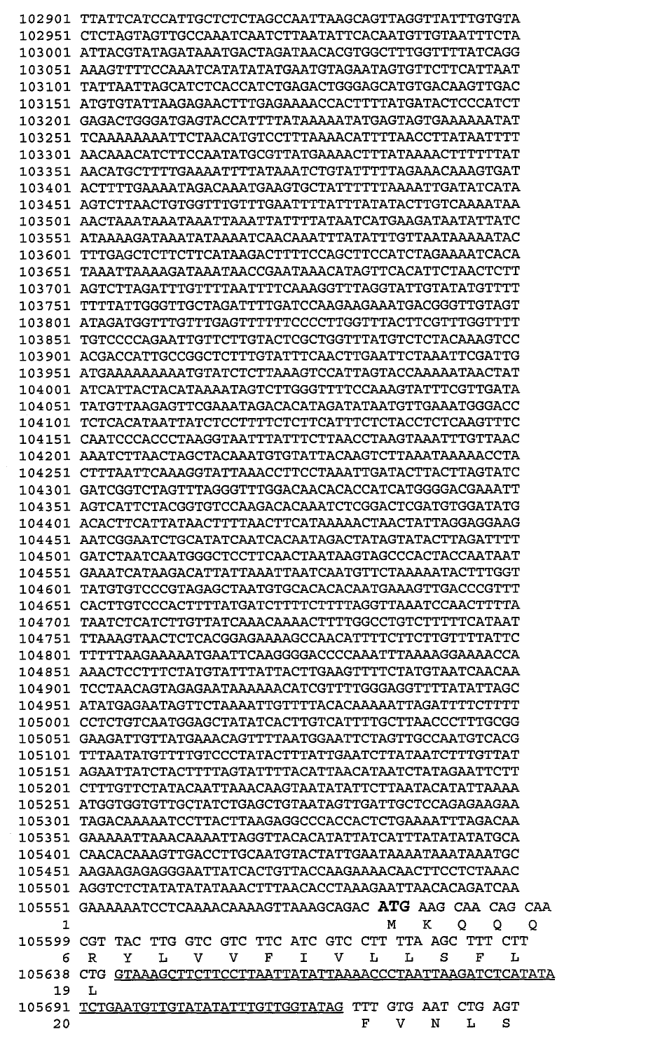

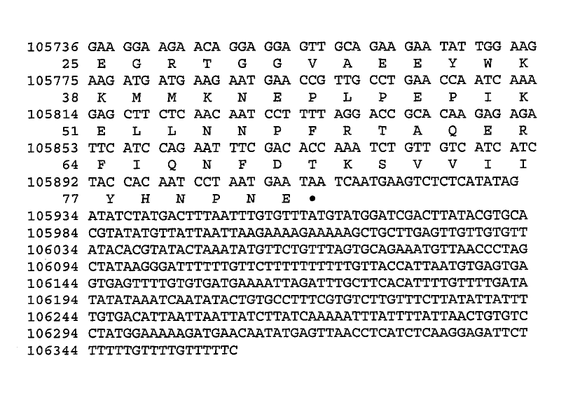

A common configuration of the promoter control elements in RNA polymerase II promoters is shown in FIG. 1.

For more description, see, e.g., T. Werner, Mammalian Genome, 10, 168-175 (1999).

Promoters are generally modular in nature. Promoters can consist of a basal promoter that functions as a site for assembly of a transcription complex comprising an RNA polymerase, for example RNA polymerase II. A typical transcription complex will include additional factors such as TF_{II}B, TF_{II}D, and TF_{II}E. Of these, TF_{II}D appears to be the only one to bind DNA directly. The promoter might also contain one or more promoter control elements such as the elements discussed above. These additional control elements may function as binding sites for additional transcription factors that have the function of modulating the level of transcription with respect to tissue specificity and of transcriptional responses to particular environmental or nutritional factors, and the like.

One type of promoter control elements are polynucleotide sequences representing binding sites for proteins. Typically, within a particular functional module, protein binding sites constitute regions of 5 to 60, preferably 10 to 30, more preferably 10 to 20 nucleotides. Within such binding sites, there are typically 2 to 6 nucleotides that specifically contact amino acids of the nucleic acid binding protein.

The protein binding sites are usually separated from each other by 10 to several hundred nucleotides, typically by 15 to 150 nucleotides, often by 20 to 50 nucleotides.

Further, protein binding sites in promoter control elements often display dyad symmetry in their sequence. Such elements can bind several different proteins, and/or a plurality of sites can bind the same protein. Both types of elements may be combined in a region of 50 to 1,000 base pairs.

Binding sites for any specific factor have been known to occur almost anywhere in a promoter. For example, functional AP-1 binding sites can be located far upstream, as in the rat bone sialoprotein gene, where an AP-1 site located about 900 nucleotides upstream of the transcription start site suppresses expression. Yamauchi et al., Matrix Biol., 15, 119-130 (1996). Alternatively, an AP-1 site located close to the transcription start site plays an important role in the expression of Moloney murine leukemia virus. Sap et al., Nature, 340, 242-244 (1989).

(2) Those Identifiable by Bioinformatics

Promoter control elements from the promoters of the instant invention can be identified utilizing bioinformatic or computer driven techniques.

One method uses a computer program AlignACE to identify regulatory motifs in genes that exhibit common preferential transcription across a number of time points. The program identifies common sequence motifs in such genes. See, Roth et al., Nature Biotechnol. 16: 949-945 (1998); Tavazoie et al., Nat Genet 22(3):281-5 (1999).

Genomatix, also makes available a GEMS Launcher program and other programs to identify promoter control elements and configuration of such elements. Genomatix is located in Munich, Germany.

Other references also describe detection of promoter modules by models independent of overall nucleotide sequence similarity. See, e.g., Klingenhoff et al., Bioinformatics 15, 180-186 (1999).

Protein binding sites of promoters can be identified as reported in Frech, et al., Nucleic Acids Research, Vol. 21, No. 7, 1655-1664 (1993).

Other programs used to identify protein binding sites include, for example, Signal Scan, Prestridge et al., Comput. Appl. Biosci. 12: 157-160 (1996); Matrix Search, Chen et al., Comput. Appl. Biosci. 11: 563-566 (1995), available as part of Signal Scan 4.0; MatInspector, Ghosh et al., Nucl. Acid Res. 21: 3117-3118 (1993) available http://ww.gsf.de/cgi-bin/matsearch.pl; ConsInspector, Frech et al., Nucl. Acids Res. 21: 1655-1664 (1993), available at ftp://ariane.gsf.de/pub/dos; TFSearch; and TESS.

Frech et al., “Software for the analysis of DNA sequence elements of transcription” in bioinformatics & sequence analysis, Vol. 13, no. 1, 89-97 (1997) is a review of different software for analysis of promoter control elements. This paper also reports the usefulness of matrix-based approaches to yield more specific results.

For other procedures, see, Fickett et al., Curr. Op. Biotechnol. 11: 19-24 (2000); and Quandt et al., Nucleic Acids Res. 23, 4878-4884 (1995).

(3) Those Identifiable by In-Vitro and In-Vivo Assays

Promoter control elements also can be identified with in-vitro assays, such as transcription detection methods; and with in-vivo assays, such as enhancer trapping protocols.

In-Vitro Assavs

Examples of in vitro assays include detection of binding of protein factors that bind promoter control elements. Fragments of the instant promoters can be used to identify the location of promoter control elements. Another option for obtaining a promoter control element with desired properties is to modify known promoter sequences. This is based on the fact that the function of a promoter is dependent on the interplay of regulatory proteins that bind to specific, discrete nucleotide sequences in the promoter, termed motifs. Such interplay subsequently affects the general transcription machinery and regulates transcription efficiency. These proteins are positive regulators or negative regulators (repressors), and one protein can have a dual role depending on the context (Johnson, P. F. and McKnight, S. L. Annu. Rev. Biochem. 58:799-839 (1989)).

One type of in-vitro assay utilizes a known DNA binding factor to isolate DNA fragments that bind. If a fragment or promoter variant does not bind, then a promoter control element has been removed or disrupted. For specific assays, see, e.g., B. Luo et al., J. Mol. Biol. 266:470 (1997), S. Chusacultanachai et al., J. Biol. Chem. 274:23591 (1999), D. Fabbro et al., Biochem. Biophys. Res. Comm. 213:781 (1995)).

Alternatively, a fragment of DNA suspected of conferring a particular pattern of specificity can be examined for activity in binding transcription factors involved in that specificity by methods such as DNA footprinting (e.g. D. J. Cousins et al., Immunology 99:101 (2000); V. Kolla et al., Biochem. Biophys. Res. Comm. 266:5 (1999)) or “mobility-shift” assays (E. D. Fabiani et al., J. Biochem. 347:147 (2000); N. Sugiura et al., J. Biochem 347:155 (2000)) or fluorescence polarization (e.g. Royer et al., U.S. Pat. No. 5,445,935). Both mobility shift and DNA footprinting assays can also be used to identify portions of large DNA fragments that are bound by proteins in unpurified transcription extracts prepared from tissues or organs of interest.

Cell-free transcription extracts can be prepared and used to directly assay in a reconstitutable system (Narayan et al., Biochemistry 39:818 (2000)).

In-Vivo Assays

Promoter control elements can be identified with reporter genes in in-vivo assays with the use of fragments of the instant promoters or variants of the instant promoter polynucleotides.

For example, various fragments can be inserted into a vector, comprising a basal promoter, for example, operably linked to a reporter sequence, which, when transcribed, can produce a detectable label. Examples of reporter genes include those encoding luciferase, green fluorescent protein, GUS, neo, cat and bar. Alternatively, reporter sequence can be detected utilizing AFLP and microarray techniques.

In promoter probe vector systems, genomic DNA fragments are inserted upstream of the coding sequence of a reporter gene that is expressed only when the cloned fragment contains DNA having transcription modulation activity (Neve, R. L. et al., Nature 277:324-325 (1979)). Control elements are disrupted when fragments or variants lacking any transcription modulation activity. Probe vectors have been designed for assaying transcription modulation in E. coli (An, G. et al., J. Bact. 140:400-407 (1979)) and other bacterial hosts (Band, L. et al., Gene 26:313-315 (1983); Achen, M. G., Gene 45:45-49 (1986)), yeast (Goodey, A. R. et al., Mol. Gen. Genet. 204:505-511 (1986)) and mammalian cells (Pater, M. M. et al., J. Mol. App. Gen. 2:363-371 (1984)).

A different design of a promoter/control element trap includes packaging into retroviruses for more efficient delivery into cells. One type of retroviral enhancer trap was described by von Melchner et al. (Genes Dev. 6(6):919-27 (1992); U.S. Pat. No. 5,364,783). The basic design of this vector includes a reporter protein coding sequence engineered into the U3 portion of the 3′ LTR. No splice acceptor consensus sequences are included, limiting its utility to work as an enhancer trap only. A different approach to a gene trap using retroviral vectors was pursued by Friedrich and Soriano (Genes Dev. 5(9):1513-23 (1991)), who engineered a lacZ-neo fusion protein linked to a splicing acceptor. LacZ-neo fusion protein expression from trapped loci allows not only for drug selection, but also for visualization of β-galatactosidase expression using the chromogenic substrate, X-gal.

A general review of tools for identifying transcriptional regulatory regions of genomic DNA is provided by J. W. Fickett et al. Curr. Opn. Biotechnol. 11:19 (2000).

(4) Non-Natural Control Elements

Non-natural control elements can be constructed by inserting, deleting or substituting nucleotides into the promoter control elements described above. Such control elements are capable of transcription modulation which can be determined using any of the assays described above.

E. Constructing Promoters with Control Elements

(1) Combining Promoters and Promoter Control Elements

The promoter polynucleotides and promoter control elements of the present invention, both naturally occurring and synthetic, can be combined with each other to produce the desired preferential transcription. Also, the polynucleotides of the invention can be combined with other known sequences to obtain other useful promoters to modulate, for example, tissue transcription specific or transcription specific to certain conditions. Such preferential transcription can be determined using the techniques or assays described above.

Fragments, variants, as well as full-length sequences such as those shown in SEQ ID NO:1, nucleotides 1-4582 of SEQ ID NO:4, nucleotides 1-3154 of SEQ ID NO:6 or nucleotides 1-1609 of SEQ ID NO:8 and relatives are useful alone or in combination.

The location and relation of promoter control elements within a promoter can affect the ability of the promoter to modulate transcription. The order and spacing of control elements is a factor when constructing promoters.

(2) Number of Promoter Control Elements

Promoters can contain any number of control elements. For example, a promoter can contain multiple transcription binding sites or other control elements. One element may confer tissue or organ specificity; another element may limit transcription to specific time periods, etc. Typically, promoters will contain at least a basal or core promoter as described above. Any additional element can be included as desired. For example, a fragment comprising a basal promoter can be fused with another fragment with any number of additional control elements.

(3) Spacing Between Control Elements

Spacing between control elements or the configuration or control elements can be determined or optimized to permit the desired protein-polynucleotide or polynucleotide interactions to occur.

For example, if two transcription factors bind to a promoter simultaneously or relatively close in time, the binding sites are spaced to allow each factor to bind without steric hindrance. The spacing between two such hybridizing control elements can be as small as a profile of a protein bound to a control element. In some cases, two protein binding sites can be adjacent to each other when the proteins bind at different times during the transcription process.

Further, when two control elements hybridize the spacing between such elements will be sufficient to allow the promoter polynucleotide to hairpin or loop to permit the two elements to bind. The spacing between two such hybridizing control elements can be as small as a t-RNA loop, to as large as 10 kb.

Typically, the spacing is no smaller than 5 bases; more typically, no smaller than 8; more typically, no smaller than 15 bases; more typically, no smaller than 20 bases; more typically, no smaller than 25 bases; even more typically, no more than 30, 35, 40 or 50 bases.

Usually, the fragment size in no larger than 5 kb bases; more usually, no larger than 2 kb; more usually, no larger than 1 kb; more usually, no larger than 800 bases; more usually, no larger than 500 bases; even more usually, no more than 250, 200, 150 or 100 bases.

Such spacing between promoter control elements can be determined using the techniques and assays described above.

F. Control of G564 or C541 Activity of Gene Expression

(1) Use Of Nucleic Acids of the Invention to Inhibit Gene Expression

The isolated sequences prepared as described herein, can be used to prepare expression cassettes useful in a number of techniques. For example, expression cassettes of the invention can be used to suppress endogenous G564 or C541 gene expression. Ihibiting expression can be useful, for instance, to modulate or prevent suspensor cell development and/or embryo size, shape and/or rate of development. Inhibition of expression is also useful for modulating fertility of a plant.

A number of methods can be used to inhibit gene expression in plants. For instance, antisense technology can be conveniently used. To accomplish this, a nucleic acid segment from the desired gene is cloned and operably linked to a promoter such that the antisense strand of RNA will be transcribed. The expression cassette is then transformed into plants and the antisense strand of RNA is produced. In plant cells, it has been suggested that antisense RNA inhibits gene expression by preventing the accumulation of mRNA which encodes the enzyme of interest, see, e.g., Sheehy et al., Proc. Nat. Acad. Sci. USA, 85:8805-8809 (1988), and Hiatt et al., U.S. Pat. No. 4,801,340.

The antisense nucleic acid sequence transformed into plants will be substantially identical to at least a portion of the endogenous suspensor-specific gene or genes to be repressed. The sequence, however, does not have to be perfectly identical to inhibit expression. The vectors of the present invention can be designed such that the inhibitory effect applies to other proteins within a family of genes exhibiting homology or substantial homology to the target gene.

For antisense suppression, the introduced sequence also need not be full length relative to either the primary transcription product or fully processed mRNA. Generally, higher homology can be used to compensate for the use of a shorter sequence. Furthermore, the introduced sequence need not have the same intron or exon pattern, and homology of non-coding segments may be equally effective. Normally, a sequence of between about 30 or 40 nucleotides and about full length nucleotides should be used, though a sequence of at least about 100 nucleotides is preferred, a sequence of at least about 200 nucleotides is more preferred, and a sequence of at least about 500 nucleotides is especially preferred.

Catalytic RNA molecules or ribozymes can also be used to inhibit expression of embryo-specific genes. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA-cleaving activity upon them, thereby increasing the activity of the constructs.

A number of classes of ribozymes have been identified. One class of ribozymes is derived from a number of small circular RNAs that are capable of self-cleavage and replication in plants. The RNAs replicate either alone (viroid RNAs) or with a helper virus (satellite RNAs). Examples include RNAs from avocado sunblotch viroid and the satellite RNAs from tobacco ringspot virus, lucerne transient streak virus, velvet tobacco mottle virus, solanum nodiflorum mottle virus and subterranean clover mottle virus. The design and use of target RNA-specific ribozymes is described in Haseloff et al. Nature, 334:585-591 (1988).

Another method of suppression is sense suppression. Introduction of expression cassettes in which a nucleic acid is configured in the sense orientation with respect to the promoter has been shown to be an effective means by which to block the transcription of target genes. For an example of the use of this method to modulate expression of endogenous genes see, Napoli et al., The Plant Cell 2:279-289 (1990), and U.S. Pat. Nos. 5,034,323, 5,231,020, and 5,283,184.

Generally, where inhibition of expression is desired, some transcription of the introduced sequence occurs. The effect may occur where the introduced sequence contains no coding sequence per se, but only intron or untranslated sequences homologous to sequences present in the primary transcript of the endogenous sequence. The introduced sequence generally will be substantially identical to the endogenous sequence intended to be repressed. This minimal identity will typically be greater than about 65%, but a higher identity might exert a more effective repression of expression of the endogenous sequences. Substantially greater identity of more than about 80% is preferred, though about 95% to absolute identity would be most preferred. As with antisense regulation, the effect should apply to any other proteins within a similar family of genes exhibiting homology or substantial homology.

For sense suppression, the introduced sequence in the expression cassette, needing less than absolute identity, also need not be full length, relative to either the primary transcription product or fully processed mRNA. This may be preferred to avoid concurrent production of some plants that are overexpressers. A higher identity in a shorter than full-length sequence compensates for a longer, less identical sequence. Furthermore, the introduced sequence need not have the same intron or exon pattern, and identity of non-coding segments will be equally effective. Normally, a sequence of the size ranges noted above for antisense regulation is used.

One of skill in the art will recognize that using technology based on specific nucleotide sequences (e.g., antisense or sense suppression technology), families of homologous genes can be suppressed with a single sense or antisense transcript. For instance, if a sense or antisense transcript is designed to have a sequence that is conserved among a family of genes, then multiple members of a gene family can be suppressed. Conversely, if the goal is to only suppress one member of a homologous gene family, then the sense or antisense transcript should be targeted to sequences with the most variance between family members.

Another means of inhibiting G564 or C541 function in a plant is by creation of dominant negative mutations. In this approach, non-functional, mutant G564 or C541 polypeptides, which retain the ability to interact with wild-type subunits are introduced into a plant.

(2) Use of Nucleic Acids of the Invention to Enhance Gene Expression

Isolated sequences prepared as described herein can also be used to prepare expression cassettes that enhance or increase endogenous G564 or C5541 gene expression. Where overexpression of a gene is desired, the desired gene from a different species may be used to decrease potential sense suppression effects. Enhanced expression of G564 or C541 polynucleotides is useful, for example, to modulate suspensor cell and/or embryo size, shape and/or rate of development. Enhanced expression is also useful for modulating plant fertility.

Any of a number of means well known in the art can be used to increase G564 or C541 activity in plants. Any organ can be targeted, such as shoot vegetative organs/structures (e.g. leaves, stems and tubers), roots, flowers and floral organs/structures (e.g. bracts, sepals, petals, stamens, carpels, anthers and ovules), seed (including apical or basal cells, suspensor, embryo, endosperm, and seed coat) and fruit. Alternatively, one or several G564 or C541 genes can be expressed constitutively (e.g., using the CaMV 35S promoter).

One of skill will recognize that the polypeptides encoded by the genes of the invention, like other proteins, have different domains that perform different functions. Thus, the gene sequences need not be full length, so long as the desired functional domain of the protein is expressed.

(3) Modification of endogenous G564 or C541 genes

Methods for introducing genetic mutations into plant genes and selecting plants with desired traits are well known. For instance, seeds or other plant material can be treated with a mutagenic chemical substance, according to standard techniques. Such chemical substances include, but are not limited to, the following: diethyl sulfate, ethylene imine, ethyl methanesulfonate and N-nitroso-N-ethylurea. Alternatively, ionizing radiation from sources such as, X-rays or gamma rays can be used.

Modified protein chains can also be readily designed utilizing various recombinant DNA techniques well known to those skilled in the art and described for instance, in Sambrook et al., supra. Hydroxylamine can also be used to introduce single base mutations into the coding region of the gene (Sikorski, et al., (1991). Meth. Enzymol. 194: 302-318). For example, the chains can vary from the naturally occurring sequence at the primary structure level by amino acid substitutions, additions, deletions, and the like. These modifications can be used in a number of combinations to produce the final modified protein chain.

Alternatively, homologous recombination can be used to induce targeted gene modifications by specifically targeting the G564 or C541 gene in vivo (see, generally, Grewal and Klar, Genetics 146: 1221-1238 (1997) and Xu et al., Genes Dev. 10: 2411-2422 (1996)). Homologous recombination has been demonstrated in plants (Puchta et al., Experientia 50: 277-284 (1994), Swoboda et al., EMBO J 13: 484-489 (1994); Offringa et al., Proc. Natl. Acad. Sci. USA 90: 7346-7350 (1993); and Kempin et al. Nature 389:802-803 (1997)).

In applying homologous recombination technology to the genes of the invention, mutations in selected portions of an G564 or C541 gene sequences (including 5′ upstream, 3′ downstream, and intragenic regions) such as those disclosed here are made in vitro and then introduced into the desired plant using standard techniques. Since the efficiency of homologous recombination is known to be dependent on the vectors used, use of dicistronic gene targeting vectors as described by Mountford et al., Proc. Natl. Acad. Sci. USA 91: 4303-4307 (1994); and Vaulont et al., Transgenic Res. 4: 247-255 (1995) are conveniently used to increase the efficiency of selecting for altered G564 or C541 gene expression in transgenic plants. The mutated gene will interact with the target wild-type gene in such a way that homologous recombination and targeted replacement of the wild-type gene will occur in transgenic plant cells, resulting in suppression of G564 or C541 activity.

Alternatively, oligonucleotides composed of a contiguous stretch of RNA and DNA residues in a duplex conformation with double hairpin caps on the ends can be used. The RNA/DNA sequence is designed to align with the sequence of the target G564 or C541 gene and to contain the desired nucleotide change. Introduction of the chimeric oligonucleotide on an extrachromosomal T-DNA plasmid results in efficient and specific G564 or C541 gene conversion directed by chimeric molecules in a small number of transformed plant cells. This method is described in Cole-Strauss et al., Science 273:1386-1389 (1996) and Yoon et al., Proc. Natl. Acad. Sci. USA 93: 2071-2076 (1996).

G. Heterologous Expression of the G564 or C541 Polynucleotides of the Invention

A DNA sequence coding for the desired polypeptide, for example a cDNA sequence encoding a full length protein, will preferably be combined with transcriptional and translational initiation regulatory sequences which will direct the transcription of the sequence from the gene in the intended tissues of the transformed plant.

For example, for overexpression, a plant promoter fragment may be employed which will direct expression of the gene in all tissues of a regenerated plant. Such promoters are referred to herein as “constitutive” promoters and are active under most environmental conditions and states of development or cell differentiation. Examples of constitutive promoters include the cauliflower mosaic virus (CaMV) 35S transcription initiation region, the 1′- or 2′- promoter derived from T-DNA of Agrobacterium tumafaciens, and other transcription initiation regions from various plant genes known to those of skill.

Alternatively, the plant promoter may direct expression of the polynucleotide of the invention in a specific tissue (tissue-specific promoters) or may be otherwise under more precise environmental control (inducible promoters). Examples of tissue-specific promoters under developmental control include promoters that initiate transcription only in certain tissues, such as fruit, seeds, or flowers. As noted above, the promoters from the G564 or C541 genes described here are particularly useful for directing gene expression so that a desired gene product is located in suspensor cells. Examples of environmental conditions that may affect transcription by inducible promoters include anaerobic conditions, elevated temperature, or the presence of light.

If proper polypeptide expression is desired, a polyadenylation region at the 3′-end of the coding region should be included. The polyadenylation region can be derived from the natural gene, from a variety of other plant genes, or from T-DNA.

The vector comprising the sequences (e.g., promoters or coding regions) from genes of the invention will typically comprise a marker gene which confers a selectable phenotype on plant cells. For example, the marker may encode biocide resistance, particularly antibiotic resistance, such as resistance to kanamycin, G418, bleomycin, hygromycin, or herbicide resistance, such as resistance to chlorosluforon or Basta.

G564 or C541 nucleic acid sequences of the invention are expressed recombinantly in plant cells to enhance and increase levels of endogenous G564 or C541 polypeptides. Alternatively, antisense or other G564 or C541 constructs (described above) are used to suppress G564 or C541 levels of expression. A DNA sequence coding for a G564 or C541 polypeptide, e.g., a cDNA sequence encoding a full length protein, can be combined with cis-acting (promoter) and trans-acting (enhancer) transcriptional regulatory sequences to direct the timing, tissue type and levels of transcription in the intended tissues of the transformed plant. Translational control elements can also be used.

The invention provides a G564 or C541 nucleic acid operably linked to a promoter that, in a preferred embodiment, is capable of driving the transcription of the G564 or C541 coding sequence in plants. The promoter can be, e.g., derived from plant or viral sources. The promoter can be, e.g., constitutively active, inducible, or tissue specific. In construction of recombinant expression cassettes, vectors, transgenics, of the invention, a different promoters can be chosen and employed to differentially direct gene expression, e.g., in some or all tissues of a plant or animal.

Typically, desired promoters are identified by analyzing the 5′ sequences of a genomic clone corresponding to the suspensor-specific genes described here. Sequences characteristic of promoter sequences can be used to identify the promoter. Sequences controlling eukaryotic gene expression have been extensively studied. For instance, promoter sequence elements include the TATA box consensus sequence (TATAAT), which is usually 20 to 30 base pairs upstream of the transcription start site. In most instances the TATA box is required for accurate transcription initiation. In plants, further upstream from the TATA box, at positions -80 to -100, there is typically a promoter element with a series of adenines surrounding the trinucleotide G (or T) N G. J. Messing et aL, in genetic engineering in plants, pp.221-227 (Kosage, Meredith and Hollaender, eds. (1983)). A number of methods are known to those of skill in the art for identifying and characterizing promoter regions in plant genomic DNA (see, e.g., Jordano, et al., Plant Cell, 1: 855-866 (1989); Bustos, et al., Plant Cell, 1:839-854 (1989); Green, et al., EMBO J. 7, 4035-4044 (1988); Meier, et al., Plant Cell, 3, 309-316 (1991); and Zhang (1996) Plant Physiology 110:1069-1079).

Constitutive Promoters

A promoter fragment can be employed which will direct expression of G564 or C541 nucleic acid in all transformed cells or tissues, e.g. as those of a regenerated plant. Such promoters are referred to herein as “constitutive” promoters and are active under most environmental conditions and states of development or cell differentiation. Promoters that drive expression continuously under physiological conditions are referred to as “constitutive” promoters and are active under most environmental conditions and states of development or cell differentiation. Examples of constitutive promoters include those from viruses which infect plants, such as the cauliflower mosaic virus (CaMV) 35S transcription initiation region (see, e.g., Dagless (1997) Arch. Virol. 142:183-191); the 1′- or 2′- promoter derived from T-DNA of Agrobacterium tumafaciens (see, e.g., Mengiste (1997) supra; O'Grady (1995) Plant Mol. Biol. 29:99-108); the promoter of the tobacco mosaic virus; the promoter of Figwort mosaic virus (see, e.g., Maiti (1997) Transgenic Res. 6:143-156); actin promoters, such as the Arabidopsis actin gene promoter (see, e.g., Huang (1997) Plant Mol. Biol. 1997 33:125-139); alcohol dehydrogenase (Adh) gene promoters (see, e.g., Millar (1996) Plant Mol. Biol. 31:897-904); ACT11 from Arabidopsis (Huang et al. Plant Mol. Biol. 33:125-139 (1996)), Cat3 from Arabidopsis (GenBank No. U43147, Zhong et al., Mol. Gen. Genet. 251:196-203 (1996)), the gene encoding stearoyl-acyl carrier protein desaturase from Brassica napus (Genbank No. X74782, Solocombe et al. Plant Physiol. 104:1167-1176 (1994)), GPcl from maize (GenBank No. X15596, Martinez et al. J. Mol. Biol 208:551-565 (1989)), Gpc2 from maize (GenBank No. U45855, Manjunath et al., Plant Mol. Biol. 33:97-112 (1997)), other transcription initiation regions from various plant genes known to those of skill. See also Holtorf (1995) “Comparison of different constitutive and inducible promoters for the overexpression of transgenes in Arabidopsis thaliana,” Plant Mol. Biol. 29:637-646.

Inducible Promoters

Alternatively, a plant promoter may direct expression of the G564 or C541 nucleic acids of the invention under the influence of changing environmental conditions or developmental conditions. Examples of environmental conditions that may effect transcription by inducible promoters include anaerobic conditions, elevated temperature, drought, or the presence of light. Such promoters are referred to herein as “inducible” promoters. For example, the invention incorporates the drought-inducible promoter of maize (Busk (1997) supra); the cold, drought, and high salt inducible promoter from potato (Kirch (1997) Plant Mol. Biol. 33:897-909).

Alternatively, plant promoters which are inducible upon exposure to plant hormones, such as auxins, are used to express the nucleic acids of the invention. For example, the invention can use the auxin-response elements E1 promoter fragment (AuxREs) in the soybean (Glycine max L.) (Liu (1997) Plant Physiol. 115:397-407); the auxin-responsive Arabidopsis GST6 promoter (also responsive to salicylic acid and hydrogen peroxide) (Chen (1996) Plant J. 10: 955-966); the auxin-inducible parC promoter from tobacco (Sakai (1996) 37:906-913); a plant biotin response element (Streit (1997) Mol. Plant Microbe Interact. 10:933-937); and, the promoter responsive to the stress hormone abscisic acid (Sheen (1996) Science 274:1900-1902).

Plant promoters which are inducible upon exposure to chemicals reagents which can be applied to the plant, such as herbicides or antibiotics, are also used to express the nucleic acids of the invention. For example, the maize In2-2 promoter, activated by benzenesulfonamide herbicide safeners, can be used (De Veylder (1997) Plant Cell Physiol. 38:568-577); application of different herbicide safeners induces distinct gene expression patterns, including expression in the root, hydathodes, and the shoot apical meristem. The G564 or C541 coding sequences can also be under the control of, e.g., a tetracycline-inducible promoter, e.g., as described with transgenic tobacco plants containing the Avena sativa L. (oat) arginine decarboxylase gene (Masgrau (1997) Plant J. 11:465-473); or, a salicylic acid-responsive element (Stange (1997) Plant J. 11:1315-1324.

The following are promoters that are induced under stress conditions and can be combined with those of the present invention: ldhl (oxygen stress; tomato; see Germain and Ricard Plant Mol Biol 35:949-54 (1997)), GPx and CAT (oxygen stress; mouse; see Franco et al. Free Radic Biol Med 27:1122-32 (1999), ci7 (cold stress; potato; see Kirch et al. Plant Mol Biol. 33:897-909 (1997)), Bz2 (heavy metals; maize; see Marrs and Walbot. Plant Physiol 113:93-102 (1997)), HSP32 (hyperthermia; rat; see Raju and Maines. Biochim Biophys Acta 1217:273-80 (1994)); MAPKAPK-2 (heat shock; Drosophila; see Larochelle and Suter Gene 163:209-14 (1995)).

In addition, the following examples of promoters are induced by the presence or absence of light can be used in combination with those of the present invention: Topoisomerase II (pea; see Reddy et al. Plant Mol Biol 41:125-37 (1999)), chalcone synthase (soybean; see Wingender et al. Mol Gen Genet 218:315-22 (1989)) mdm2 gene (human tumor; see Saucedo et al. Cell Growth Differ 9:119-30 (1998)), Clock and BMAL1 (rat; see Namihira et al. Neurosci Lett 271:1-4 (1998), PHYA (Arabidopsis; see Canton and Quail Plant Physiol 121:1207-16 (1999)), PRB-lb (tobacco; see Sessa et al. Plant Mol Biol 28:537-47 (1995)) and YprlO (common bean; see Walter et al. Eur J Biochem 239:281-93 (1996)).

Tissue-Specific Promoters

Alternatively, the plant promoter may direct expression of the polynucleotide of the invention in a specific tissue (tissue-specific promoters). Tissue specific promoters are transcriptional control elements that are only active in particular cells or tissues at specific times during plant development, such as in vegetative tissues or reproductive tissues. Promoters from the G564 or C541 genes of the invention are particularly useful for tissue-specific direction of gene expression so that a desired gene product is generated only or preferentially in suspensors, as described below.

Examples of tissue-specific promoters under developmental control include promoters that initiate transcription only (or primarily only) in certain tissues, such as vegetative tissues, e.g., roots or leaves, or reproductive tissues, such as fruit, ovules, seeds, pollen, pistols, flowers, or any embryonic tissue. Reproductive tissue-specific promoters may be, e.g., ovule-specific, embryo-specific, endosperm-specific, integument-specific, seed and seed coat-specific, pollen-specific, petal-specific, sepal-specific, or some combination thereof.

Suitable seed-specific promoters are derived from the following genes: MAC1 from maize, Sheridan (1996) Genetics 142:1009-1020; Cat3 from maize, GenBank No. L05934, Abler (1993) Plant Mol. Biol. 22:10131-1038; vivparous-1 from Arabidopsis, Genbank No. U93215; atmycI from Arabidopsis, Urao (1996) Plant Mol. Biol. 32:571-57; Conceicao (1994) Plant 5:493-505; napA from Brassica napus, GenBank No. J02798, Josefsson (1987) JBL 26:12196-1301; the napin gene family from Brassica napus, Sjodahl (1995) Planta 197:264-271.

The ovule-specific BELl gene described in Reiser (1995) Cell 83:735-742, GenBank No. U39944, can also be used. See also Ray (1994) Proc. Natl. Acad. Sci. USA 91:5761-5765. The egg and central cell specific FIE1 promoter is also a useful reproductive tissue-specific promoter.

Sepal and petal specific promoters are also used to express G564 nucleic acids in a reproductive tissue-specific manner. For example, the Arabidopsis floral homeotic gene APETALA1 (AP1) encodes a putative transcription factor that is expressed in young flower primordia, and later becomes localized to sepals and petals (see, e.g., Gustafson- Brown (1994) Cell 76:131-143; Mandel (1992) Nature 360:273-277). A related promoter, for AP2, a floral homeotic gene that is necessary for the normal development of sepals and petals in floral whorls, is also useful (see, e.g., Drews (1991) Cell 65:991-1002; Bowman (1991) Plant Cell 3:749-758). Another useful promoter is that controlling the expression of the unusual floral organs (ufo) gene of Arabidopsis, whose expression is restricted to the junction between sepal and petal primordia (Bossinger (1996) Development 122:1093-1102).

A maize pollen-specific promoter has been identified in maize (Guerrero (1990) Mol. Gen. Genet. 224:161-168). Other genes specifically expressed in pollen are described, e.g., by Wakeley (1998) Plant Mol. Biol. 37:187-192; Ficker (1998) Mol. Gen. Genet. 257:132-142; Kulikauskas (1997) Plant Mol. Biol. 34:809-814; Treacy (1997) Plant Mol. Biol. 34:603-611.

Other suitable promoters include those from genes encoding embryonic storage proteins. For example, the gene encoding the 2S storage protein from Brassica napus, Dasgupta (1993) Gene 133:301-302; the 2s seed storage protein gene family from Arabidopsis; the gene encoding oleosin 2OkD from Brassica napus, GenBank No. M63985; the genes encoding oleosin A, Genbank No. U09118, and, oleosin B, Genbank No. U09119, from soybean; the gene encoding oleosin from Arabidopsis, Genbank No. Z17657; the gene encoding oleosin 18 kD from maize, GenB ank No. J05212, Lee (1994) Plant Mol. Biol. 26:1981-1987; and, the gene encoding low molecular weight sulphur rich protein from soybean, Choi (1995) Mol Gen, Genet. 246:266-268, can be used. The tissue specific E8 promoter from tomato is particularly useful for directing gene expression so that a desired gene product is located in fruits.

A tomato promoter active during fruit ripening, senescence and abscission of leaves and, to a lesser extent, of flowers can be used (Blume (1997) Plant J. 12:731-746). Other exemplary promoters include the pistol specific promoter in the potato (Solanum tuberosum L.) SK2 gene, encoding a pistil-specific basic endochitinase (Ficker (1997) Plant Mol. Biol. 35:425-431); the Blec4 gene from pea (Pisum sativum cv. Alaska), active in epidermal tissue of vegetative and floral shoot apices of transgenic alfalfa. This makes it a useful tool to target the expression of foreign genes to the epidermal layer of actively growing shoots.

A variety of promoters specifically active in vegetative tissues, such as leaves, stems, roots and tubers, can also be used to express the G564 or C541 nucleic acids of the invention. For example, promoters controlling patatin, the major storage protein of the potato tuber, can be used, see, e.g., Kim (1994) Plant Mol. Biol. 26:603-615; Martin (1997) Plant J. 11:53-62. The ORF13 promoter from Agrobacterium rhizogenes which exhibits high activity in roots can also be used (Hansen (1997) Mol. Gen. Genet. 254:337-343. Other useful vegetative tissue-specific promoters include: the tarin promoter of the gene encoding a globulin from a major taro (Colocasia esculenta L. Schott) corm protein family, tarin (Bezerra (1995) Plant Mol. Biol. 28:137-144); the curculin promoter active during taro corm development (de Castro (1992) Plant Cell 4:1549-1559) and the promoter for the tobacco root-specific gene TobRB7, whose expression is localized to root meristem and immature central cylinder regions (Yamamoto (1991) Plant Cell 3:371-382).

Leaf-specific promoters, such as the ribulose biphosphate carboxylase (RBCS) promoters can be used. For example, the tomato RBCS1, RBCS2 and RBCS3A genes are expressed in leaves and light-grown seedlings, only RBCS1 and RBCS2 are expressed in developing tomato fruits (Meier (1997) FEBS Lett. 415:91-95). A ribulose bisphosphate carboxylase promoters expressed almost exclusively in mesophyll cells in leaf blades and leaf sheaths at high levels, described by Matsuoka (1994) Plant J. 6:311-319, can be used. Another leaf-specific promoter is the light harvesting chlorophyll a/b binding protein gene promoter, see, e.g., Shiina (1997) Plant Physiol. 115:477-483; Casal (1998) Plant Physiol. 116:1533-1538. The Arabidopsis thaliana myb-related gene promoter (Atmyb5) described by Li (1996) FEBS Lett. 379:117-121, is leaf-specific. The Atmyb5 promoter is expressed in developing leaf trichomes, stipules, and epidermal cells on the margins of young rosette and cauline leaves, and in immature seeds. Atmyb5 mRNA appears between fertilization and the 16 cell stage of embryo development and persists beyond the heart stage. A leaf promoter identified in maize by Busk (1997) Plant J. 11:1285-1295, can also be used.

Another class of useful vegetative tissue-specific promoters are meristematic (root tip and shoot apex) promoters. For example, the “SHOOTMERISTEMLESS” and “SCARECROW” promoters, which are active in the developing shoot or root apical meristems, described by Di Laurenzio (1996) Cell 86:423-433; and, Long (1996) Nature 379:66-69; can be used. Another useful promoter is that which controls the expression of 3-hydroxy-3- methylglutaryl coenzyme A reductase HMG2 gene, whose expression is restricted to meristematic and floral (secretory zone of the stigma, mature pollen grains, gynoecium vascular tissue, and fertilized ovules) tissues (see, e.g., Enjuto (1995) Plant Cell. 7:517-527). Also useful are knl-related genes from maize and other species which show meristem-specific expression, see, e.g., Granger (1996) Plant Mol. Biol. 31:373-378; Kerstetter (1994) Plant Cell 6:1877-1887; Hake (1995) Philos. Trans. R. Soc. Lond. B. Biol. Sci. 350:45-51. For example, the Arabidopsis thaliana KNAT1 promoter. In the shoot apex, KNAT1 transcript is localized primarily to the shoot apical meristem; the expression of KNAT1 in the shoot meristem decreases during the floral transition and is restricted to the cortex of the inflorescence stem (see, e.g., Lincoln (1994) Plant Cell 6:1859-1876).

One of skill will recognize that a tissue-specific promoter may drive expression of operably linked sequences in tissues other than the target tissue. Thus, as used herein a tissue-specific promoter is one that drives expression preferentially in the target tissue, but may also lead to some expression in other tissues as well.

In another embodiment, a G564 nucleic acid is expressed through a transposable element. This allows for constitutive, yet periodic and infrequent expression of the constitutively active polypeptide. The invention also provides for use of tissue-specific promoters derived from viruses which can include, e.g., the tobamovirus subgenomic promoter (Kumagai (1995) Proc. Natl. Acad. Sci. USA 92:1679-1683; the rice tungro bacilliform virus (RTBV), which replicates only in phloem cells in infected rice plants, with its promoter which drives strong phloem-specific reporter gene expression; the cassava vein mosaic virus (CVMV) promoter, with highest activity in vascular elements, in leaf mesophyll cells, and in root tips (Verdaguer (1996) Plant Mol. Biol. 31:1129-1139).

The promoters and control elements of the following genes can also be used in combination with the present invention to confer tissue specificity: MipB (iceplant; Yamada et al. Plant Cell 7:1129-42 (1995)) and SUCS (root nodules; broadbean; Kuster et al. Mol Plant Microbe Interact 6:507-14 (1993)) for roots, OsSUT1 (rice; Hirose et al. Plant Cell Physiol 38:1389-96 (1997)) for leaves, Msg (soybean; Stomvik et al. Plant Mol Biol 41:217-31(1999)) for siliques, cell (Arabidopsis; Shani et al. Plant Mol Biol 34(6):837-42 (1997)) and ACT11 (Arabidopsis; Huang et al. Plant Mol Biol 33:125-39 (1997)) for inflorescence.

Still other promoters are affected by hormones or participate in specific physiological processes, which can be used in combination with those of present invention. Some examples are the ACC synthase gene that is induced differently by ethylene and brassinosteroids (mung bean; Yi et al. Plant Mol Biol 41:443-54 (1999)), the TAPG1 gene that is active during abscission (tomato; Kalaitzis et al. Plant Mol Biol 28:647-56 (1995)), and the 1-aminocyclopropane-1-carboxylate synthase gene (carnation; Jones et al. Plant Mol Biol 28:505-12 (1995)) and the CP-2/cathepsin L gene (rat; Kim and Wright. Biol Reprod 57:1467-77 (1997)), both active during senescence.

H. Vectors

Vectors are a useful component of the present invention. In particular, the present promoters and/or promoter control elements may be delivered to a system such as a cell by way of a vector. For the purposes of this invention, such delivery may range from simply introducing the promoter or promoter control element by itself randomly into a cell to integration of a cloning vector containing the present promoter or promoter control element. Thus, a vector need not be limited to a DNA molecule such as a plasmid, cosmid or bacterial phage that has the capability of replicating autonomously in a host cell. All other manner of delivery of the promoters and promoter control elements of the invention are envisioned. The various T-DNA vector types are a preferred vector for use with the present invention. Many useful vectors are commercially available.

It may also be useful to attach a marker sequence to the present promoter and promoter control element in order to determine activity of such sequences. Marker sequences typically include genes that provide antibiotic resistance, such as tetracycline resistance, hygromycin resistance or ampicillin resistance, or provide herbicide resistance. Specific selectable marker genes may be used to confer resistance to herbicides such as glyphosate, glufosinate or broxynil (Comai et al., Nature 317: 741-744 (1985); Gordon-Kamm et al., Plant Cell 2: 603-618 (1990); and Stalker et al., Science 242: 419-423 (1988)). Other marker genes exist which provide hormone responsiveness.

(1) Modification of Transcription bv Promoters and Promoter Control Elements

The promoter or promoter control element of the present invention may be operably linked to a polynucleotide to be transcribed. In this manner, the promoter or promoter control element may modify transcription by modulate transcript levels of that polynucleotide when inserted into a genome.

However, prior to insertion into a genome, the promoter or promoter control element need not be linked, operably or otherwise, to a polynucleotide to be transcribed. For example, the promoter or promoter control element may be inserted alone into the genome in front of a polynucleotide already present in the genome. In this manner, the promoter or promoter control element may modulate the transcription of a polynucleotide that was already present in the genome. This polynucleotide may be native to the genome or inserted at an earlier time.

Alternatively, the promoter or promoter control element may be inserted into a genome alone to modulate transcription. See, for example, Vaucheret, H et al. (1998) Plant J 16: 651-659. Rather, the promoter or promoter control element may be simply inserted into a genome or maintained extrachromosomally as a way to divert transcription resources of the system to itself. This approach may be used to down-regulate the transcript levels of a group of polynucleotide(s).

(2) Polynucleotides to be Transcribed

The nature of the polynucleotide to be transcribed is not limited. Specifically, the polynucleotide may include sequences which will have activity as RNA as well as sequences which result in a polypeptide product. These sequences may include, but are not limited to antisense sequences, ribozyme sequences, spliceosomes, amino acid coding sequences, and fragments thereof.

Specific coding sequences may include, but are not limited to endogenous proteins or fragments thereof, or heterologous proteins including marker genes or fragments thereof.

Promoters and control elements of the present invention are useful for modulating metabolic or catabolic processes. Such processes include, but are not limited to, secondary product metabolism, amino acid synthesis, seed protein storage, oil development, pest defense and nitrogen usage. Some examples of genes, transcripts and peptides or polypeptides participating in these processes, which can be modulated by the present invention: are tryptophan decarboxylase (tdc) and strictosidine synthase (strl), dihydrodipicolinate synthase (DHDPS) and aspartate kinase (AK), 2S albumin and alpha-, beta-, and gamma-zeins, ricinoleate and 3-ketoacyl-ACP synthase (KAS), Bacillus thuringiensis (Bt) insecticidal protein, cowpea trypsin inhibitor (CpTI), asparagine synthetase and nitrite reductase. Alternatively, expression constructs can be used to inhibit expression of these peptides and polypeptides by incorporating the promoters in constructs for antisense use, co-suppression use or for the production of dominant negative mutations.

(3) Other Regulatory Elements

As explained above, several types of regulatory elements exist concerning transcription regulation. Each of these regulatory elements may be combined with the present vector if desired.

(4) Other Components of Vectors

Translation of eukaryotic mRNA is often initiated at the codon which encodes the first methionine. Thus, when constructing a recombinant polynucleotide according to the present invention for expressing a protein product, it is preferable to ensure that the linkage between the 3′ portion, preferably including the TATA box, of the promoter and the polynucleotide to be transcribed, or a functional derivative thereof, does not contain any intervening codons which are capable of encoding a methionine.

The vector of the present invention may contain additional components. For example, an origin of replication allows for replication of the vector in a host cell. Additionally, homologous sequences flanking a specific sequence allows for specific recombination of the specific sequence at a desired location in the target genome. T-DNA sequences also allow for insertion of a specific sequence randomly into a target genome.

The vector may also be provided with a plurality of restriction sites for insertion of a polynucleotide to be transcribed as well as the promoter and/or promoter control elements of the present invention. The vector may additionally contain selectable marker genes. The vector may also contain a transcriptional and translational initiation region, and a transcriptional and translational termination region functional in the host cell. The termination region may be native with the transcriptional initiation region, may be native with the polynucleotide to be transcribed, or may be derived from another source. Convenient termination regions are available from the Ti-plasmid of A. tumefaciens, such as the octopine synthase and nopaline synthase termination regions. See also, Guerineau et al., Mol. Gen. Genet. 262:141-144 (199 1); Proudfoot, Cell 64:671-674 (1991); Sanfacon et al., Genes Dev. 5:141-149 (1991); Mogen et al., Plant Cell 2:1261-1272 (1990); Munroe et al., Gene 91:151-158 (1990); Ballas et al., Nucleic Acids Res. 17:7891-7903 (1989); Joshi et al., Nucleic Acid Res. 15:9627-9639 (1987).

Where appropriate, the polynucleotide to be transcribed may be optimized for increased expression in a certain host cell. For example, the polynucleotide can be synthesized using preferred codons for improved transcription and translation. See U.S. Pat. Nos. 5,380,831, 5,436, 391; see also Murray et al, Nucleic Acids Res. 17:477-498 (1989).

Additional sequence modifications include elimination of sequences encoding spurious polyadenylation signals, exon intron splice site signals, transposon-like repeats, and other such sequences well characterized as deleterious to expression. The G-C content of the polynucleotide may be adjusted to levels average for a given cellular host, as calculated by reference to known genes expressed in the host cell. The polynucleotide sequence may be modified to avoid hairpin secondary mRNA structures.

A general description of expression vectors and reporter genes can be found in Gruber, et al., “Vectors for Plant Transformation, in Methods in Plant Molecular Biology & Biotechnology” in methods in plant molecular biology & biotechnology, (Glich et al., eds. 1993) pp. 89-119. Moreover GUS expression vectors and GUS gene cassettes are available from Clonetech Laboratories, Inc., Palo Alto, Calif. while luciferase expression vectors and luciferase gene cassettes are available from Promega Corp. (Madison, Wis.). GFP vectors are available from Aurora Biosciences.

I. Polynucleotide Insertion Into A Host Cell

The polynucleotides according to the present invention can be inserted into a host cell. A host cell includes but is not limited to a plant, mammalian, insect, yeast, and prokaryotic cell, preferably a plant cell.

The method of insertion into the host cell genome is choosen based on convenience. For example, the insertion into the host cell genome may either be accomplished by vectors which integrate into the host cell genome or by vectors which exist independent of the host cell genome.

The nucleic acids of the invention can be used to confer desired traits on essentially any plant. Thus, the invention has use over a broad range of plants, including species from the genera Asparagus, Atropa, Avena, Brassica, Citrus, Citrullus, Capsicum, Cucumis, Cucurbita, Daucus, Fragaria, Glycine, Gossypium, Helianthus, Heterocallis, Hordeum, Hyoscyamus, Lactuca, Linum, Lolium, Lycopersicon, Malus, Manihot, Majorana, Medicago, Nicotiana, Oryza, Panieum, Pannesetum, Persea, Pisum, Pyrus, Prunus, Raphanus, Secale, Senecio, Sinapis, Solanum, Sorghum, Trigonella, Triticum, Vitis, Vigna, and, Zea.

(1) Polynucleotides Autonomous of the Host Genome

The polynucleotides the present invention can exist autonomous or independent of the host cell genome. Vectors of these types are known in the art and include, for example, certain type of non-integrating viral vectors, autonomously replicating plasmids, artificial chromosomes, and the like.

Additionally, in some cases transient expression of a polynucleotide may be desired.

(2) Polynucleotides Integrated into the Host Genome

The promoter sequences, promoter control elements or vectors of the present invention may be transformed into host cells. These transformations may be into protoplasts or intact tissues or isolated cells. Preferably expression vectors are introduced into intact tissue. General methods of culturing plant tissues are provided for example by Maki et al. “Procedures for Introducing Foreign DNA into Plants” in methods in plant molecular biology & biotechnology, (Glich et al., eds. 1993) pp. 67-88; and by Phillips et al. “Cell-Tissue Culture and In-Vitro Manipulation” in corn & corn improvement, 3rd Edition (Sprague et al., eds. 1998) pp. 345-387.

Methods of introducing polynucleotides into plant tissue include the direct infection or co-cultivation of plant cell with Agrobacterium tumefaciens, Horsch et al., Science, 227:1229 (1985). Descriptions of Agrobacterium vector systems and methods for Agrobacterium-mediated gene transfer provided by Gruber et al. supra.

Alternatively, polynucleotides are introduced into plant cells or other plant tissues using a direct gene transfer method such as microprojectile-mediated delivery, DNA injection, electroporation and the like. More preferably polynucleotides are introduced into plant tissues using the microprojectile media delivery with the biolistic device. See, for example, Tomes et al., “Direct DNA transfer into intact plant cells via microprojectile bombardment” in plant cell, tissue and organ culture: fundamental methods (:Gamborg and Phillips, eds. 1995).

In another embodiment of the current invention, expression constructs can be used for gene expression in callus culture for the purpose of expressing marker genes encoding peptides or polypeptides which allow identification of transformed plants. Here, a promoter that is operatively linked to a polynucleotide to be transcribed is transformed into plant cells and the transformed tissue is then placed on callus-inducing media. If the transformation is conducted with leaf discs, for example, callus will initiate along the cut edges. Once callus growth has initiated, callus cells can be transferred to callus shoot-inducing or callus root-inducing media. Gene expression will occur in the callus cells developing on the appropriate media: callus root-inducing promoters will be activated on callus root-inducing media, etc. Examples of such peptides or polypeptides useful as transformation markers include, but are not limited to barstar, glyphosate, chloramphenicol acetyltransferase (CAT), kanamycin, spectinomycin, streptomycin or other antibiotic resistance enzymes, green fluorescent protein (GFP), and β-glucuronidase (GUS), etc. Some of the promoters of the invention will also be capable of sustaining expression in some tissues or organs after the initiation or completion of regeneration. Examples of these tissues or organs are somatic embryos, cotyledon, hypocotyl, epicotyl, leaf, stems, roots, flowers and seed.

Integration into the host cell genome also can be accomplished by methods known in the art, for example, by the homologous sequences or T-DNA discussed above or using the cre-lox system (A. C. Vergunst et al., Plant Mol. Biol. 38:393 (1998)).

J. Utility

Common Uses

The polynucleotides of the invention have a variety of uses. For example, modulation of expression of the gene products of the invention can be used to modulate suspensor cell and/or embryo size, shape or rates of development.

The suspensor-specific promoters of the invention are also useful for expression of any number of polynucleotides in a suspensor-specific fashion. Exemplary gene products that can be expressed under the control of the promoters of the invention include toxic gene products. In some embodiments, toxic gene products are also expressed in the embryo under the control of the same or a second promoter. By preventing development of the suspensor cell and/or the embryo, plants with modulated fertility and/or that produce seedless fruit can be developed.

Examples of toxic genes include, e.g., those which produce toxic substances, disrupt cell function, suppress genes required by the cell (such as by using anti-sense, sense suppression, or ribozymes), and disruption of mitochondrial finction. Particular examples include, barnase (Sancho & Fersht, J. Mol. Biol. 224:741-47 (1992)). diphtheria toxin (DT) A chain, which adenoribosylates elongation factor EF-2, thus blocking protein synthesis (Herrera et al., Proc. NatL. Acad. Sci., USA 91:12999-13003 (1994)), and the thymidine kinase (tk) gene, which provides a conditional cell-lethal finction, requiring the presence of a nucleoside analog such as ganciclovir for lethality (Brady et al., Proc. Natl. Acad. Sci., USA 91:365-69 (1994)).

Alternatively, growth regulators such as gene products that modulate gibberellin expression, can be specifically expressed within the suspensor, thereby modulating (e.g., increasing or decreasing) the attached embryo's size, shape of rate of development.

An additional utility includes the expression of gene products that induce embryonic features to the suspensor cell, thereby leading to the development of a second embryo. Examples of the gene products that induce embryonic features include the LEC1 (see, e.g., Lotan, et al. Cell 93(7):1195-205 (1998)).

In yet another use, nucleic acids of the invention can be used in the development of apomictic plant lines (i.e., plants in which asexual reproductive processes occur in the ovule, see, Koltunow, A. Plant Cell 5: 1425-1437 (1993) for a discussion of apomixis). Apomixis provides a novel means to select and fix complex heterozygous genotypes that cannot be easily maintained by traditional breeding. Thus, for instance, new hybrid lines with desired traits (e.g., hybrid vigor) can be obtained and readily maintained.

In yet another use, expression cassettes comprising the promoter polynucleotides of the invention can be used to express genes that result in apomictic plants. Examples of genes useful in creating apomictic planst include LEC1 nucleic acids as described by Lotan, et al. Cell 93: 1195-1205 (1998) and in USSN 09/026,221 as well as FIE and MEDEA nucleic acids as described in Ohad et al., Plant Cell 11:407-415 (1999); Grossniklaus et al., Science 280:446-450 (1998) and USSN 09/177,249. In these embodiments, constructs providing expression of a LEC 1, FIE, MEDEA or other nucleic acids capable of inducing apomictic fruit are used alone or in combination.

The following examples are provided for a further understanding of the invention, however, the invention is not to be construed as limited thereto.

### EXAMPLES

### MATERIALS AND METHODS

Plant materials and maintenance

Seeds of the day neutral Scarlet Runner Bean cultivar ‘Hammond's Dwarf Red Flower’ (Vermont Bean Seed Company, Fair Haven, Vt.; Nagl, 1990) were germinated in a soil mixture of vermiculite, perlite, sandy-loam soil, sphagnum peat moss, and plaster sand respectively at a ratio of 3:3:2:2:2. Plants were maintained in a 16:8 hour light/dark cycle in the greenhouse. Flowers were hand-pollinated by lightly brushing the stigma with a watercolor brush containing pollen. Hand-pollinated flowers were tagged and seeds were harvested at specific days after pollination.

Suspensor isolation

The micropylar half of a 6 days after pollination (DAP) seed was cut and placed upright on its cut side under a dissecting microscope. Approximately 1 mm was sliced from the left and right sides of the seed coat “flat face.” The seed was turned on its “flat face” and the remaining seed coat and endosperm were removed from the exposed embryo proper. The entire embryo was isolated and then the suspensor was separated from the embryo proper by microdissection. Generally, ten suspensors were isolated per hour.

RNA isolation and gel blot analysis

Polysomal RNAs were isolated according to the procedure of Cox and Goldberg (1988). Poly(A) mRNA was isolated from total polysomal RNA using the PolyATract® mRNA isolation system (Promega: Madison, Wis.) and the protocol supplied by the manufacturer. Total RNAs, used for the Differential Display Reverse Transcription Polymerase Chain Reaction (DD-RT-PCR) and RNA gel blot experiments, were isolated using the RNAeasy® plant total RNA kit (Qiagen: Chatsworth, Calif.). RNAs were treated with RNAse-free DNAse (Boehringer Manaheim: Indianapolis, Ind.) following the protocol of Ausubel et al. (1992). RNA gel blots were carried out as described by Sambrook et al. (1989). ³²P-labeled DNA probes for the RNA gel blots were prepared by the random-priming procedure of Feinberg and Vogelstein (1984).

cDNA library construction and screening

A cDNA library of 5-9 DAP Scarlet Runner Bean seeds containing globular-stage embryos was constructed using the ZAP Express® cDNA synthesis kit (Stratagene: La Jolla, Calif.). Poly(A) mRNA was used as a template to generate first-strand cDNA using MMLV reverse transcriptase and a 50-base oligonucleotide linker-primer [5′-(GA)IoACTAGTCTCGAG(T)₁₈ -3′]. Double-strand cDNAs were blunt-ended and ligated to an EcoRi adapter. After phosphorylation of EcoRI 5′ ends, the cDNAs were digested with XhoI and size-fractionated on a Sephacryl S-400 column to exclude cDNAs that were smaller than 250 bp. The fractionated cDNAs were ligated to the λZAP vector. About 3,000 10 recombinants from the unamplified library were differentially screened with ³²P-labeled first- strand cDNAs generated from: (1) 5-9 DAP seed micropylar region poly(A) mRNA and (2) leaf poly(A) mRNA. cDNA clones representing mRNAs preferentially present in the micropylar region were screened two more times following the strategy used in the primary screen.

Differential display reverse transcription polymerase chain reaction

Differential display procedures of Liang and Pardee (Liang, P., et al., Science, 257:967-971 (1992)) were followed using the RNAimage™ kit (GenHunter Corp.:

Nashville, Tenn.). Differential display reactions were carried out using total RNA templates from: (1) 6-8 DAP dissected suspensors of globular-stage embryos, (2) 6 DAY embryo-containing micropylar seed regions, (3) 6 DAP non-embryo-containing chalazal seed regions, (4) 6-8 DAP isolated globular-stage embryo propers, (5) leaves, (6) ovules, (7) 2 DAY whole seeds, and (8) 3 DAP whole seeds. Briefly, first-strand cDNAs were generated by reverse transcription (RT) of 200 ng of total RNA using MMLV reverse transcriptase and an anchor/reverse primer (G primer: 5′-AAGCTIG-3′ or C primer: 5′-AAGCT₁₁C-3′). Aliquots of the first-strand cDNAs were used as templates for the polymerase chain reaction (PCR) using combinations of forward and anchor/reverse primers in the presence of ³³P-dCTP and AmpliTaq® polymerase (Perkin Elmer; Branchburg, N.J.). The forward primers used were: H-AP49, 5′-AAGCTTTAGTCCA-3′; H-AP50, 5′-AAGCTTTGAGACT-3′; H-AP51,5′-AAGCTTCGAAATG-3′; H-AP52, 5′-AAGCTTGACCTTT-3′; H-AP53, 5′-AAGCTTCCTCTAT-3′; H-AP54, 5′-AAGCTTTTGAGGT-3′; H-AP55, 5′-

AAGCTTACGTTAG-3′, and H-AP56, 5′-AAGCTTATGAAGG-3′, where H-AP refers to the primers supplied by the RNAimage™ kit. The RT-PCR products were size-fractionated in a 6% acrylamide gel and visualized by autoradiography.

Candidate suspensor-specific cDNAs as bands were identified that were (1) over 200 bp in size, (2) present at the same position in lanes containing cDNAs amplified from 6-8 DAP suspensor and micropylar-region mRNAs, and (3) absent in lanes containing cDNAs amplified from chalazal region, embryo proper, and leaf mRNAs. Isolated cDNA fragments were PCR-amplified, cloned into the pCR2.1® vector (Invitrogen: San Diego, CA), and sequenced. cDNAs were designated with (1) a C or G, indicating the anchor/reverse primer used, (2) a two-digit number between 49 and 56, indicating the forward primer used, and (3) a one-digit number indicating, the band position on the DD-RT-PCR gel. For example, C541 represents a cDNA band that was amplified by a C anchor/reverse primer, an H-AP54 forward primer, and that was in position number 1 on the DD-RT-PCR gel.

Gel blot analvsis of PCR-amplified population cDNAs

For pre-screening of differential display cDNA clones, PCR-amplifled cDNAs from different mRNA populations were generated following the procedures of Kelly et al. (1990), with minor modifications. Suspensor (6 DAP), ovule, 2 DAP seed, 3 DAP seed, 6 DAP micropylar region, 6 DAP chalazal region, and leaf total RNAs were isolated. First-strand cDNA was generated from 5 pg of each RNA using MMLV reverse transcriptase and 50 ng/μl of oligo(dT₂₀) as primer. The first-strand cDNAs were 3′ tailed with poly(dA) using terminal transferase. PCR amplifications were carried out using tailed first-strand cDNAs as templates and 2 μM of dT₂₀dN (where dN dG, dC, dA, or dT) as primer in 100 μl containing 20 mM Tris (pH 8.4), 50 mM KCl, 1 mM MgCl₂, and 0.2 μM dNTPs at 94° C./1 minute, 42° C./2 minutes, and 72° C./5 minutes for 30 cycles, followed by a 10 minute extension at 72° C. A 1 μl aliquot from each reaction was used to perform another round of amplification using the same conditions. The reactions were extracted with phenol/chloroform and precipitated in ethanol. An aliquot equivalent to 1 μg from each reaction was size-fractionated in a 1% agarose gel, which was then used for DNA gel blot analysis according to the procedures of Sambrook et al., supra.

DNA sequencing and analysis

DNA sequencing was performed following the dideoxy sequencing procedures recommended by USBiochemicals (Cleveland, Ohio). For genomic clone pG564g7.2.79, unidirectional, nested deletion set was prepared using the Erase-a-Base® system (Promega: Madison, Wis.). Compilation and analysis of sequences were carried out using the Wisconsin Genetics Computer Group (GCG) software. ORFs and exon-intron junctions were identified by using GENSCAN (http://ccr-081.mit.edu/GENSCAN.html; Burge, C., et al., Journal of Molecular Biology, 268:78-94 (1997)). The G564 intron-exon junctions were confirmed by comparing the cDNA and gene sequences. Protein sorting sequences were identified using PSORT (http://psort.nibb.ac jp; Nakai, K., et al., Genomics, 14:897-911 (1992)). DNA and protein sequence comparisons were performed using the NCBI Genbank BLAST programs (http://www.ncbi.nlm.nih.gov; Altschul, S. F., et al., Nucl. Acids Res., 25:3389-3402 (1997)). The complete C541 and 0564 cDNA sequences were based on sequences from (1) DD-RT-PCR cDNA clones, (2) cDNA clones isolated from a 5-9 DAP seed cDNA library, and (3) from cDNAs generated from 5′ random amplification of cDNA ends (RACE-RT-PCR; Chenchik, A., et al., Clontechniques, 10:5-8 (1995)).

In situ hybridization

In situ hybridization studies were carried out as described by Cox and Goldberg (Cox, K. H., et aL, PLANT MOLECULAR BIOLOGY: A PRACTICAL APPROACH (C. H. Shaw, ed. 1988) pp. 1-34) and Yadegari et al. (Yadegari, R., et al., Plant Cell, 6:1713-1729 (1994)) with minor modifications. Briefly, for Scarlet Runner Bean, unfertilized ovules and individual seeds (4-7 DAP) were harvested from pods, and seeds were cut at their chalazal ends before fixing to enhance penetration of the fixative. For tobacco, seeds up to 7 DAP were collected while still attached to the placenta. Older tobacco seeds were separated from the placenta prior to collection. Tissues were fixed overnight at 4° C. in 1% glutaraldehyde solution prepared in 0.1 M phosphate buffer (pH 7.0) (Meyerowitz, E. M., Plant Mol. Biol. Rep., 5:242-250 (1987)), dehydrated, cleared, and embedded in paraffin. Eight to 10 μm sections were hybridized to ³³P-labeled sense or anti-sense RNA probes at a specific activity of 4-5×10⁸ dpm/μg. After hybridization and emulsion development, sections were stained with 0.05% toluidine blue in 0.05% borate solution. Photographs were taken using either bright-field or dark-field illumination with a compound microscope (Olympus BH2: Olympus Corporation, Lake Success, N.Y.). The photographs were digitized, adjusted for optimum silver grain resolution using the KPT-Equilizer program (Metacreations Corp., Carpinteria, Calif.), and assembled in Adobe Photoshop 5.0 (Adobe Systems Inc., San Jose, Calif.).

Light microscopy

Bright-field microscopy

Seeds and unfertilized ovules from Scarlet Runner Bean were collected as described for in situ hybridization and fixed overnight in 5% glutaraldehyde, 0.1 M phosphate buffer (pH 7.0), and 0.01% Triton X-100 at 4° C. After dehydration, samples were embedded in Spurr's (Spurr, 1969) plastic resin (Polysciences: Warrington, Pa.). 1 μm thick sections were stained for 18 to 20 minutes at 42° C. with 0.05% toluidine blue in 0.05% borate solution. Bright-field photographs were taken with Kodak Gold 100 film (ISO 100/21°) using a compound microscope (Olympus BH-2: Olympus Corporation, Lake Success, N.Y.).

Whole mount microscopy

Dark-field photographs of seeds were taken using a dissecting microscope (Olympus SZH). Dark-field and bright-field photographs of dissected embryos were taken using a compound microscope (Olympus BH-2).

G564/GUS construction and tobacco plant transformation

A 21 kb G564 genomic clone was isolated from a Scarlet Runner Bean λDASHII (Stratagene: La Jolla, Calif.) genomic library by screening with a ³²P-labeled G564 cDNA clone. A 7 kb genomic fragment was recloned in pBluescript (Stratagene: La Jolla, Calif.) generating plasmid pG564g7.2.79. 4.8 kb of this plasmid was sequenced to confirm that the sequence of the coding region corresponded to that of the G564 cDNA clone. The entire G564g7.2.79 genomic clone was transferred into pGV1501AN, a pGV1500-derived plant transformation vector (DeBlaere, R., et al., Methods in Enzymology, 153:277-292 (1987)).

The region surrounding the ATG start codon in G564g7.2.79 was converted into an SphI endonuclease restriction site by PCR using a T3 primer and a mutagenic oligo (5′-ATTGGACTGCATGCTTACGCTAGTCTGTGCAGAG-3′). A 4.2 kb G564 promoter region was cloned in the SphI site upstream of the E coli β-Glucoronidase (GUS) gene coding region (Jefferson, R. A., et al., EMBO. J, 6(13):3901-3907 (1987)) in pGEM5GUS. After cloning, the G564 promoter region was re-sequenced. pGEMSGUS was constructed by inserting the GUS coding region and the Ti-plasmid gene 7 3′ end from TPI2/GUS gene (Drews, G. N., et aL, Plant Cell, 4:1383-1404 (1992)) into the NcoI/Notl sites of pGEM5 (Promega: Madison, Wis.). The G564/GUS gene was transferred to the pHYGA (Hygromycin^{R}) plant transformation vector (Klucher, K. M., et al., Plant Cell, 8:137-153 (1996)). Tobacco plants were transformed and regenerated using the leaf disk procedure of Horsch et al. (Horsch, et al., Science, 227:1229-1231 (1985)).

GUS histochemical assay

Transgenic tobacco seeds were harvested at different stages of development (Barker, S. J., et al., Proc. Natl Acad. Sci. USA, 85:458-462 (1988)). Embryos were dissected from seeds in 50 mM sodium phosphate (pH 7.0). Dissected embryos were incubated in GUS assay buffer [50 mM sodium phosphate (pH 7.0), 0.1% Triton X-100, 0.5 mM ferricyanide, 0.5 mM ferrocyanide, 2 mM 5-bromo-4chloro-3indolyl-βD-glucuronide] for 30 minutes to 16 hours at room temperature (Jefferson, R. A., et al., EMBO. J, 6(13):3901-3907 (1987)). Embryos were photographed under bright-field or dark-field illumination using a compound BH2 Olympus microscope.

### RESULTS

The Scarlet Runner Bean embrvo forms a “giant” suspensor early in development

The early developmental stages of Scarlet Runner Bean embryogenesis were characterized to link these stages to morphological markers of the developing seed and to specific times after pollination. Table 1 summarizes the morphological characteristics of the unfertilized ovule and developing seeds from 0 DAP until maturity at 35 DAP. From the ovule until 7 DAP, the seed length increased from 0.75 mm to 2-4 mm and the seed gradually adopted a green color (Table 1). At 11 DAP, the seed began to acquire red pigmentation in the area contiguous to the hilum region (Table 1) and the red color gradually spread and covered the entire seed by 20-25 DAP (Table 1). At 25 DAP, the seed length had increased and was 15 mm (Table 1). At 35 DAP, the mature dry seed had a purple seed coat with magenta streaks near the hilum and was 20 mm in length (Table 1).

The embryonic stages corresponding to seeds at different DAP were characterized from micrographs of longitudinal sections of the micropylar region containing the embryo. In the unfertilized ovule, the egg cell was identified from the orientation of its nucleus and cytoplasmic-dense region towards the chalaza and its vacuolated region towards the micropyle. These cytological features were inverted in the adjacent synergids. The egg cell and synergids were bordered by the central cell at their chalazal ends. At 2 DAP, the embryonic cells were irregularly organized, the apical and basal regions were morphologically indistinguishable, and endosperm had started to form. Just prior to globular stage (4 DAP), the suspensor of the filamentous embryo was distinguished from the embryo proper by its large and irregularly-shaped cells and was approximately 200-250 μm in length. By contrast, the embryo-proper cells were smaller and more uniform in size and shape.

The suspensor developed two distinct regions—a file of neck cells that connected suspensor to embryo proper and a set of large basal cells that protruded into the seed tissue. In the suspensor-basal region, the number of cells remained constant and the increase in length of the suspensor-basal region was mainly due to cell enlargement. The total suspensor length increased from 500 μm to 1000 μm, which was its maximum size (Table 1). The embryo proper increased in cell size and number, and developed from globular stage to heart stage, to cotyledon stage. At the cotyledon stage, the embryo proper was bigger than the suspensor and contained chlorophyll, whereas the suspensor remained white.

Globular embryos were dissected at the rate of approximately 10 per hour and collect separately the embryo-proper and suspensor regions (see Materials and Methods). Twenty micrograms of total RNA was isolated from 250 suspensors and 300 ng total RNA from 200 embryo-proper regions. Together, these data show that the suspensor of Scarlet Runner Bean embryo developed early in seed development (2-11 DAP) and that it was feasible to surgically dissect globular stage embryos into embryo-proper and suspensor regions in order to isolate region-specific embryo RNAs.

DD-RT-PCR of RNA from micro-dissected suspensor regions yields two suspensor-specific cDNA clones

Two strategies were used to identify suspensor-specific mRNAs (Materials and Methods): (1) differential screening of a 5-9 DAP seed cDNA library representing rnRNAs present in seeds containing globular-stage embryos and (2) DD-RT-PCR (Liang, P., et al., Science, 257:967-971 (1992)) of total RNA from micro-dissected suspensors of globular-stage embryos. Candidates for suspensor-specific cDNA clones were rescreened using: (1) DNA gel blots containing PCR-amplified population cDNAs (Materials and Methods) and (2) RNA gel blots (Materials and Methods).

Differential screening

In the first approach, two ‘seed-specific’ candidates for suspensor cDNA clones were identified, designated as SRB8 and SRB13, which hybridized with a 5-9 DAP micropylar-region seed cDNA probe, but not with a leaf cDNA probe (Materials and Methods). SRB8 and SRB13 were sequenced and used BLAST searches (Altschul, S. F., et al., Nucl. Acids Res., 25:3389-3402 (1997)) to show that the encoded proteins are homologous to ribosomal proteins and Bowman-Birk trypsin inhibitor, respectively (Materials and Methods).

DD-RT-PCR analysis

In the second approach, 25 candidate suspensor-specific cDNAs were identified that were displayed in the lane containing cDNAs amplified from 6 DAP suspensor RNA and in the lane containing cDNAs amplified from RNA of the micropylar half of 6 DAP seed, and that were not present in lanes containing cDNAs amplified from 6 DAP seed chalazal region RNA, globular-stage-embryo-proper RNA. and leaf RNA. All candidate cDNAs longer than 200 bp were cut from the gel, re-amplified, cloned, and sequenced (Materials and Methods).

Total cDNA gel blot analysis

Because the amount of RNA from the suspensor was too limited to screen a large number of clones by standard RNA blot analysis, a DNA gel blot procedure was devised using PCR-amplified population cDNAs (Kelly, A. J., et al., Plant Cell, 2:963-972 (1990)) to pre-screen the candidate cDNA clones (Materials and Methods). Total cDNA blot analysis of SRB8 and SRB13 showed that they hybridized with 6 DAP suspensor cDNA, unfertilized ovule, 2 DAP seed, 3 DAP seed, 6 DAP seed micropylar region cDNAs, and 6 DAP seed chalazal region cDNA but not with leaf cDNA. In addition, three DD-RT-PCR cDNAs were identified that hybridized with suspensor and seed micropylar-region cDNAs, but did not hybridize with ovule, seed chalazal-region, and leaf cDNAs. These three clones were designated as G541, G564, and G563, and represented putative suspensor-specific cDNAs. Sequence analysis and homology searches with these cDNAs indicated that they were not related to any protein of known finction. However, G564 and C541 proteins were predicted to be secreted or to be targeted to the vacuole, respectively (Materials and Methods).

RNA gel blot analvsis

SRB8, SRB13, G564, C541, and G563 probes were hybridized to gel blots, containing 6 DAP suspensor RNA, unfertilized ovule RNA, 2 DAP seed RNA, 3 DAP seed RNA, 6 DAP seed micropylar region RNA, 6 DAP seed chalazal region RNA, and leaf RNA to verify the results of the total cDNA blots. SRB8 and SRB13 probes hybridized with unfertilized ovule and all seed tissue RNAs, but not with leaf RNA. The SRB8 probe yielded a stronger hybridization signal with micropylar-region RNA than with chalazal-region RNA. By contrast, the SRB 13 probe produced a stronger signal with chalazal-region RNA as compared to micropyler-region RNA.

G564 and C541 probes did not hybridize with unfertilized ovule, 2 DAP seed, 3 DAP seed, 6 DAP chalazal region, and leaf RNAs. By contrast, G564 and C541 probes yielded a low signal with 6 DAP seed micropylar-region RNA. This signal was strongly amplified with suspensor RNA isolated from 6 DAP micropylar-region seed, suggesting that the lower signal with 6 DAP seed rnicropylar-region RNA was caused by dilution of the suspensor RNA by non-embryonic seed tissue RNA. G563 produced a similar hybridization pattern, but yielded equal hybridization signals with suspensor and 6 DAP micropylar RNAs. Together, these data showed that during seed development different patterns and levels of RNA accumulation occur. In addition, the higher hybridization signals from G564 and C541 probes with suspensor RNA versus micropylar RNA suggested that G564 and C541 cDNAs represent suspensor-specific mRNAs.

G564 and C541 are suspensor-specific markers

In situ hybridization was used to visualize directly regions that the G564, C541, G563, SRB8, and SRB13 mRNAs were localized in unfertilized ovules and 7 DAP seeds.

Localization of G564 and C541 mRNA

Dark field images of 7 DAP embryo sections hybridized with G564 and C541 anti-mRNA probes showed that G564 and C541 mRNAs were localized specifically in the suspensor. The G564 hybridization signal was spread evenly over the suspensor neck and basal cells. The C541 signal, on the other hand, was higher in the suspensor basal cells than in the suspensor neck cells. In addition, compared to the G564 probe, the C541 probe produced fewer hybridization grains, suggesting that the C541 mRNA is present at a lower prevalence than the G564 mRNA. No hybridization signal was detected above background level in the embryo proper, nor in any other cell or tissue of the developing seed. No G564 or C541 hybridization signals above background were observed in any unfertilized ovule cell or tissue type, similar to that observed with the sense control probe.

Localization of G563 mRNA

The G563 anti-mRNA probe hybridized specifically with transcripts in the endothelial layer surrounding the embryo but not in the embryo or any other seed tissue. The G563 hybridization signal was first detected at 3 DAP. By contrast, no hybridization signal above background level was obtained in the chalazal endotheium, nor in the endothelium or any other tissue of the unfertilized ovule.

Localization of SRBS and SRB13 mRNAs

The SRB8 and SRB13 mRNAs were highly prevalent within unfertilized ovule and seed, and were not localized exclusively within the suspensor. However, both mRNAs displayed different and changing accumulation patterns within pre- and post- fertilization ovule/seed. In the ovule, the SRB8 anti-mRNA probe detected transcripts in the endotheium and the epidermal layer. In addition, in the developing seed, SRBS hybridization grains accumulated to a high level in the endosperm and in the embryo. A stronger SRB8 hybridization signal was observed in the embryo proper than in the suspensor. The SRB13 anti-mRNA probe yielded hybridization signal in the outer integument of the unfertilized ovule and seed. Although SRB13 mRNA was present in the suspensor, its prevalence was not as high as in the integument.

Taken together, these data show that in the unfertilized ovule and developing seed various and partially overlapping transcript-accumulation patterns occur that change after fertilization has occurred. In addition, these results show that G563 mRNA is a marker for seed micropylar endothelium and that G564 and C541 mRNAs are suspensor-specific markers.

G564 and C541 are markers for the basal-region of the four-cell embrvo

In situ hybridization was used to investigate the accumulation pattern of G564 and C541 mRNAs during embryo development. Before fertilization, no hybridization signal was obtained with either G564 or C541 anti-mRNA probes in the egg or the synergids, even after a 6-9 month emulsion exposure. After fertilization, and before the suspensor and embryo-proper region were morphologically distinguishable (2 DAP), the G564 and C541 anti-mRNA probes detected transcripts exclusively in the two basal cells of the four-cell embryo, but did not detect any transcripts in the two apical cells. From early globular stage, after 3 DAP, G564 and C541 transcripts were detectable in the suspensor and not in the embryo proper. In addition, the higher concentration of C541 mRNA in the suspensor-basal region, compared with the suspensor-neck region.

The G564 mRNA accumulation pattern at later stages of embryo development was investigated in 23 DAP early-maturation-stage embryos. The dark field image of an axis and cotyledon section that was hybridized with a G564 anti-mRNA probe showed that G564 transcripts accumulated in the axis, but not in the cotyledons or in any other seed tissue.

Together, these data show that late G564 transcripts mark the embryo axis, and that G564 and C541 mRNAs are suspensor-specific markers. In addition, these results show that within two cell divisions after fertilization, G564 and C541 mRNAs mark the two basal cells of the four-cell embryo.

Basal-region specific G564 mRNA accumulation is transcriptionally regulated

The G564 gene was isolated from a Scarlet Runner Bean genomic library to determine whether the basal-region-specific and suspensor-specific G564 mRNA accumulation pattern was regulated at the transcriptional or post-transcriptional levels. A 6.99 kb genomic fragment from the Scarlet Runner Bean was isolated. The G564 coding region was 659 bp long, consisted of 2 exons of 107 and 388 bp, and contained one 164 bp intron. The 5′ and 3′ regions, included in the genomic fragment, were 4242 bp and 2085 bp in length respectively. In the 5′ region, another gene, at position -4214 to -2588, similar to the Arabidopsis Pol3 gene (accession no. AC005561) was identified.

G564 mRNA localization in transgenic tobacco embryos carrying the Scarlet Runner Bean G564 gene

The Scarlet Runner Bean G564 genomic clone was introduced into tobacco and localized G564 mRNA accumulation in transgenic embryos to investigate whether the basal-region-specific and suspensor-specific G564 mRNA accumulation patterns were conserved in a heterologous plant. At the pie-globular embryo stage, similar to the Scarlet Runner Bean embryo, the G564 mRNA accumulated specifically in the embryo basal region, but not in the apical region. At this stage of tobacco embryo development the suspensor is distinguishable from the embryo proper. At the globular stage, the G564 mRNA was detected in the suspensor and in the hypophyseal region of the embryo proper. In heart- and torpedo-stage embryos, G564 transcripts accumulated in the axis similar to the G564 mRNA accumulation pattern in the Scarlet Runner Bean early maturation-stage embryo. In addition, G564 transcripts accumulated in the endosperm. No hybridization signal above background level was detected in non-transformed tobacco embryos. Together, these results suggested that the basal-region-specific and suspensor-specific G564 mRNA accumulation pattern is conserved across the plant kingdom and that all regulatory elements for correct suspensor-specific G564 mRNA accumulation are contained within the 6.99 kb G564 genomic clone. Analysis of the gene sequence indicated that the coding sequence was interrupted by an intron. As measured from the first identified nucleotide of the G654 cDNA sequence (i.e., position 4242 of SEQ ID NO:2), the first exon is located from positions 1 to 107 and the second exon from positions 271-659.

G564/GUS expression in transgenic tobacco embrvos

A chimeric G564-promoter/GUS gene was introduced (see Materials and Methods) into tobacco and accumulation of GUS mRNA and GUS enzyme activity in transgenic tobacco embryos was monitored to study G564 transcription regulation. The G564/GUS gene was active in the two suspensor cells of the five-cell pre-globular embryo. In the embryo proper, by contrast, no GUS activity was detected. No GUS hybridization grains were detected above background level, indicating that—in the suspensor—GUS mRNA had accumulated below the detection level of the in situ hybridization. At globular stage, both GUS activity and GUS mRNA accumulation were detectable in the suspensor and in the hypophyseal region of the embryo proper. At heart and torpedo stages, GUS activity and mRNA accumulation were detectable in the axis. GUS transcripts were also detected in the endosperm. Together, these data show that in transgenic tobacco embryos, G564/GUS expression and GUS mRNA accumulation follow the same developmental pattern as was observed for G564 transcripts in transgenic tobacco embryos carrying the entire G564 gene and as observed in Scarlet Runner Bean embryos. In addition, these results indicate that the G564 mRNA basal-region-specific and suspensor-specific accumulation is controlled at the transcriptional level by the 4.2 kb 5′ upstream region of the G564 gene, and that the transcription-regulatory finction of this region was conserved between plant species.

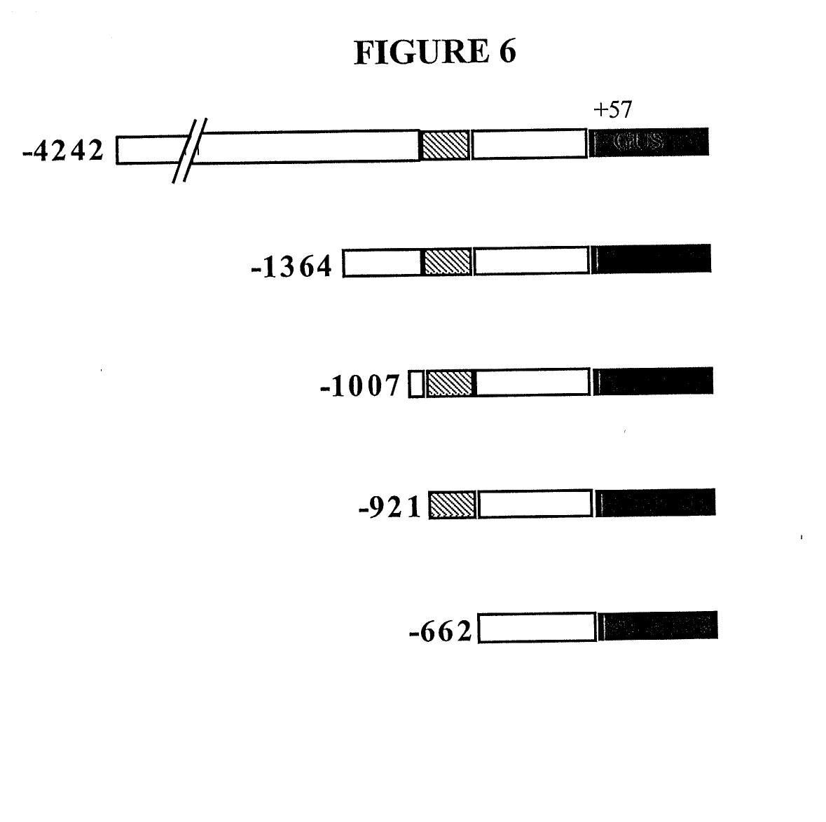

To further analyze the G564 promoter, a series of 5′ deletions were constructed and tested for suspensor-specific activity (FIG. 6). Promoters with deletions of nucleotides -4242 to -921 retained suspensor-specific GUS activity, while promoters with deletions up to nucleotide -662 did not have GUS activity in suspensor cells. These results indicate that a suspensor-specific control element is present between positions -921 and -662.

Sequence analysis of the Scarlet Runner Bean G564 promoter region revealed four sequences of approximately 100 base pairs long within the promoter region. Each repeat is highly homologous to the other repeats. These repeats can be found between positions -1327 to -1225, -1206 to -1103, -1030 to -928, and -908 to -800. Further analysis reveals that 80 base pair subsequences within the 100 base pair sequences are particularly conserved (- 1327 to -1247, -1183 to -1105, -1030 to -950 and -885 to -805. Each homologous repeat contains either the sequence GAAAAGCGAA (SEQ ID NO:10) or the related sequence GAAAAGTGAA (SEQ ID NO:l l). Further functional analysis demonstrated that -1368 to - 1208 of the G5564 promoter containing one of the 80 base pair sequences described above, was sufficient to drive suspensor-specific GUS expression from a minimal CaMV 35S promoter.

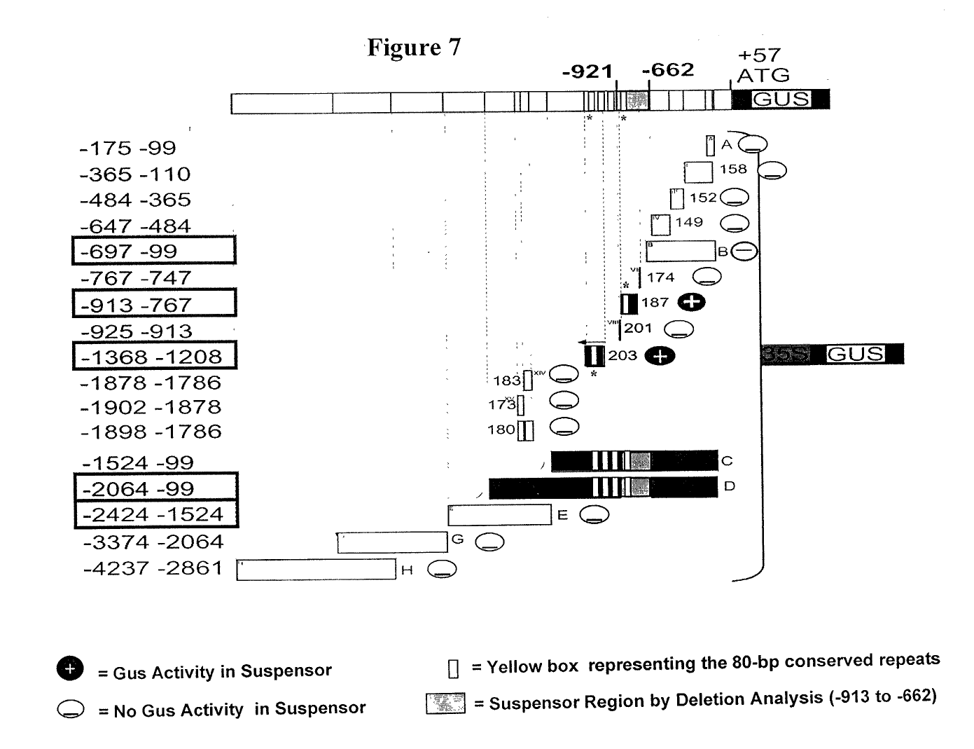

Additional promoter fragments from the Scarlet Runner Bean G564 promoter were isolated and linked to a minimal 35S promoter operably linked to the GUS gene. As indicated in FIG. 7, two fragments encompassing the region between -921 and 662 resulted in GUS activity in the suspensor cell. These fragments were from positions -1524 through -99 and -2064 through -99. In addition, a 187 base pair fragment (positions -913 through -713 of FIG. 1) linked to the minimal 35S promoter lead to GUS expression in the suspensor cell. This result suggests that at least one suspensor-specific control element is located within the 187 base pair fragment.

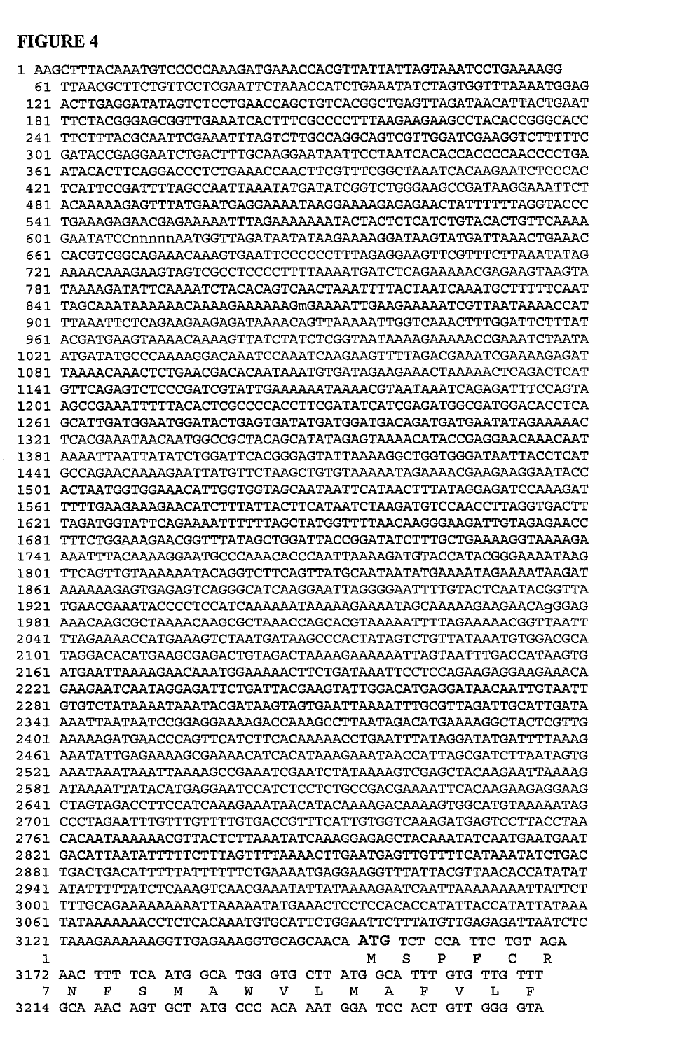

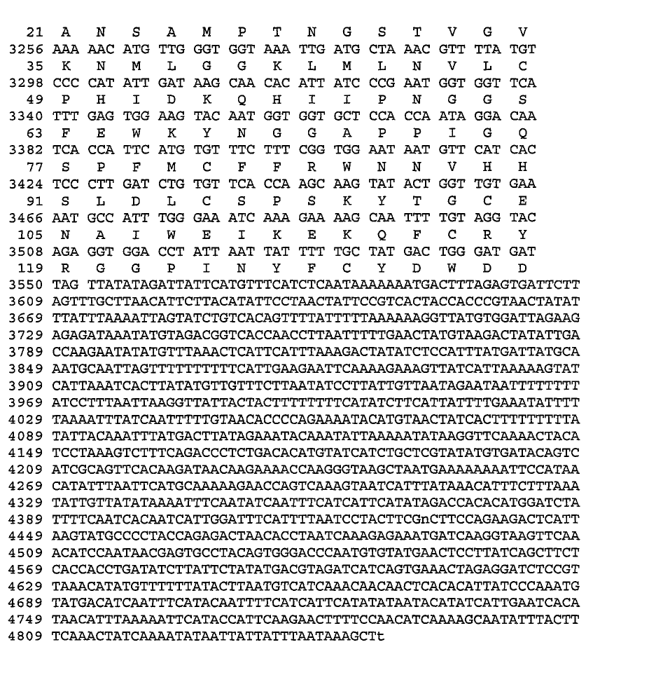

A comparison of the Scarlet Runner Bean G564 promoter (SEQ ID NO: 1) and the Scarlet Runner Bean C541 promoter identified a conserved 10 base pair sequence which may confer suspensor-specific activity. Supporting this assertion, the sequence, GAAAAGCGAA (SEQ ID NO:10), is found at positions -846 to -837, i.e., within the area which the deletion results indicate controls suspensor-specific activity. Identical motifs can also be found at positions -1144 through -1135 and between -713 through -704 of FIG. 1. The motif is also found at positions -684 through -675 of the Scarlet Runner Bean C541 promoter region (FIG. 4). Interestingly, the Arabidopsis G564 ortholog promoter region comprises a motif (GAAAAGCCAA - SEQ ID NO:12) that is highly homologous to SEQ ID NO: 10.

As a further analysis, a series of embryo-specific promoters that do not initiate transcription in the suspensor cell were screened for SEQ ID NO: 10. None of the promoters screened (Kti1 (Accession No. 45035), Kti2 (Accession No. S45035), Kti3 (Accession No. K00821) or the lectin promoter (Accession No. S45092)) contained SEQ ID NO: 10.

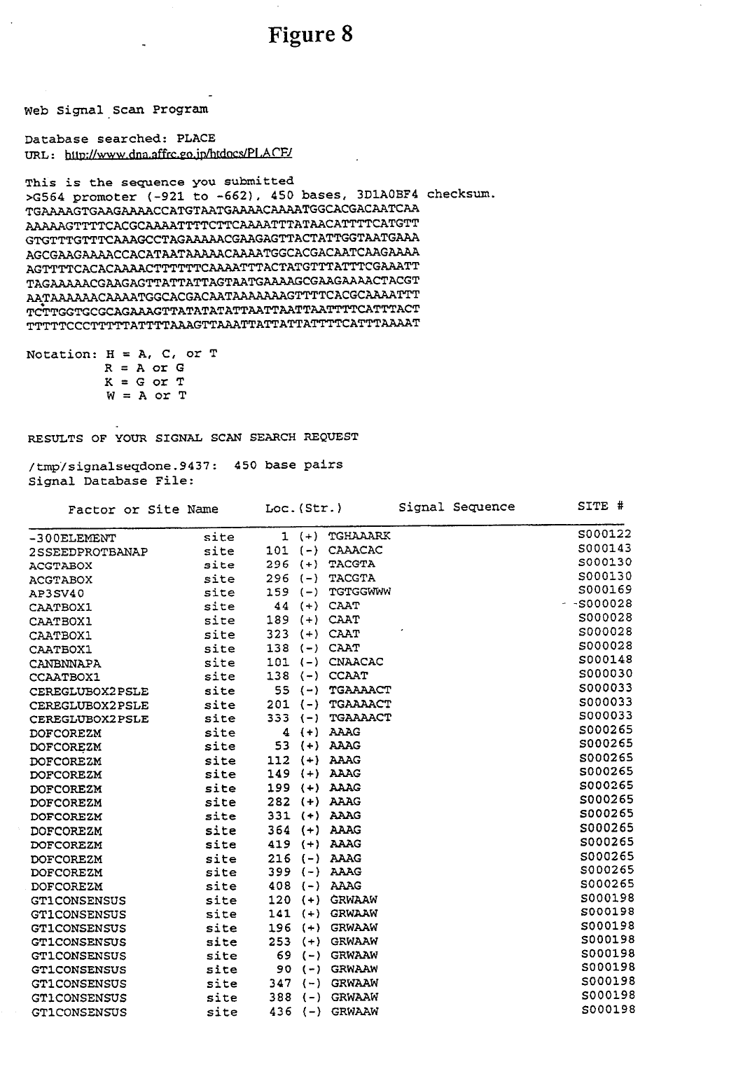

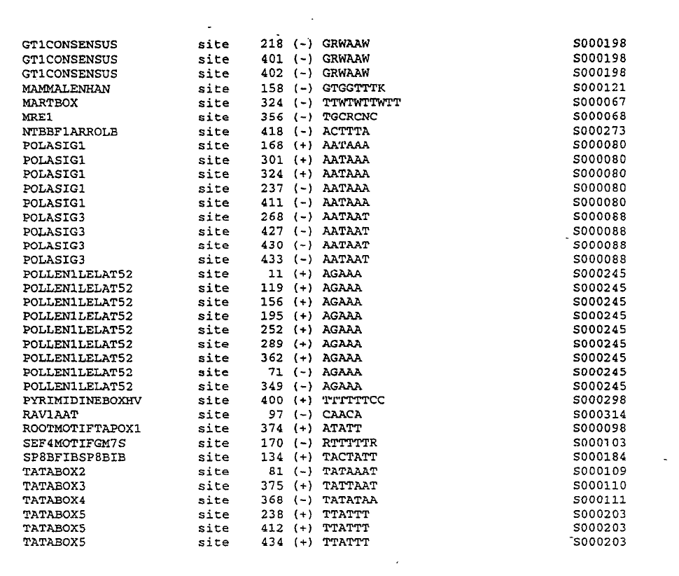

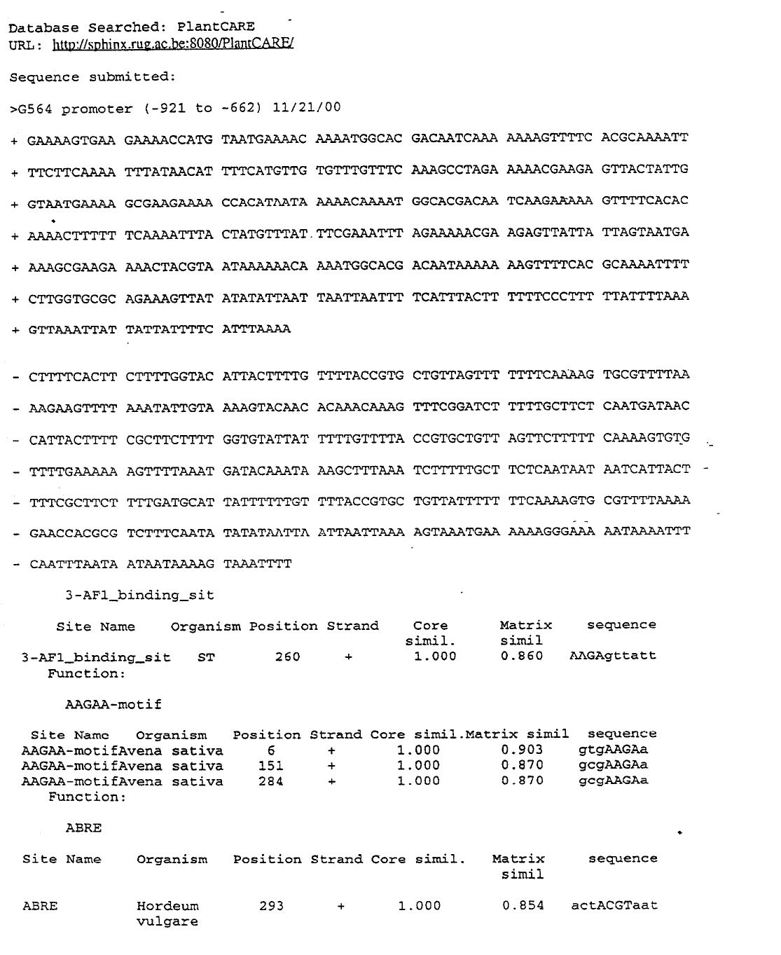

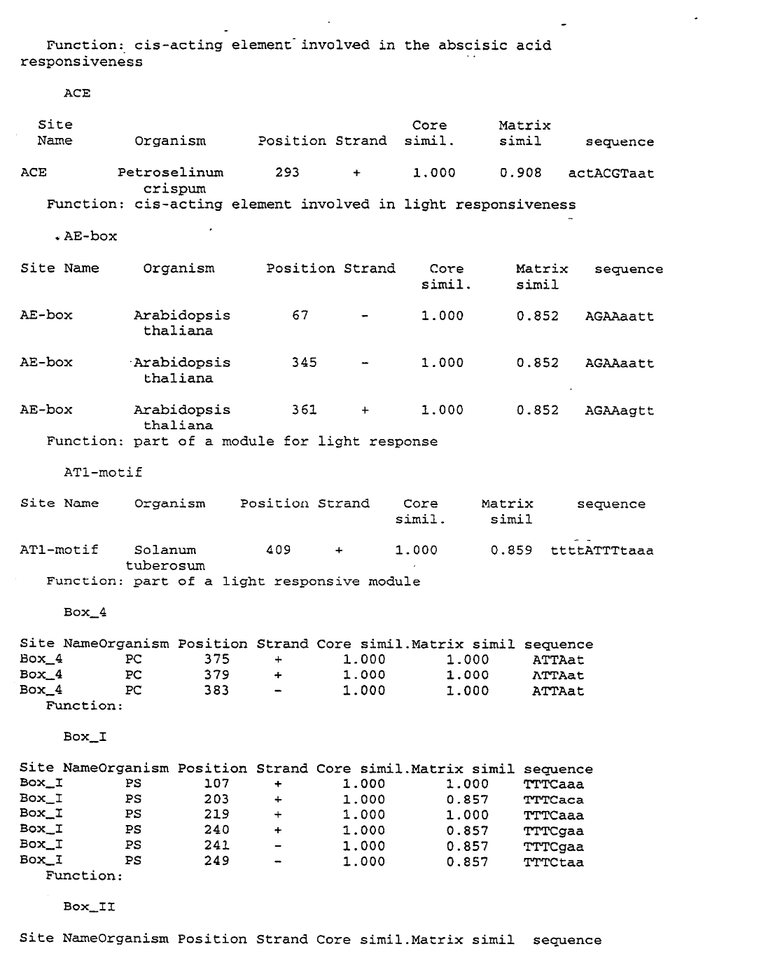

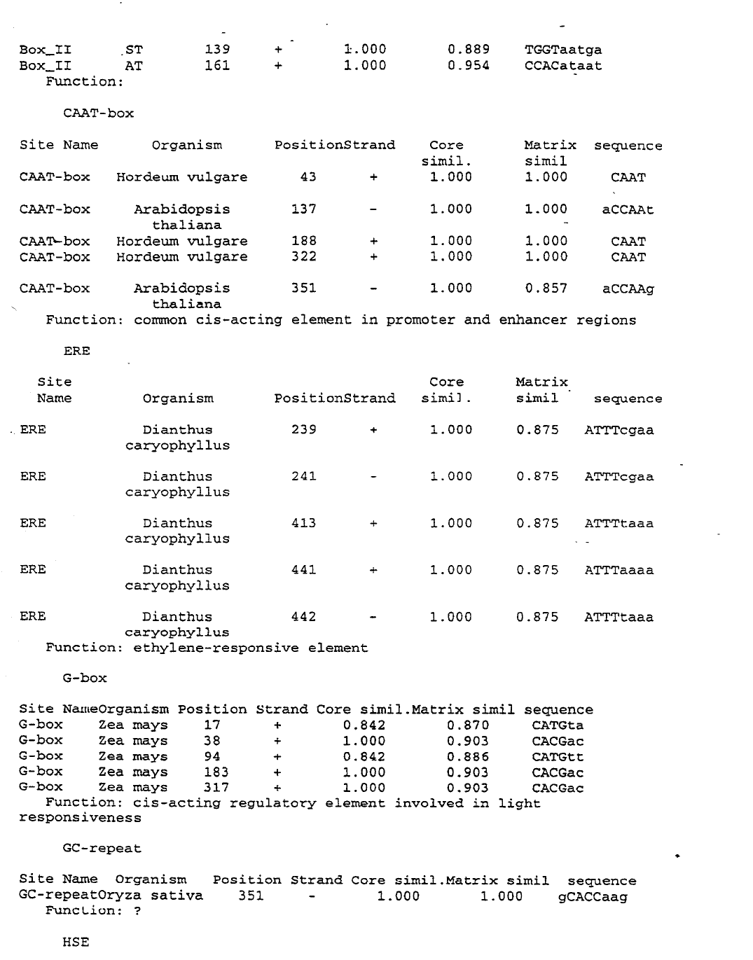

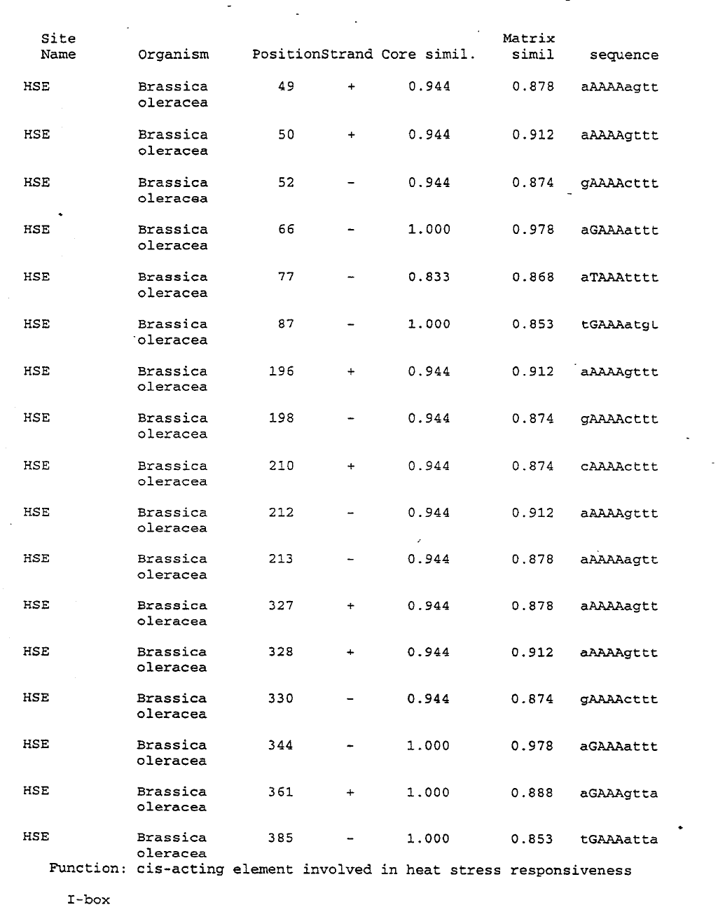

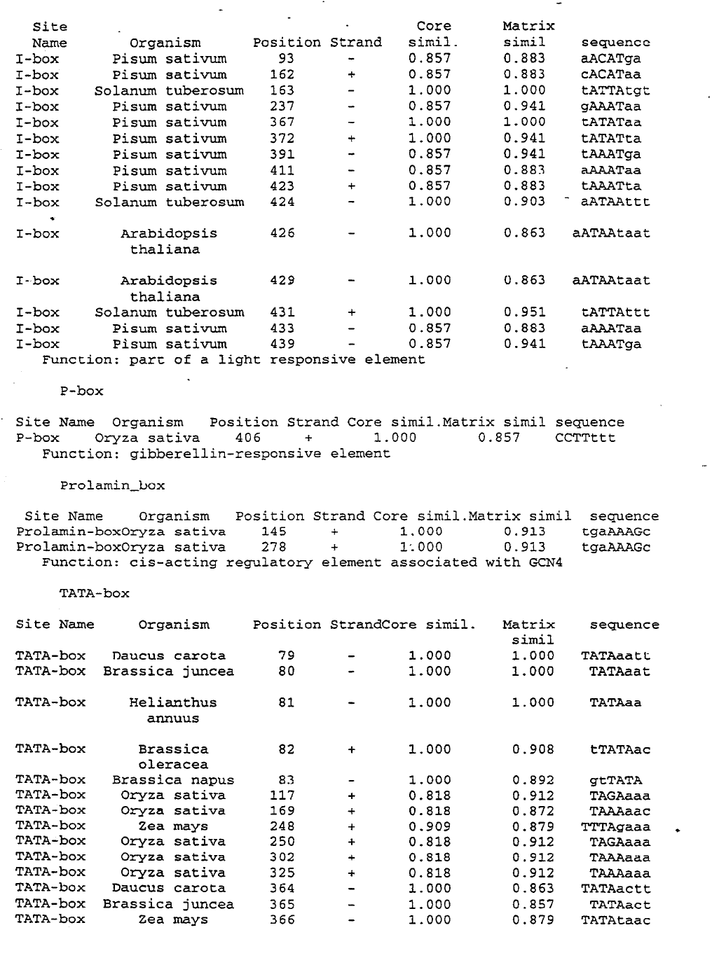

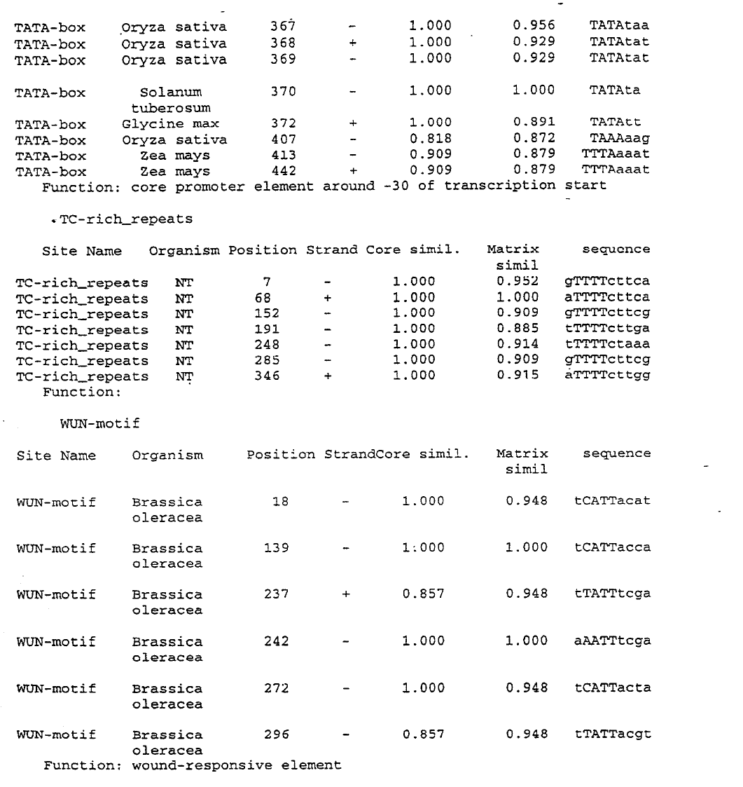

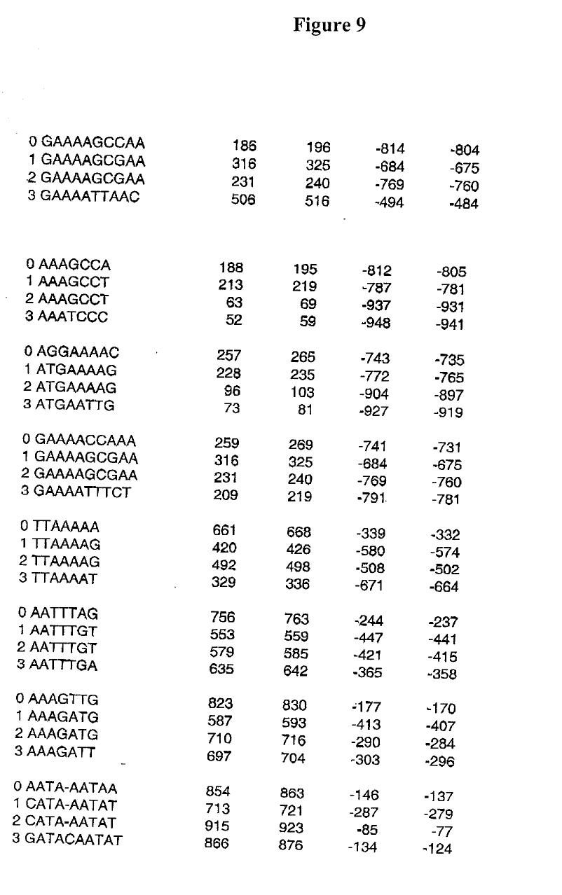

A listing of other motifs identified in the region defined by -921 to -662 of the Scarlet Runner Bean G564 promoter region is provided as FIG. 8.

### DISCUSSION

The Scarlet Runner Bean embryo was used as a model system to investigate gene expression programs during early embryogenesis. Two suspensor-specific mRNAs designated as G564 and C541 were identified. In four-cell embryos, G564 and C541 mRNAs accumulate exclusively in the two basal cells, but are not detectable in the two apical cells. A chimeric G564/GUS reporter gene is transcribed specifically in two basal cells of transgenic tobacco embryos at a similar stage (five-cell). From these results it is concluded that as early as the four-cell embryo stage the apical and basal cells transcribe different gene sets and are specified at the molecular level.

The Scarlet Runner Bean suspensor is a novel system to studv the mechanisms regulating specification of the basal region of the early plant embrvo

Scarlet Runner Bean has been used historically to study the role of the suspensor in embryo development. The suspensor size facilitated its micro-dissection (FIG. 1O-Q) and rendered it accessible for physiological and cytological studies (Nagl, W., Z. Pflanzenphysiol., 73:1-44 (1974): Sussex, I., et al., Caryologia, 25:261-272 (1973); Yeung, E. C., et al., Protoplasma, 94:19-40 (1978); Yeung, E. C., et al., Plant Cell, 5:1371-1381 (1993); Yeung, E. C., et al., Zeitschrift fur Pflanzenphysiology, 91:423-433 (1979)). Because the suspensor is simple, terminally differentiated, and only few cell generations removed from the basal cell, we have adopted this model to study the mechanisms specifying basal-cell fate. Scarlet Runner Bean suspensors were collected separately from embryo propers and used the suspensors to identify two genes, G564 and C541, that are transcribed specifically in the suspensor and in the basal region of the embryo shortly after division of the zygote. The G564 promoter maintains transcriptional activity in suspensors of tobacco embryos. Therefore, this promoter can be used to identify regulatory genes and thus as an entry point to penetrate the regulatory circuits that control basal cell specification. In addition, Arabidopsis genes corresponding to G564 and C541 were identified (SEQ ID NO:4 and SEQ ID NO:8, respectively). We can use these genes to find mutants important for suspensor function in embryo development. Thus, the Arabidopsis model system is complemented by the Scarlet Runner Bean suspensor as a model to investigate the earliest events in plant embryogenesis.

A mosaic of gene expression programs is active during seed development

In flowering plants, fusion of the sperm cells with both the egg cell and central cell initiates embryo and endosperm development, respectively (Table 1). In addition, fertilization causes the integument and the endothelium to differentiate and to contribute to the development of the seed (Table 1 and embryology of angiosperms (Johri, B. M., ed. 1984); Miller, S. S., et al., Annals ofBotany London, 84:297-304 (1999); embryogenesis in angiosperms: a developmental and experimental study (Raghavan, V., ed. 1986)). Simultaneously, a cascade of different gene expression programs is initiated that are correlated with the various events occurring during embryo and seed development (Goldberg, R. B., et al., Cell, 56:149-60 (1989); Goldberg, R. B., et al., Science, 266:605-614 (1994)). For example, SRB8 mRNA accumulates in the ovule chalazal endothelium and after fertilization, it accumulates in endosperm and embryo proper. SRB8 is homologous to a ribosomal protein L10A indicating a greater need for ribosome and protein synthesis in these tissues before and during early seed development SRB 13 transcripts accumulate in the integuments and, after fertilization, in the seed coat and to a lesser extent in the developing embryo. SRB13 is homologous to a Bowman-Birk trypsin inhibitor illustrating the protective function of integuments and seed coat.

G563 mRNA starts to accumulate specifically at 3 DAP in the seed micropylar endothelium surrounding the developing embryo. The micropylar-endotheium cell layer is suggested to function as an embryo-nursing tissue by exchanging metabolites with the suspensor via extensive cell wall ingrowths that appear at 3 DAP (Natesh, S., et aL, Embryology of angiosperms, (ed. B. M. Johri) pp. 377-444, Berlin: Springer Verlag (1984); Yeung, E. C., et al., Protoplasma, 94:19-40 (1978); Yeung, E. C., et al., Can. J Bot., 57:120-136 (1979)). Probably because of this tight contact between endothelium and suspensor, some residual endotheial cells were present in our hand-dissected suspensor preparations, which explains why we were able to identify G563 as a micropylar-endothelium-specific transcript. The correlation of G563 transcript accumulation with the appearance of cell wall ingrowths contiguous to the suspensor of the developing embryo suggests that G563 marks the specification of the micropylar endotheium as an embryo-nursing tissue. Although the function of the predicted G563 protein is unknown, its high glycine and praline content (47.5 and 12.5 percent, respectively) suggests a structural finction perhaps in the formation of the specialized cell wall ingrowths.

G564 and C541 transcripts accumulate specifically in the suspensor. G564 transcripts are distributed evenly over the whole suspensor, while C541 transcripts accumulate to a higher concentration in the suspensor-basal region than in the suspensor-neck region. Based on physiological and cytological studies, the main activities of the suspensor are importing, producing and transporting nutrients and growth regulators to the developing embryo proper (Alpi, A., et al., Planta, 147:225-228 (1979); Brady, T., Cell Diferentiation, 2:65-75 (1973); Ceccarelli, N., et al., Zeitschrift fur Pflanzenphysiology, 102:37-44 (1981); Clutter, M., et al., Journal of Cell Biology, 63:1097-1102 (1974); Schnepf, E., et al., Protoplasma, 69:133-143 (1970); Sussex, I., et al., Caryologia, 25:261-272 (1973); Yeung, E. C., et al., Can. J Bot., 57:120-136 (1979); Yeung, E. C., et al., Plant Cell, 5:1371-1381 (1993)). The exact functions of G564 and C541 in these activities are unknown, but the fact that G564 protein is predicted to be secreted suggests that it might play a role in metabolite exchange in the intercellular space of the cell wall ingrowths. C541 is predicted to be targeted to the vacuole, which explains the higher concentration of C541 mRNA in the highly vacuolate suspensor-basal region.

Together, the different SRB8, SRB13, G563, G564, and C541 mRNA accumulation patterns illustrate that an array of different gene regulatory programs is active to make a seed. However, how these programs are regulated coordinately remains to be established.

Differentiation of early-embrvo apical and basal regions is marked by the accumulation of different transcript sets

The suspensor is derived from the basal cell of the two-cell embryo, however it is not known what mechanisms direct the basal cell to become specified and develop into a suspensor, nor is it known when these mechanisms are active. To gain entry into the mechanisms regulating suspensor development and thus into the mechanisms regulating apical-basal cell specification events, two suspensor-specific transcripts were identified, designated as G564 and C541. The G564 and C541 transcripts first accumulate in the two basal cells of the four-cell embryo, before the suspensor is morphologically distinguishable and thus marking the embryo-basal region for suspensor specification. By contrast, in Arabidopsis pro-embryos a homeobox mRNA, designated as ATML1, has been found to accumulate selectively in the apical cell (Lu et al., Plant Cell 8(12):2155-68 (1996). Together, this shows that at the four-cell embryo stage the apical and basal regions have differentiated and that this specification process is marked by accumulation of different transcript sets. In addition, it indicates that the mechanisms activating the apical and basal- region-specification processes are active earlier either in he two-cell embryo or in the zygote or egg.

Apical and basal-region specific accumulation of mRNA is caused by specific transcriptional programs

G564 mRNA accumulation pattern in the basal-region and the suspensor is similar to that in Scarlet Runner Bean embryos. This shows that the 6.99 kb G564 genomic clone is a marker for the specification mechanism of the basal region of the four-cell embryo and that within this 6.99 kb genomic fragment an elements are present that are recognized by this mechanism. In addition, we conclude that although early-embryo cell division patterns are different between Scarlet Runner Bean and tobacco (Kaplan, D. R., et aL, Plant Cell, 9:1903-1919 (1997); Natesh, S., et al., embryology of angiosperms, (B. M. Johri, ed. 1984) 377-444), the mechanisms specifying cell fate are conserved (Goldberg, R. B., et al., Science, 266:605-614 (1994)).

In transgenic tobacco embryos containing the chimeric G564/GUS gene, GUS enzyme activity in a basal-region-specific and suspensor-specific pattern are similar to the G564 mRNA accumulation pattern in Scarlet Runner Bean embryos and G564 transgenic tobacco embryos. This shows that the mechanism regulating basal-region specific G564 mRNA accumulation works at the transcriptional level. Therefore, the differentiation of the basal and the apical regions of the early embryo, which is marked by differential accumulation of transcript sets, is caused by specific apical and basal-region transcription programs. Initial analysis was performed of the basal-region transcription program by dissecting the GYM promoter for cis-regulatory elements to identify its regulatory factors. Preliminary data indicate that the elements directing basal-region-specific transcription are present at -921 to -662.

A model for the mechanism of specification fo the apical and basal cell of the two-cell embryo

How is the G564 transcriptional program activated specifically in the embryo basal region and how does this provide clues to the general mechanism specifying basal-cell fate? A possible explanation might reside in the apical-basal polarized cyto-architecture of the egg cell and zygote (FIG. 1E and Willemse, M. T. M., et al., embryogeny of angiosperms, (B. M. Johri, ed. 1984) 159-196). The asymmetric distribution of cytoplasm, and/or its contents within the egg and/or zygote may play a role in activating specific apical and basal-region transcription programs (Goldberg, R. B., et al., Science, 266:605-614 (1994)). Based on this suggestion, a simple model is proposed for the specification of basal cells leading to suspensor differentiation. This model assumes that there is an asymmetric distribution of “morphogenetic factors” (e.g. transcription factors) within either the egg cell or the zygote or both. In addition, it assumes that the basal cell (and suspensor) is specified autonomously as a consequence of inheriting the ‘morphogenetic factors’ following zygotic division. These factors trigger a cascade of events leading to the transcription of basal- region-specific genes, like G564, and suspensor differentiation (FIG. 8).

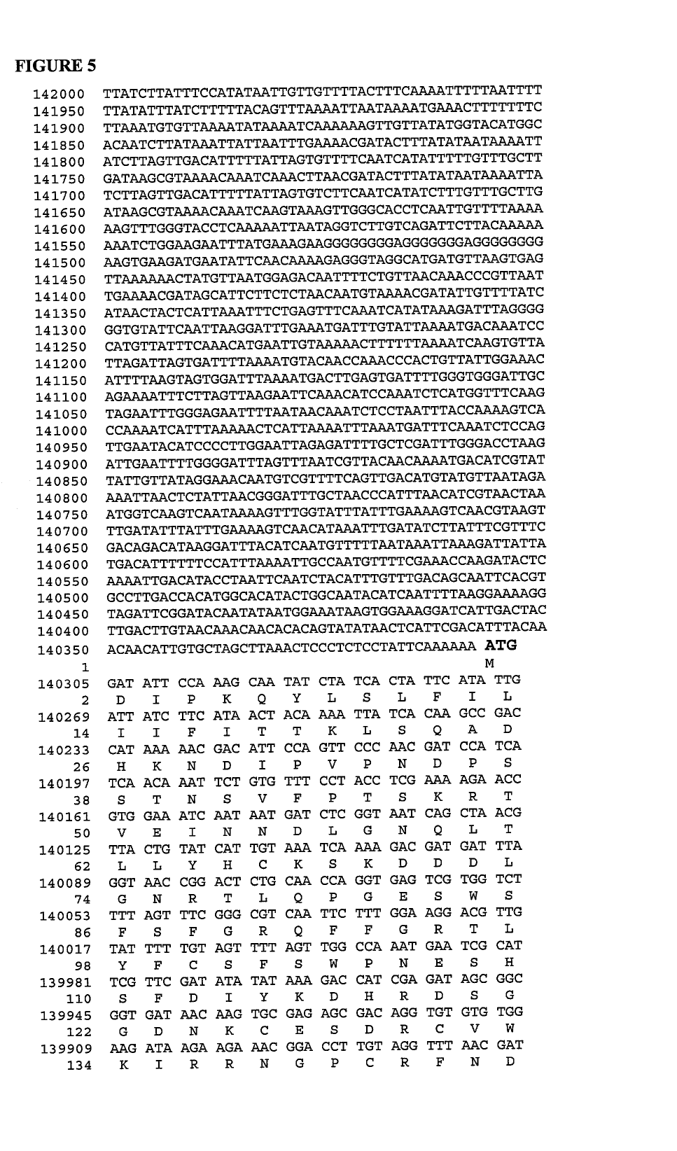

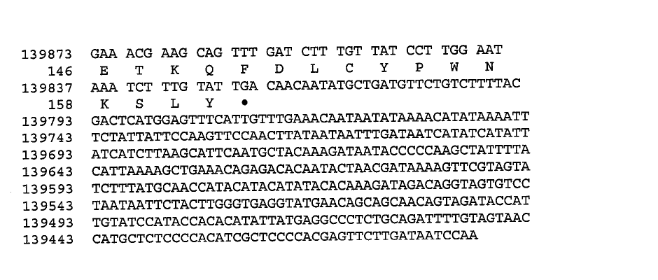

The model outlined above is consistent with analogous autonomous specification processes that occur for specific cell types during embryo development in various animal systems (Davidson, E. H., et al., Development, 125:3269-3290 (1998)). In plants, this model predicts that the embryo-basal-region-specific transcription of G564 (FIG. 5B, 7B, J) is programmed by one or more basal-cell-specific transcription factors, and that these transcription factors are derived initially from the basal region of the egg cell or zygote. It is possible that these regulatory factors are bound by the cytoskeleton to the basal pole of the egg and/or the zygote and that these factors automatically become pan of the basal cell after zygote division. This would be similar to the mechanism responsible for targeting factors to unique intracellular cytoplasmic locations in animal embryos (Lall, S., et al., Cell, 98:171-180 (1999); Yisreali, J. K., et al., Development, 108:289-298 (1990)) and to the mechanism by which the polarized axis is fixed in Fucus eggs (Kropf, D. L., Plant Cell, 9:1011-1020 (1997); Quatrano, R., Cold Spring Harbor Symposia on Quantitative Biology, 57:65-70 (1997)).

Alternatively, it is also possible that a signalling mechanism is responsible for basal cell specification similar to that which establishes dorsal/ventral polarity in Drosophila embryos (Davidson, E. H., et al., Development, 125:3269-3290 (1998); Sen, J., et al., Cell, 95:471-481 (1998)). In this case, a signal derived from the maternal seed tissues contiguous with the basal cell (e.g. endotheium) would interact with a basal cell ligand which would then trigger a signal transduction cascade leading to transcription of basal-region-specific genes like G564 and suspensor differentiation. One prediction of this model is that the transcription factors which activate G564 transcription should be present in both the apical and basal cells of the embryo, but remain inactive within the apical cell (Davidson, E. H., et al., Development, 125:3269-3290 (1998)).

TABLE 1
Description of Scarlet Runner Bean seed development stages.

| | DAPs after | | | |
|---|---|---|---|---|
| | Pollination | | | |
| Stage | (DAP) | Suspensor length | Seed length | Seed color |
| Ovule | 0 | — | <0.75 mm | white |
| Proembryo | 1 to 4 | <50 μm to 250 μm | 0.75 to 1.5 mm | pale green |
| Globular | 5 to 9 | 320 μm to 600 μm | 2 to 4 mm | green |
| Heart | 10 to 12 | 700 μm to 900 μm | 4.5 to 6 mm | green with red |
| | | | | pigment contiguous |
| | | | | to the hilum |
| Early cotyledon | 13 to 17 | ˜1000 μm | 7 to 9 mm | green with heavy red |
| | | | | pigment in the area |
| | | | | surrounding the |
| | | | | hilum |
| Late cotyledon | ˜25 | ND | ˜15 mm | scarlet red |
| Mature | ˜30 to 35 | ND | ˜20 mm | purple |

ND, not determined

It is understood that the example and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. An expression cassette comprising a promoter sequence comprising SEQ ID NO:10, SEQ ID NO:11 or SEQ ID NO:12 and a promoter polynucleotide with at least basal promoter activity, which promoter sequence is operably linked to a heterologous polynucleotide, wherein when the expression cassette is inserted into a plant, the heterologous polynucleotide is specifically expressed in a suspensor cell and/or basal region of a plant embryo.

2. The expression cassette of claim 1, wherein the promoter sequence comprises SEQ ID NO:10.

3. The expression cassette of claim 1, wherein the promoter sequence comprises SEQ ID NO:11.

4. The expression cassette of claim 1, wherein the promoter sequence comprises SEQ ID NO:12.

5. The expression cassette of claim 1, wherein the promoter is operably linked to the heterologous polynucleotide in an antisense orientation.

6. The expression cassette of claim 1, wherein the promoter is operably linked to the heterologous polynucleotide in a sense orientation.

7. A vector comprising an expression cassette of claim 1.

8. A host cell comprising the vector of claim 7.

9. A transgenic plant comprising the expression cassette of claim 1.

10. A method of constructing a promoter that specifically induces transcription in a plant suspensor cell and/or basal region of a plant embryo, the method comprising (i) providing a promoter polynucleotide capable of at least basal promoter activity in a plant; (ii) inserting a nucleic acid comprising SEQ ID NO:10, SEQ ID NO:11 or SEQ ID NO:12 within or adjoining the promoter polynucleotide, thereby constructing a test promoter; and (iii) assaying the test promoter to determine whether the test promoter specifically initiates transcription in a suspensor cell and/or basal region of a plant embryo.

11. The method of claim 10, wherein the nucleic acid comprises SEQ ID NO:10.

12. The method of claim 10, wherein the nucleic acid comprises SEQ ID NO:11.

13. The method of claim 10, wherein the nucleic acid comprises SEQ ID NO:12.

14. A method of modulating transcription in a plant suspensor cell and/or basal region of a plant embryo, the method comprising introducing into a plant an expression cassette of claim 1.

15. The method of claim 14, wherein the nucleic acid comprises SEQ ID NO:10.

16. The method of claim 14, wherein the nucleic acid comprises SEQ ID NO:11.

17. The method of claim 14, wherein the nucleic acid comprises SEQ ID NO:12.

18. The method of claim 14, wherein the promoter is operably linked to the heterologous polynucleotide in an antisense orientation.

19. The method of claim 14, wherein the promoter is operably linked to the heterologous polynucleotide in a sense orientation.


SEQUENCE LISTING (42 sequences)

SEQ ID NO: 1 - DNA, 4298 bp, Phaseolus coccineus
  promoter (1)..(4298): Scarlet Runner Bean G654 promoter
gcatgcactgccacaagtagtgaactcatggttttacctcctcaagtagaaaaccttttgagtgaatttgaagatttattctcccaagaaggacccattgggcttcctcctcttagggggatagaacatcaaattgactttataccgggggcaagcctaccaaataggcctccttatagaaccaaccccgaggaaacaaaggagatagaatcacaagttcaagacttgttggagaagggttgggttcaaaagagcctaagcccttgtgctgtacctgtcttgttggtgccaaaaaaagatggaaaatggcgtatgtgttgtgattgtagagcaatcaacaacatcaccatcaagtataggcatccaatcccaaggcttgacgatatgcttgatgaattgcatgggtcaactctattctccaaaattgaccttaaaagtggatatcaccaaattcgaatcaaggagggtgatgagtggaaaaccgcttttaagaccaaatttggattatatgagtggttggtgatgccctttggtcttactaacgctccaagtacattcatgaggcttatgaatcacaccttgagggattgtataggtaaatatgtagtagtttattttgatgatatcttagtatatagtaaaaccctagaagaccatctaagtcaccttagggaagttcttctagttcttaggaaaaatagtctttttgccaatagggataagtgtaccttttgtgtagatagcgtagtctttttaggctttatagtaaaccaaaagggggtgcatgtagatcccgagaaaatcaaagccatccgcgagtggccaactccacaaaatgtaagtgatgtgagaagttttcatgggttagctagcttctatagaaggtttgttcccaatttttctagcctagcttctcccttgaatgaacttgtaaaaaaagatgttgcattttgttggaatgaaaagcatgagcaagcctttcaaaggctaaaagctcactcaccaatgcacccatcctatctcttccaaatttttccaaacttttggagatagagtgtgatgcatcgggagtaggcatagtgcggttttgttgcaaggtggacaccccttgcttattttagtgaaaaactccatggtgccaccctcactaccccacctatgacaaagactctatgctcttgtgcgaccctaaagacttggggaacactaccttgngtcccaaagaatttggntatccatagtgatcacgagtctttaaaatatttaaagggccaacacaagctcaataagagacatgctaaatggatggaatttcttgaacaatttccttatgtcatcaaatacaagaaagggagcaccaatatagtggccgatgctctttctagacggcacactctcttttcaaaactaggtgcccaaattcttggatttgaccacataagagagctttatcaagaagatcaagaactctcatccatctatgcccaatgtctacatagagcacaaggaggttactatgtgtccgagggatatctttttaaagaaggaaaactttgcattccccaaggaacacatagaaaactccttgtcaaagaatcacatgaagggggactcatgggccattttggagttgataaaactctagacttttaaaagcaaaattttgttggccacacatgaggaaagatgtccacgacattgtctagagtatctcatgtttaaaagcaaagtctagaacaatgccgctggactctacacccctttgccgattgcaaagctccttgtgaagacattagcatggatttcattttaggacttcctaggactgcaagaggccatgactctatctttgtggtagtggaccgttttagcaaaatgtctcactttattccatgccacaaagtagatgatgctcaaaatatttctaaactcttctttagagaagtggtgagactccatggtctccctagaagtatagtgtccgatagagatcaccttaaatatataattatacacttgttttttttctcttttttattttatcaagtaaaaagtatttgttctagattattatgagtatatacttactttctgtatttcatttctttctattttttatgacgatgaaatttcttattatatccagacttttcatatatatttttatttcttttccatctagatgctctgtacttttcttcagttgaaatttccactctccaacaaaacatcattcaagttttgtataacactgtgacgttaaccagttaaaataagaaaatcatgtaatataaattatttcagtagatattttagaattacaaatacgataaataattaaatttaaaaaattattaaacaatgaatttttttggaaattaatataaaacttagacttgtggtttcttcattcagtcaaaacctttttctattgtgtggcgtgtgcgtgaacatcgaatttgggtgctttatgccgctttatcttcatctgcaccttcaaattaataatttaattccggaaaataataaacccacacactgttttatgcatatattaagataaataaaagagaactattttaaagaatataaaataataaatgtaacaaatgatgtcactaaagaagaaaaaaattaacaagaattgtaatatatttctttatgaaatgttttgtgcattaccgagagaggtcgaacatgatacacgcaagcatctaactagtttggtaattccttttcaacatcgntaagcacatcacactaaaattactttaaatagataaattagattcaattggatgacattaatttataatactctatccaaaattataactataaataaaaagttatttttagaaaataagtaatgaaaatttaattctaaaatttataacacttttatgctgtgtttgtttcgaagcatagaaaaataaaaagttattgttgggaatgaaaagtgaagaaaatcatgtaataaaaacaaaatgacacgacaatcaaaaaaaaagttttcatgcaaaacttttttcaaaatttacacttttatgatgtgtttgtttcgaagtgtagaaaaacgaaaagttattattggtaatgaaaagcgaagaaaatcacgtaataaaaacaaagcaagatggcacgacaatcaaaaaaaagtttctacacaaaactttattcaaaatttacaacacttttatgttgttgtttgtttccgaggtatagaaaaacaaagaattagtgttggtaatgaaaagtgaagaaaaccatgtaatgaaaacaaaatggcacgacaatcaaaaaaagttttcacgcaaaattttcttcaaaatttataacattttcatgttgtgtttgtttcaaagcctagaaaaacgaagagttactattggtaatgaaaagcgaagaaaaccacataataaaaacaaaatggcacgacaatcaagaaaaagttttcacacaaaacttttttcaaaatttactatgtttatttcgaaatttagaaaaacgaagagttattattagtaatgaaaagcgaagaaaactacgtaataaaaaacaaaatggcacgacaataaaaaaagttttcacgcaaaattttcttggtgcgcagaaagttatatatattaattaattaattttcatttacttttttccctttttattttaaagttaaattattattattttcatttaaaatataaatattatttaaatataaaaaatataaccttaatcaaaacaaagccttaatctaaaatttacaacacttttaaccttaaaattaactttaaaaggaaaatgatagtgtgacaactaaaaaagttgtatacaaccctgtcataggtttagaaataaatatatataataaagagtaaatttgtaattaaatgatataaaaaagtattaaaataataatatttagagtagtaatatggttgtataaaaaaatgtggttgtccatatatcattattcactttaaaatatcatgacaaatattttcaccgaaagatggaaagaacgaaaagagcgttggataatggaaaaatacaagcaatctccctccagtactttgcataacattttgtattagtgatgagttttttatcatatatatttagaatataggaaaattttagaatcacgtggatagctatataatagtaatattttaatttataatgtagttgattttatttgtcaactggtatacataaatatgtgttgatagtgggtgacttgtggcttaaagaaatgtccagaggctgacaacaactctgcacagactagcgtaaac

SEQ ID NO: 2 - DNA, 4921 bp, Phaseolus coccineus
  Scarlet Runner Bean G654 genomic region
gcatgcactgccacaagtagtgaactcatggttttacctcctcaagtagaaaaccttttgagtgaatttgaagatttattctcccaagaaggacccattgggcttcctcctcttagggggatagaacatcaaattgactttataccgggggcaagcctaccaaataggcctccttatagaaccaaccccgaggaaacaaaggagatagaatcacaagttcaagacttgttggagaagggttgggttcaaaagagcctaagcccttgtgctgtacctgtcttgttggtgccaaaaaaagatggaaaatggcgtatgtgttgtgattgtagagcaatcaacaacatcaccatcaagtataggcatccaatcccaaggcttgacgatatgcttgatgaattgcatgggtcaactctattctccaaaattgaccttaaaagtggatatcaccaaattcgaatcaaggagggtgatgagtggaaaaccgcttttaagaccaaatttggattatatgagtggttggtgatgccctttggtcttactaacgctccaagtacattcatgaggcttatgaatcacaccttgagggattgtataggtaaatatgtagtagtttattttgatgatatcttagtatatagtaaaaccctagaagaccatctaagtcaccttagggaagttcttctagttcttaggaaaaatagtctttttgccaatagggataagtgtaccttttgtgtagatagcgtagtctttttaggctttatagtaaaccaaaagggggtgcatgtagatcccgagaaaatcaaagccatccgcgagtggccaactccacaaaatgtaagtgatgtgagaagttttcatgggttagctagcttctatagaaggtttgttcccaatttttctagcctagcttctcccttgaatgaacttgtaaaaaaagatgttgcattttgttggaatgaaaagcatgagcaagcctttcaaaggctaaaagctcactcaccaatgcacccatcctatctcttccaaatttttccaaacttttggagatagagtgtgatgcatcgggagtaggcatagtgcggttttgttgcaaggtggacaccccttgcttattttagtgaaaaactccatggtgccaccctcactaccccacctatgacaaagactctatgctcttgtgcgaccctaaagacttggggaacactaccttgngtcccaaagaatttggntatccatagtgatcacgagtctttaaaatatttaaagggccaacacaagctcaataagagacatgctaaatggatggaatttcttgaacaatttccttatgtcatcaaatacaagaaagggagcaccaatatagtggccgatgctctttctagacggcacactctcttttcaaaactaggtgcccaaattcttggatttgaccacataagagagctttatcaagaagatcaagaactctcatccatctatgcccaatgtctacatagagcacaaggaggttactatgtgtccgagggatatctttttaaagaaggaaaactttgcattccccaaggaacacatagaaaactccttgtcaaagaatcacatgaagggggactcatgggccattttggagttgataaaactctagacttttaaaagcaaaattttgttggccacacatgaggaaagatgtccacgacattgtctagagtatctcatgtttaaaagcaaagtctagaacaatgccgctggactctacacccctttgccgattgcaaagctccttgtgaagacattagcatggatttcattttaggacttcctaggactgcaagaggccatgactctatctttgtggtagtggaccgttttagcaaaatgtctcactttattccatgccacaaagtagatgatgctcaaaatatttctaaactcttctttagagaagtggtgagactccatggtctccctagaagtatagtgtccgatagagatcaccttaaatatataattatacacttgttttttttctcttttttattttatcaagtaaaaagtatttgttctagattattatgagtatatacttactttctgtatttcatttctttctattttttatgacgatgaaatttcttattatatccagacttttcatatatatttttatttcttttccatctagatgctctgtacttttcttcagttgaaatttccactctccaacaaaacatcattcaagttttgtataacactgtgacgttaaccagttaaaataagaaaatcatgtaatataaattatttcagtagatattttagaattacaaatacgataaataattaaatttaaaaaattattaaacaatgaatttttttggaaattaatataaaacttagacttgtggtttcttcattcagtcaaaacctttttctattgtgtggcgtgtgcgtgaacatcgaatttgggtgctttatgccgctttatcttcatctgcaccttcaaattaataatttaattccggaaaataataaacccacacactgttttatgcatatattaagataaataaaagagaactattttaaagaatataaaataataaatgtaacaaatgatgtcactaaagaagaaaaaaattaacaagaattgtaatatatttctttatgaaatgttttgtgcattaccgagagaggtcgaacatgatacacgcaagcatctaactagtttggtaattccttttcaacatcgntaagcacatcacactaaaattactttaaatagataaattagattcaattggatgacattaatttataatactctatccaaaattataactataaataaaaagttatttttagaaaataagtaatgaaaatttaattctaaaatttataacacttttatgctgtgtttgtttcgaagcatagaaaaataaaaagttattgttgggaatgaaaagtgaagaaaatcatgtaataaaaacaaaatgacacgacaatcaaaaaaaaagttttcatgcaaaacttttttcaaaatttacacttttatgatgtgtttgtttcgaagtgtagaaaaacgaaaagttattattggtaatgaaaagcgaagaaaatcacgtaataaaaacaaagcaagatggcacgacaatcaaaaaaaagtttctacacaaaactttattcaaaatttacaacacttttatgttgttgtttgtttccgaggtatagaaaaacaaagaattagtgttggtaatgaaaagtgaagaaaaccatgtaatgaaaacaaaatggcacgacaatcaaaaaaagttttcacgcaaaattttcttcaaaatttataacattttcatgttgtgtttgtttcaaagcctagaaaaacgaagagttactattggtaatgaaaagcgaagaaaaccacataataaaaacaaaatggcacgacaatcaagaaaaagttttcacacaaaacttttttcaaaatttactatgtttatttcgaaatttagaaaaacgaagagttattattagtaatgaaaagcgaagaaaactacgtaataaaaaacaaaatggcacgacaataaaaaaagttttcacgcaaaattttcttggtgcgcagaaagttatatatattaattaattaattttcatttacttttttccctttttattttaaagttaaattattattattttcatttaaaatataaatattatttaaatataaaaaatataaccttaatcaaaacaaagccttaatctaaaatttacaacacttttaaccttaaaattaactttaaaaggaaaatgatagtgtgacaactaaaaaagttgtatacaaccctgtcataggtttagaaataaatatatataataaagagtaaatttgtaattaaatgatataaaaaagtattaaaataataatatttagagtagtaatatggttgtataaaaaaatgtggttgtccatatatcattattcactttaaaatatcatgacaaatattttcaccgaaagatggaaagaacgaaaagagcgttggataatggaaaaatacaagcaatctccctccagtactttgcataacattttgtattagtgatgagttttttatcatatatatttagaatataggaaaattttagaatcacgtggatagctatataatagtaatattttaatttataatgtagttgattttatttgtcaactggtatacataaatatgtgttgatagtgggtgacttgtggcttaaagaaatgtccagaggctgacaacaactctgcacagactagcgtaaacatgaagtccaattttgctattttcgtagtcttttctcttcttcttctggtacctcttcaatcttctctacaaaaactctgttgctctttcacctctgtttgtaattttgtttacacttttggaaaattgaagctgatatatatgtaacaacctttcagttttgtctgcactgaaactgatagaaaaatatacgttttgtggatatatataggttggcagttgcagctgcgcaagaaaagacatgagagggtattggaaggatatgatgaaggagcaacctatgccagaagcaatcaaagaccttattgaggattcagaagaagtgtcagaagcagggaagggtcgttttgttagggacttcgatgtaaagcctaatgtcatattatatcacacacatgttgtgcccatgaagcagaggcagaagaataaagattgaagactatgtgattggcagtttcagacttatttggcaccaaatttatgatgctcttgttgctgtttcaaaatttgtactcaaactttgaaccctttgcagcatcttgcttctttttggtcttgctgaattttgtcacagttatactgtcacgaatagtttctcttcataataagcaacttttcctctc

SEQ ID NO: 3 - protein, 90 aa, Phaseolus coccineus
  Scarlet Runner Bean G654
MKSNFAIFVVFSLLLLVGSCSCARKDMRGYWKDMMKEQPMPEAIKDLIEDSEEVSEAGKGRFVRDFDVKPNVILYHTHVVPMKQRQKNKD

SEQ ID NO: 4 - DNA, 6250 bp, Arabidopsis thaliana
  Arabidopsis G654 genomic region
caaaacaaaagcaaatgccggttttcttattattatttcgaactttagacctttttgtaacgtttctttaatttttttccttgataaagaaccctattatatcttagctaaatatttacctcattttgtttatgagctaaaccaccccaaaaatattgtagttttgctttcggatttaactgccaagcaagtgattagatatattaaaggaaaatgaatgaaaggacaaaaaaatataaacgacaatatttgaatactgatatttatctccattctcaaatatttttgatttattgtgacaatatttggttgtttcccatttgctacatctttgaggacatgaaatgataacatatatatgaacgagtataatacattctcgtttcattttacaaataatgtcaatttatgctaacattttttatttaaaaattatccttataagatttcagtgtattattttaccatggtactgtaaagtcggatgctatatatatatatatatatatatatatcaaaaatgacactgaagaatttatttgaactaaaactaaaaacgtaaaataaaaagaatttttcaaaaatcaaaaattttatataaaaatatagataaaatgttaatatagtacaacttctattcaaacagagagaataaatcttctatagacagtgaatatccattataataacgagcaatagttgtaatgttgcagtacaaaaagagaattgtaatatttgtgcatgattgagaaatctaagttgactttgaattaaaaggctaattccaacaagtacatgtagaagttgactatagctatatatttactacaaattgatcatttcaagaaagacatttaaattaagatatgcatgcatgacttgattgaaccccactcgcttgcttcgtgccattcgacaagatgttacttttaaatgcaaggtaaattatggatatactcttctgtattttttgtagtagatatttttacgaaaattgttttttttccaaaatcaaatgatatttattaattttcaatatagaattaattaaattttaattaattttgaagatttatatgctgcagattagattaccattggtgaaatcatgtttaggtaaataataaatgatgttgtagtttaggaaaaaaaaaaattctttaatctttatgtaagaatgttaaacttcaattataaaaatatgaagcagtattatataagatgtttaactaatcgaataatattttttgggatgaaattttcttgcatatgtttctaaaaaaataatatgtgaaaaattaacattcattgtatgtttataagaaatatatgtgagttttgtttagataaataatacttaaaattaagaatttgtaaagttatactgcacttcaaatatgttattttttccttttatttaaaatatcagcaacattctaaatgattttattttctttaaaaaattgaaaaaatgaaattagcaaatatgtaaaatttaaaacgaatttaagaaaaaactttgtaaagatatgatatgctttataaaaaaaacttggtggcgtacctactaaatatgatcacattagagatttgtatcctttagcatatagtatgtagtatagatatctatatttttatttattaaagagcatattcataatataggtattatatgttaattacaataaacgttcaattcgttatgttagtttttagaaaacttattgcgtgtgcatatcaatgtgagaaagcgactccacatgtgagatgttggtctgagaaagctttctgcacttggtcggaactacttcatggactagaatgcaatccatctattcaaagaaaagcagttgtccatgcatgcctcggtttttcacatttggaagcagcgcaacaatgtcttacataatatgcgatcgatcactctgcaaccaatattcaagtacatagaccatgacatcaaaaacattatcacaccgagaagaaagaaacgtcaatttggtaacttaatggcgttatgcctgcggtgaattctcctaagagttctcccaaattttattgattccttgtttttaactttttcgccaaagaatcatacatatagatttgacaccatttcaacttatcaaatacaagtgaataaataatttcaagcttgaaaggaatttaatcatgatctaaacctaaacgacaaattcttcacaagtgagaatcactaattgactaccccttggtcgcatatacatcattgttgtaaatctgaaaattggtttggatttgatctgatatgtcattcatataaaacttgtattatttattttagaattttgccgcaaacagataaatcatcatctatttagaaaattttcatttgcaccacaattaatcaggggaaaaggtgaaatcacatatcttatctacactctttattaattaaacgccataatataacaaattttcaaataccacttatgagaagcactaagatcacctttttctttatgactttctttctaaagctaagctggtagtcatgactcatgattatccttttcctaatgggaatattgtggaagcggtttcaaatctttagacaaaattccatggccactaaaagttagcaaagttaaaataagtttaaaaaaatatgagtgtacttggccatatgccatattgttgagatcataacaagagaaataatagtttattgaagtttagatcataatcacaatacatcattgccttcatcaacattttccatggatttgagaggatcaacttcaatactaatggtggggtcttattcatccattgctctctagccaattaagcagttaggttatttgtgtactctagtagttgccaaatcaatcttaatattcacaatgttgtaatttctaattacgtatagataaatgactagataacacgtggctttggttttatcaggaaagttttccaaatcatatatatgaatgtagaatagtgttcttcattaattattaattagcatctcaccatctgagactgggagcatgtgacaagttgacatgtgtattaagagaactttgagaaaaccacttttatgatactcccatctgagactgggatgagtaccattttataaaaatatgagtagtgaaaaaatattcaaaaaaaattctaacatgtcctttaaaacattttaaccttataattttaacaaacatcttccaatatgcgttatgaaaactttataaaacttttttataacatgcttttgaaaattttataaatctgtatttttagaaacaaagtgatacttttgaaaatagacaaatgaagtgctattttttaaaattgatatcataagtcttaactgtggtttgtttgaattttatttatatacttgtcaaaataaaactaaataaataaattaaattattttataatcatgaagataatattatcataaaagataaatataaaatcaacaaatttatatttgttaataaaaatactttgagctcttcttcataagacttttccagcttccatctagaaaatcacataaattaaaagataaataaccgaataaacatagttcacattctaactcttagtcttagatttgttttaattttcaaaggtttaggtattgtatatgttttttttattgggttgctagattttgatccaagaagaaatgacgggttgtagtatagatggtttgtttgagttttttccccttggtttacttcgtttggtttttgtccccagaattgttcttgtactcgctggtttatgtctctacaaagtccacgaccattgccggctctttgtatttcaacttgaattctaaattcgattgatgaaaaaaaaatgtatctcttaaagtccattagtaccaaaaataactatatcattactacataaaatagtcttgggttttccaaagtatttcgttgatatatgttaagagttcgaaatagacacatagatataatgttgaaatgggacctctcacataattatctccttttctcttcatttctctacctctcaagtttccaatcccaccctaaggtaatttatttcttaacctaagtaaatttgttaacaaatcttaactagctacaaatgtgtattacaagtcttaaataaaaacctactttaattcaaaggtattaaaccttcctaaattgatacttacttagtatcgatcggtctagtttagggtttggacaacacaccatcatggggacgaaattagtcattctacggtgtccaagacacaaatctcggactcgatgtggatatgacacttcattataacttttaacttcataaaaactaactattaggaggaagaatcggaatctgcatatcaatcacaatagactatagtatacttagattttgatctaatcaatgggctccttcaactaataagtagcccactaccaataatgaaatcataagacattattaaattaatcaatgttctaaaaatactttggttatgtgtcccgtagagctaatgtgcacacacaatgaaagttgacccgtttcacttgtcccacttttatgatcttttcttttaggttaaatccaacttttataatctcatcttgttatcaaacaaaacttttggcctgtctttttcataatttaaagtaactctcacggagaaaagccaacattttcttcttgttttattctttttaagaaaaatgaattcaaggggaccccaaatttaaaaggaaaaccaaaactcctttctatgtatttattacttgaagttttctatgtaatcaacaatcctaacagtagagaataaaaaacatcgttttgggaggttttatattagcatatgagaatagttctaaaattgttttacacaaaaattagattttcttttcctctgtcaatggagctatatcacttgtcattttgcttaaccctttgcgggaagattgttatgaaacagttttaatggaattctagttgccaatgtcacgtttaatatgttttgtccctatactttattgaatcttataatctttgttatagaattatctacttttagtattttacattaacataatctatagaattcttctttgttctatacaattaaacaagtaatatattcttaatacatattaaaaatggtggtgttgctatctgagctgtaatagttgattgctccagagaagaatagacaaaaatccttacttaagaggcccaccactctgaaaatttagacaagaaaaattaaacaaaattaggttacacatattatcatttatatatatgcacaacacaaagttgaccttgcaatgtactattgaataaaataaataaatgcaagaagagagggaattatcactgttaccaagaaaacaacttcctctaaacaggtctctatatatataaactttaacacctaaagaattaacacagatcaagaaaaaatcctcaaaacaaaagttaaagcagacatgaagcaacagcaacgttacttggtcgtcttcatcgtccttttaagctttcttctggtaaagcttcttccttaattatattaaaaccctaattaagatctcatatatctgaatgttgtatatatttgttggtatagtttgtgaatctgagtgaaggaagaacaggaggagttgcagaagaatattggaagaagatgatgaagaatgaaccgttgcctgaaccaatcaaagagcttctcaacaatccttttaggaccgcacaagagagattcatccagaatttcgacaccaaatctgttgtcatcatctaccacaatcctaatgaataatcaatgaagtctctcatatagatatctatgactttaatttgtgtttatgtatggatcgacttatacgtgcacgtatatgttattaattaagaaaagaaaaagctgcttgagttgttgtgttatacacgtatactaaatatgttctgtttagtgcagaaatgttaaccctagctataagggattttttgttcttttttttttgttaccattaatgtgagtgagtgagttttgtgtgatgaaaattagatttgcttcacattttgttttgatatatataaatcaatatactgtgcctttcgtgtcttgtttcttatattattttgtgacattaattaattatcttatcaaaaatttattttattaactgtgtcctatggaaaaagatgaacaatatgagttaacctcatctcaaggagattcttttttgttttgtttttc

SEQ ID NO: 5 - protein, 82 aa, Arabidopsis thaliana
  Arabidopsis G654
MKQQQRYLVVFIVLLSFLLFVNLSEGRTGGVAEEYWKKMMKNEPLPEPIKELLNNPFRTAQERFIQNFDTKSVVIIYHNPNE

SEQ ID NO: 6 - DNA, 4846 bp, Phaseolus coccineus
  Scarlet Runner Bean C541 genomic region
aagctttacaaatgtcccccaaagatgaaaccacgttattattagtaaatcctgaaaaggttaacgcttctgttcctcgaattctaaaccatctgaaatatctagtggtttaaaatggagacttgaggatatagtctcctgaaccagctgtcacggctgagttagataacattactgaatttctacgggagcggttgaaatcactttcgcccctttaagaagaagcctacaccgggcaccttctttacgcaattcgaaatttagtcttgccaggcagtcgttggatcgaaggtctttttcgataccgaggaatctgactttgcaaggaataattcctaatcacaccaccccaacccctgaatacacttcaggaccctctgaaaccaacttcgtttcggctaaatcacaagaatctcccactcattccgattttagccaattaaatatgatatcggtctgggaagccgataaggaaattctacaaaaagagtttatgaatgaggaaaataaggaaaagagagaactattttttaggtaccctgaaagagaacgagaaaaatttagaaaaaaatactactctcatctgtacactgttcaaaagaatatccnnnnnaatggttagataatataagaaaaggataagtatgattaaactgaaaccacgtcggcagaaacaaagtgaattcccccctttagaggaagttcgtttcttaaatatagaaaacaaagaagtagtcgcctccccttttaaaatgatctcagaaaaacgagaagtaagtataaaagatattcaaaatctacacagtcaactaaattttactaatcaaatgctttttcaattagcaaataaaaaacaaaagaaaaaagmgaaaattgaagaaaaatcgttaataaaaccatttaaattctcagaagaagagataaaacagttaaaaattggtcaaactttggattctttatacgatgaagtaaaacaaaagttatctatctcggtaataaaagaaaaaccgaaatctaataatgatatgcccaaaaggacaaatccaaatcaagaagttttagacgaaatcgaaaagagattaaaacaaactctgaacgacacaataaatgtgatagaagaaactaaaaactcagactcatgttcagagtctcccgatcgtattgaaaaaataaaacgtaataaatcagagatttccagtaagccgaaatttttacactcgccccaccttcgatatcatcgagatggcgatggacacctcagcattgatggaatggatactgagtgatatgatggatgacagatgatgaatatagaaaaactcacgaaataacaatggccgctacagcatatagagtaaaacataccgaggaacaaacaataaaattaattatatctggattcacgggagtattaaaaggctggtgggataattacctcatgccagaacaaaagaattatgttctaagctgtgtaaaaatagaaaacgaagaaggaataccactaatggtggaaacattggtggtagcaataattcataactttataggagatccaaagatttttgaagaaagaacatctttattacttcataatctaagatgtccaaccttaggtgactttagatggtattcagaaaattttttagctatggttttaacaagggaagattgtagagaacctttctggaaagaacggtttatagctggattaccggatatctttgctgaaaaggtaaaagaaaatttacaaaaggaatgcccaaacacccaattaaaagatgtaccatacgggaaaataagttcagttgtaaaaaatacaggtcttcagttatgcaataatatgaaaatagaaaataagataaaaaagagtgagagtcagggcatcaaggaattaggggaattttgtactcaatacggttatgaacgaaatacccctccatcaaaaaataaaaagaaaatagcaaaaagaagaacagggagaaacaagcgctaaaacaagcgctaaaccagcacgtaaaaattttagaaaaacggttaattttagaaaaccatgaaagtctaatgataagcccactatagtctgttataaatgtggacgcataggacacatgaagcgagactgtagactaaaagaaaaaattagtaatttgaccataagtgatgaattaaaagaacaaatggaaaaacttctgataaattcctccagaagaggaagaaacagaagaatcaataggagattctgattacgaagtattggacatgaggataacaattgtaattgtgtctataaaataaatacgataagtagtgaattaaaatttgcgttagattgcattgataaaattaataatccggaggaaaagaccaaagccttaatagacatgaaaaggctactcgttgaaaaagatgaacccagttcatcttcacaaaaacctgaatttataggatatgattttaaagaaatattgagaaaagcgaaaacatcacataaagaaataaccattagcgatcttaatagtgaaataaataaattaaaagccgaaatcgaatctataaaagtcgagctacaagaattaaaagataaaattatacatgaggaatccatctcctctgccgacgaaaattcacaagaagaggaagctagtagaccttccatcaaagaaataacatacaaaagacaaaagtggcatgtaaaaatagccctagaatttgtttgttttgtgaccgtttcattgtggtcaaagatgagtccttacctaacacaataaaaaacgttactcttaaatatcaaaggagagctacaaatatcaatgaatgaatgacattaatatttttctttagttttaaaacttgaatgagttgttttcataaatatctgactgactgacatttttattttttctgaaaatgaggaaggtttattacgttaacaccatatatatatttttatctcaaagtcaacgaaatattataaaagaatcaattaaaaaaaattattcttttgcagaaaaaaaaattaaaaatatgaaactcctccacaccatattaccatattataaatataaaaaaacctctcacaaatgtgcattctggaattctttatgttgagagattaatctctaaagaaaaaaggttgagaaaggtgcagcaacaatgtctccattctgtagaaacttttcaatggcatgggtgcttatggcatttgtgttgtttgcaaacagtgctatgcccacaaatggatccactgttggggtaaaaaacatgttgggtggtaaattgatgctaaacgttttatgtccccatattgataagcaacacattatcccgaatggtggttcatttgagtggaagtacaatggtggtgctccaccaataggacaatcaccattcatgtgtttctttcggtggaataatgttcatcactcccttgatctgtgttcaccaagcaagtatactggttgtgaaaatgccatttgggaaatcaaagaaaagcaattttgtaggtacagaggtggacctattaattatttttgctatgactgggatgattagttatatagattattcatgtttcatctcaataaaaaaatgactttagagtgattcttagtttgcttaacattcttacatattcctaactattccgtcactaccacccgtaactatatttatttaaaattagtatctgtcacagttttatttttaaaaaaggttatgtggattagaagagagataaatatgtagacggtcaccaaccttaatttttgaactatgtaagactatattgaccaagaatatatgtttaaactcattcatttaaagactatatctccatttatgattatgcaaatgcaattagttttttttttcattgaagaattcaaaagaaagttatcattaaaaagtatcattaaatcacttatatgttgtttcttaatatccttattgttaatagaataattttttttatcctttaattaaggttattactacttttttttcatatcttcattattttgaaatatttttaaaatttatcaatttttgtaacaccccagaaaatacatgtaactatcactttttttttatattacaaatttatgacttatagaaatacaaatattaaaaatataaggttcaaaactacatcctaaagtctttcagaccctctgacacatgtatcatctgctcgtatatgtgatacagtcatcgcagttcacaagataacaagaaaaccaagggtaagctaatgaaaaaaaattccataacatatttaattcatgcaaaaagaaccagtcaaagtaatcatttataaacatttctttaaatattgttatataaaatttcaatatcaatttcatcattcatatagaccacacatggatctattttcaatcacaatcattggatttcattttaatcctacttcgncttccagaagactcattaagtatgcccctaccagagactaacacctaatcaaagagaaatgatcaaggtaagttcaaacatccaataacgagtgcctacagtgggacccaatgtgtatgaactccttatcagcttctcaccacctgatatcttattctatatgacgtagatcatcagtgaaactagaggatctccgttaaacatatgttttttatacttaatgtcatcaaacaacaactcacacattatcccaaatgtatgacatcaatttcatacaattttcatcattcatatataatacatatcattgaatcacataacatttaaaaattcataccattcaagaacttttccaacatcaaaagcaatatttactttcaaactatcaaaatataattattatttaataaagctt

SEQ ID NO: 7 - protein, 132 aa, Phaseolus coccineus
  Scarlet Runner Bean C541
MSPFCRNFSMAWVLMAFVLFANSAMPTNGSTVGVKNMLGGKLMLNVLCPHIDKQHIIPNGGSFEWKYNGGAPPIGQSPFMCFFRWNNVHHSLDLCSPSKYTGCENAIWEIKEKQFCRYRGGPINYFCYDWDD

SEQ ID NO: 8 - DNA, 2601 bp, Arabidopsis thaliana
  Arabidopsis C541 genomic region
ttatcttatttccatataattgttgttttactttcaaaatttttaattttttatatttatctttttacagtttaaaattaataaaatgaaactttttttcttaaatgtgttaaaatataaaatcaaaaaagttgttatatggtacatggcacaatcttataaattattaatttgaaaacgatactttatataataaaattatcttagttgacatttttattagtgttttcaatcatatttttgtttgcttgataagcgtaaaacaaatcaaacttaacgatactttatataataaaattatcttagttgacatttttattagtgtcttcaatcatatctttgtttgcttgataagcgtaaaacaaatcaagtaaagttgggcacctcaattgttttaaaaaagtttgggtacctcaaaaattaataggtcttgtcagattcttacaaaaaaaatctggaagaatttatgaaagaaggggggggagggggggaggggggggaagtgaagatgaatattcaacaaaagagggtaggcatgatgttaagtgagttaaaaaactatgttaatggagacaattttctgttaacaaacccgttaattgaaaacgatagcattcttctctaacaatgtaaaacgatattgttttatcataactactcattaaatttctgagtttcaaatcatataaagatttaggggggtgtattcaattaaggatttgaaatgatttgtattaaaatgacaaatcccatgttatttcaaacatgaattgtaaaaacttttttaaaatcaagtgttattagattagtgattttaaaatgtacaaccaaacccactgttattggaaacattttaagtagtggatttaaaatgacttgagtgattttgggtgggattgcagaaaatttcttagttaagaattcaaacatccaaatctcatggtttcaagtagaatttgggagaattttaataacaaatctcctaatttaccaaaagtcaccaaaatcatttaaaaactcattaaaatttaaatgatttcaaatctccagttgaatacatccccttggaattagagattttgctcgatttgggacctaagattgaattttggggatttagtttaatcgttacaacaaaatgacatcgtattattgttataggaaacaatgtcgttttcagttgacatgtatgttaatagaaaattaactctattaacgggatttgctaacccatttaacatcgtaactaaatggtcaagtcaataaaagtttggtatttatttgaaaagtcaacgtaagtttgatatttatttgaaaagtcaacataaatttgatatcttatttcgtttcgacagacataaggatttacatcaatgtttttaataaattaaagattattatgacattttttccatttaaaattgccaatgttttcgaaaccaagatactcaaaattgacatacctaattcaatctacatttgtttgacagcaattcacgtgccttgaccacatggcacatactggcaatacatcaattttaaggaaaaggtagattcggatacaatataatggaaataagtggaaaggatcattgactacttgacttgtaacaaacaacacacagtatataactcattcgacatttacaaacaacattgtgctagcttaaactccctctcctattcaaaaaaatggatattccaaagcaatatctatcactattcatattgattatcttcataactacaaaattatcacaagccgaccataaaaacgacattccagttcccaacgatccatcatcaacaaattctgtgtttcctacctcgaaaagaaccgtggaaatcaataatgatctcggtaatcagctaacgttactgtatcattgtaaatcaaaagacgatgatttaggtaaccggactctgcaaccaggtgagtcgtggtcttttagtttcgggcgtcaattctttggaaggacgttgtatttttgtagttttagttggccaaatgaatcgcattcgttcgatatatataaagaccatcgagatagcggcggtgataacaagtgcgagagcgacaggtgtgtgtggaagataagaagaaacggaccttgtaggtttaacgatgaaacgaagcagtttgatctttgttatccttggaataaatctttgtattgacaacaatatgctgatgttctgtcttttacgactcatggagtttcattgtttgaaacaataatataaaacatataaaatttctattattccaagttccaacttataataatttgataatcatatcatattatcatcttaagcattcaatgctacaaagataatacccccaagctattttacattaaaagctgaaacagagacacaatactaacgataaaagttcgtagtatctttatgcaaccatacatacatatacacaaagatagacaggtagtgtcctaataattctacttgggtgaggtatgaacagcagcaacagtagataccattgtatccataccacacatattatgaggccctctgcagattttgtagtaaccatgctctccccacatcgctccccacgagttcttgataatccaa

SEQ ID NO: 9 - protein, 161 aa, Arabidopsis thaliana
  Arabidopsis C541
MDIPKQYLSLFILIIFITTKLSQADHKNDIPVPNDPSSTNSVFPTSKRTVEINNDLGNQLTLLYHCKSKDDDLGNRTLQPGESWSFSFGRQFFGRTLYFCSFSWPNESHSFDIYKDHRDSGGDNKCESDRCVWKIRRNGPCRFNDETKQFDLCYPWNKSLY

SEQ ID NO: 10 - DNA, 10 bp, Artificial Sequence
  Description of Artificial Sequencepromoter control region of Scarlet Runner Bean G564 and C541 promoter region
gaaaagcgaa

SEQ ID NO: 11 - DNA, 10 bp, Artificial Sequence
  Description of Artificial Sequencepromoter control element of Scarlet Runner Bean G564 promoter region
gaaaagtgaa

SEQ ID NO: 12 - DNA, 10 bp, Artificial Sequence
  Description of Artificial Sequencepromoter control element of Arabidopsis G564 ortholog promoter region
gaaaagccaa

SEQ ID NO: 13 - DNA, 450 bp, Phaseolus coccineus
  Scarlet Runner Bean G564 promoter (-921 to -662) PLACE database Signal Scan search sequence
tgaaaagtgaagaaaaccatgtaatgaaaacaaaatggcacgacaatcaaaaaaagttttcacgcaaaattttcttcaaaatttataacattttcatgttgtgtttgtttcaaagcctagaaaaacgaagagttactattggtaatgaaaagcgaagaaaaccacataataaaaacaaaatggcacgacaatcaagaaaaagttttcacacaaaacttttttcaaaatttactatgtttatttcgaaatttagaaaaacgaagagttattattagtaatgaaaagcgaagaaaactacgtaataaaaaacaaaatggcacgacaataaaaaaagttttcacgcaaaattttcttggtgcgcagaaagttatatatattaattaattaattttcatttacttttttccctttttattttaaagttaaattattattattttcatttaaaat

SEQ ID NO: 14 - DNA, 448 bp, Phaseolus coccineus
  Scarlet Runner Bean G564 promoter (-921 to -662) PlantCARE database Signal Scan search sequence
gaaaagtgaagaaaaccatgtaatgaaaacaaaatggcacgacaatcaaaaaaagttttcacgcaaaattttcttcaaaatttataacattttcatgttgtgtttgtttcaaagcctagaaaaacgaagagttactattggtaatgaaaagcgaagaaaaccacataataaaaacaaaatggcacgacaatcaagaaaaagttttcacacaaaacttttttcaaaatttactatgtttatttcgaaatttagaaaaacgaagagttattattagtaatgaaaagcgaagaaaactacgtaataaaaaacaaaatggcacgacaataaaaaaagttttcacgcaaaattttcttggtgcgcagaaagttatatatattaattaattaattttcatttacttttttccctttttattttaaagttaaattattattattttcatttaaaa

SEQ ID NO: 15 - DNA, 10 bp, Artificial Sequence
  Description of Artificial Sequencesite #S000067 MARTBOX promoter control element
ttwtwttwtt

SEQ ID NO: 16 - DNA, 10 bp, Artificial Sequence
  Description of Artificial Sequence3-AF1 binding site promoter control element
aagagttatt

SEQ ID NO: 17 - DNA, 10 bp, Artificial Sequence
  Description of Artificial SequenceHordeum vulgare ABRE and Petroselinum crispum ACE promoter control element
actacgtaat

SEQ ID NO: 18 - DNA, 12 bp, Artificial Sequence
  Description of Artificial SequenceSolanum tuberosum AT1-motif promoter control element
ttttattttaaa

SEQ ID NO: 19 - DNA, 10 bp, Artificial Sequence
  Description of Artificial SequenceTC-rich repeat promoter control element
gttttcttca

SEQ ID NO: 20 - DNA, 10 bp, Artificial Sequence
  Description of Artificial SequenceTC-rich repeat promoter control element
attttcttca

SEQ ID NO: 21 - DNA, 10 bp, Artificial Sequence
  Description of Artificial SequenceTC-rich repeat promoter control element
gttttcttcg

SEQ ID NO: 22 - DNA, 10 bp, Artificial Sequence
  Description of Artificial SequenceTC-rich repeat promoter control element
tttttcttga

SEQ ID NO: 23 - DNA, 10 bp, Artificial Sequence
  Description of Artificial SequenceTC-rich repeat promoter control element
tttttctaaa

SEQ ID NO: 24 - DNA, 10 bp, Artificial Sequence
  Description of Artificial SequenceTC-rich repeat promoter control element
attttcttgg

SEQ ID NO: 25 - DNA, 10 bp, Artificial Sequence
  Description of Artificial SequenceArabidopsis C541 promoter control element
gaaaattaac

SEQ ID NO: 26 - DNA, 10 bp, Artificial Sequence
  Description of Artificial SequenceArabidopsis G654 promoter control element
gaaaaccaaa

SEQ ID NO: 27 - DNA, 10 bp, Artificial Sequence
  Description of Artificial SequenceArabidopsis C541 promoter control element
gaaaatttct

SEQ ID NO: 28 - DNA, 10 bp, Artificial Sequence
  Description of Artificial SequenceArabidopsis C541 promoter control element
gatacaatat

SEQ ID NO: 29 - DNA, 50 bp, Artificial Sequence
  Description of Artificial Sequence oligonucleotide linker-primer
gagagagagagagagagagaactagtctcgagtttttttttttttttttt

SEQ ID NO: 30 - DNA, 16 bp, Artificial Sequence
  Description of Artificial Sequenceanchor/ reverse primer G primer
aagctttttttttttg

SEQ ID NO: 31 - DNA, 16 bp, Artificial Sequence
  Description of Artificial Sequenceanchor/ reverse primer C primer
aagctttttttttttc

SEQ ID NO: 32 - DNA, 13 bp, Artificial Sequence
  Description of Artificial SequenceH-AP49 forward primer
aagctttagtcca

SEQ ID NO: 33 - DNA, 13 bp, Artificial Sequence
  Description of Artificial SequenceH-AP50 forward primer
aagctttgagact

SEQ ID NO: 34 - DNA, 13 bp, Artificial Sequence
  Description of Artificial SequenceH-AP51 forward primer
aagcttcgaaatg

SEQ ID NO: 35 - DNA, 13 bp, Artificial Sequence
  Description of Artificial SequenceH-AP52 forward primer
aagcttgaccttt

SEQ ID NO: 36 - DNA, 13 bp, Artificial Sequence
  Description of Artificial SequenceH-AP53 forward primer
aagcttcctctat

SEQ ID NO: 37 - DNA, 13 bp, Artificial Sequence
  Description of Artificial SequenceH-AP54 forward primer
aagcttttgaggt

SEQ ID NO: 38 - DNA, 13 bp, Artificial Sequence
  Description of Artificial SequenceH-AP55 forward primer
aagcttacgttag

SEQ ID NO: 39 - DNA, 13 bp, Artificial Sequence
  Description of Artificial SequenceH-AP56 forward primer
aagcttatgaagg

SEQ ID NO: 40 - DNA, 20 bp, Artificial Sequence
  Description of Artificial Sequenceoligo(dT-20) primer
tttttttttttttttttttt

SEQ ID NO: 41 - DNA, 21 bp, Artificial Sequence
  Description of Artificial SequencedT-20dN
ttttttttttttttttttttn

SEQ ID NO: 42 - DNA, 34 bp, Artificial Sequence
  Description of Artificial Sequencemutagenic oligo
attggactgcatgcttacgctagtctgtgcagag